(19) United States
(12) Patent Application Publication
(10) Pub. No.: US 2010/0280268 A1
(43) Pub. Date: Nov. 4, 2010

(54) HOMOGENEOUS TIME RESOLVED FLUORESCENCE BASED TEST SYSTEM

(76) Inventors: Géry Karel Julia Dams, Paal-Beringen (BE); Inge Vereycken, Deurne (BE); Koenraad Lodewijk August Van Acker, Temse (BE); Emmanuel Marie Paul Ernest Gustin, Vosselaar (BE); Wim Gaston Verschueren, Berchem (BE); Asa Catrine Ohagen, Mechelen (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/293,589

(22) PCT Filed: Apr. 6, 2007
(86) PCT No.: PCT/EP07/53417

§ 371 Date: Sep. 19, 2008

(30) Foreign Application Priority Data
Apr. 6, 2006 (EP) 06112283.4

(51) Int. Cl. C07C 69/76; G01N 21/64; C07C 63/00; C07C 229/54

(52) U.S. Cl. 560/13; 436/172; 562/430; 560/44

(57) ABSTRACT

The present invention concerns a fluorescence resonance energy transfer based high throughput test system to measure the formation of the HIV gp41 six-helix bundle. In a first embodiment the current invention relates to a homogeneous time resolved fluorescence-based test system comprising a first helical polypeptide consisting essentially of the sequence of IQN36 (SEQ ID NO:1); a second helical polypeptide consisting essentially of the sequence of C34 (SEQ ID NO: 2) wherein said IQN36 is labeled with a light emitting fluorophore and said C34 is labeled with an ultra-violet excitable fluorophore.

### CROSS REFERENCE TO RELATED APPLICATIONS

This application claims priority of the benefits of the filing of Patent Application No. EP 06112283.4 filed Apr. 6, 2006, and PCT Application No. PCT/EP2007/053417. The complete disclosures of the aforementioned related applications are hereby incorporated herein by reference for all purposes.

Current therapy for the treatment of human immunodeficiency virus (HIV) generally targets the viral enzymes reverse transcriptase and protease. However, several other gene products of HIV, such as the envelope glycoprotein, also play critical roles in infection.

The envelope glycoprotein consists of two associated subunits, gp120 and gp41, generated by proteolytic cleavage of the precursor gp160 protein. It resides in the viral membrane as a complex of three gp120 and three gp41 subunits. It is the gp41 subunit that mediates fusion of the membranes of the virus and target cell, allowing the HIV to infect new cells. The gp120 subunit is involved in target cell recognition and receptor binding.

The process of membrane fusion mediated by gp41 involves a conformational change in the glycoprotein, exposing in the target cell membrane a trimeric coiled coil formed by alpha helices from the N-terminal region of each of the three gp41 subunits (the N-helix). This coiled coil interacts with alpha helices from the C-terminal region of the three-gp41 subunits (the C-helix). The resulting hexameric alpha helical interaction between the N-helix and the C-helix regions of gp41 fuses the viral and cellular membranes.

Proteolytic studies were used to identify the gp41 segments responsible for formation of the hexameric fusion intermediate, called N36 and C34. (D.C. Chan et al., Cell 89:263-273 (1997); incorporated herein by reference). Intriguingly, residues within the N36 and C34 regions are some of the most highly conserved residues of the envelope coding region of the HIV genome. Several mutations that inhibit membrane fusion and abolish infectivity map to these regions. Moreover, nanomolar concentrations of synthetic peptides corresponding to these regions have been shown to inhibit HIV infectivity and syncytia formation in cell culture, suggesting that they act as inhibitors of gp41-mediated membrane fusion. These results indicated that an isolated complex between peptides comprising amino acids sequences from the N36 and C34 regions would be a good model of the gp41 fusogenic intermediate.

Synthetic N36 and C34 peptides were shown to form a stable hexameric structure under appropriate conditions in vitro, indicating that peptides containing amino acid sequences from these regions can form the hexameric gp41 core in the absence of the remainder of the gp41 subunit. The X-ray crystal structure of the N36/C34 complex was determined by D. C. Chan et al., Cell 89:263-273 (1997). The structure revealed the three C34 peptides representing the C-helix of gp41 were packing in an antiparallel fashion against the three N36 peptides representing the N-helix, forming a six-helix bundle.

The N-helices form an interior trimeric coiled coil with three hydrophobic grooves. The hydrophobic cavities are filled by three residues of the C-helix, Trp 628, Trp 631, and Ile 635. In contrast, residues of the C-helix such as Met 629, Gln 630, and Arg 633 lie on the outside of the hexamer, and make no contacts with the N-helix.

The N-helix residues that form the hydrophobic pocket (residues 565-577) are highly conserved among HIV strains. The RNA that encodes these residues is also part of the Rev-response element, a highly structured RNA critical for the viral lifecycle.

The C34 peptide has been shown to be a potent inhibitor of HIV infectivity and membrane fusion. Alanine mutagenesis studies on C34 showed that mutation of the residues corresponding to Trp 628, Trp 631, or Ile 635 to alanine had significant effects on both C34 inhibition of membrane fusion and on complex formation with N36. Alanine mutation of either Met 629 or Arg 633, which do not contact the N-helix, had no significant effect on either membrane fusion or on C34/N36 complex formation. (D. C. Chan et al., Proc. Natl. Acad. Sci., U.S.A. 95:15613-7 (1998); incorporated herein by reference.) These data indicated that the hydrophobic interactions involving residues 565-577 of the N-helix and residues Trp 628, Trp 631, and Ile 635 of the C-helix play an important role in stabilizing the hexameric alpha helical bundle for membrane fusion.

This hydrophobic interaction is an attractive target for candidate gp41 inhibitors. While this interaction is present in the N36/C34 peptide complex, this complex is not ideally suited for screening of potential gp41 inhibitors because N36 is insoluble and subject to aggregation in the absence of C34. (M. Lu et al. Nat. Struct. Biol. 2: 1075-1082; D. M. Eckert et al. Cell 99: 104). Therefore, a soluble fusion peptide comprising the C-terminal 17 residues of N36 and 29 residues of GCN4-pI_{Q}I was constructed (with a 1 residue overlap between the two regions, making the peptide 45 residues long). (D. M. Eckert et al. Cell 99: 105; incorporated herein by reference). This peptide, called IQN17, has the sequence RMKQIEDKIEEIESKQKKIENEIARIKKLLQLTVWGIKQLQARIL (SEQ ID NO: 3), with the HIV-1 gp41 sequence of amino acids 565-581 underlined. IQN17 includes 3 mutations of surface residues in the GCN4-pI_{Q}I region to improve solubility. It is fully helical, with nearly the same superhelix parameters as the gp41 N-helix, and forms a stable trimer in solution. The X-ray crystal structure of IQN17 in complex with a cyclic peptide (D-peptide) showed that the hydrophobic pocket formed by residues 565-577 is nearly identical to that of N36. (WO 00/06599).

International application WO 00/06599 describes the use of IQN17 as a mimic of the N-helix region of gp41 for identification of candidate D-peptide membrane fusion inhibitors. A library of D-peptide molecules designed to present hydrophobic moieties into the binding pocket of IQN17 was tested in screening assays using mirror image phage display. However, to date, the use of IQN17 has been limited to D-peptides. Furthermore WO 00/06599 does not provide a simple assay for identifying anti-fusion compounds.

Inhibition of the formation of a stable six-helix bundle offers an interesting approach to prevent HIV infection. As mentioned above, HIV is surrounded by a lipid bi-layer bearing envelope proteins composed of three heavily glycosylated gp120 proteins on the exterior and three gp41 transmembrane glycoproteins on the interior. The molecular sequence of gp41 includes so-called “heptad-repeat” regions (HR1 and HR2 respectively). A heptad-repeat is a type of tandem repeat sequence in which a group of seven amino acids occurs many times in a protein sequence. Entry of HIV into the host cell begins with the binding of gp120 to the cellular CD4 receptor and its subsequent binding to the chemokine co-receptors CCR5 or CXCR4. This triggers a so-called spring-loaded mechanism that unmasks the gp41 fusion domain, causing it to spring toward the cell membrane. The HR1 regions then fold over into the hydrophobic grooves formed by the corresponding HR2 regions, resulting in a stable 6-helix bundle. This brings viral and cell membranes into proximity for fusion and entry. Hence, interfering with 6-helix bundle formation prevents the virus from entering the cell.

On another aspect, goals of drug design initially comprise the characterization of selectivity and affinity, efficacy, toxicity (therapeutic window/safety margin), pharmacokinetics, and stability, for a given compound. One of the earlier steps in drug design and discovery is focused on discriminating potential active compounds amongst a large variety of chemical compounds. This discrimination of potential active chemical structures is suitably accomplished by ligand-binding assays. Ligand-binding assays measure the affinity and/or degree of a drug to remain associated with a receptor. Within a molecule structure, the affinity or degree of binding of a specific moiety for a target recognition site of the receptor is particularly useful information in designing and optimizing effective compounds against a given target. Additionally, ligand-binding assays are especially convenient in elucidating mechanisms of action or inhibition.

Although screening assays for fusion inhibitors have been available for some time, mechanistic studies of HIV fusion inhibitors targeted to gp41 have been hampered by the lack of sensitive methodologies, which may discriminate between a large range of degrees of binding, leaving a tremendous range of chemical diversity untested.

One of the known screening assays is the so-called capillary zone electrophoresis (CZE) allowing the detection and discrimination of compounds or moieties of compounds useful for designing HIV fusion inhibitors. With CZE the degree of complex formation between two helical peptides can be identified. The endpoint of this technology relies on disruption of the complexing peptides. However, compounds inducing subtle distortions of peptide interactions exhibit equal anti-fusion activity.

A model and the means for directly measuring ligand-binding with high sensitivity and robustness is highly desired for screening a broader window of anti-fusion compounds.

In accordance with the present invention it has now been found that a fluorescence resonance energy transfer based test system detects distortion as well as disruption of the peptide interaction by any tested compound, because angstrom scale distance changes lead to signal changes. In addition the throughput, by using the test system according to the invention, for compound evaluation is much higher compared to e.g. the CZE technology.

The test system according to the invention also circumvents the classical problem encountered in fluorescence assays of intrinsic fluorescent properties of many test compounds by using europium. Europium exhibits an exceptionally long-lived fluorescence and thus the energy transfer to allophycocyanin is measured milliseconds after excitation when intrinsic compound fluorescence is less significant.

The present invention relates to a fluorescence resonance energy transfer based high throughput test system to measure the formation of the six-helix bundle.

Many compounds and proteins present in biological fluids or serum are naturally fluorescent, and the use of conventional fluorophores may lead to serious limitations of sensitivity. Most of these background signals being short-lived, the use of long-lived labels combined with detection on a time-resolved fluorescence basis surprisingly allow the minimization of prompt interferences.

In the fluorescence resonance energy transfer technology the dependence of the energy transfer rate is postulated on the inverse sixth power of the distance between an excited fluorescent donor and a nearby acceptor molecule.

In a first embodiment the current invention relates to a homogeneous time resolved fluorescence-based test system comprising a first helical polypeptide consisting essentially of a sequence derived from the heptad-repeat 1 (HR1) region of Human Immunodeficiency Virus (HIV) and a second helical polypeptide consisting essentially of a sequence derived from the heptad-repeat 2 (HR2) region of HIV.

The first helical polypeptide is labeled with a light emitting fluorophore while the second helical polypeptide is labeled with a ultra-violet excitable fluorophore. Alternatively the first helical polypeptide is labeled with a ultra-violet excitable fluorophore while the second helical polypeptide is labeled with a light emitting fluorophore.

In a preferred embodiment the first helical polypeptide consists essentially of the sequence of IQN36 (SEQ ID NO: 1) while the second helical polypeptide consists essentially of the sequence of C34 (SEQ ID NO: 2) wherein said IQN36 is labeled with a light emitting fluorophore and said C34 is labeled with an ultra-violet excitable fluorophore.

Alternatively the IQN36 sequence is labeled with a ultra-violet excitable fluorophore while the C34 sequence is labeled with a light emitting fluorophore.

The IQN36 sequence may comprise a linker between the label and the IQ-moiety of the IQN36 sequence. Preferably the linker is attached to the N-terminal IQ-end of the IQN36 sequence.

The linker is selected from the group of an antibody-antibody complex, antibody-antigen complex or streptavidin-biotin system.

The ultra-violet excitable fluorophore is selected from the group of lanthanides and wherein the light emitting fluorophore matches the excitation wavelength of the selected lanthanide.

Preferably the light emitting fluorophore is allophycocyanin, more preferably streptavidin-allophycocyanin, and the ultra-violet excitable fluorophore is europium.

Other light emitting fluorophores are selected from the group of alexa fluor 546, rhodamine or Cy3 and wherein the ultra-violet excitable fluorophore is terbium.

Part of the invention is also a method for identifying a compound that interferes with the formation of the HIV six-helix bundle of gp41 comprising:
providing a first helical polypeptide consisting essentially of the sequence derived from the heptad-repeat 1 (HR1) region of Human Immunodeficiency Virus (HIV), preferably wherein said polypeptide consists essentially of the sequence of IQN36 (SEQ ID NO:1);
providing a second helical polypeptide consisting essentially of the sequence derived from the heptad-repeat 2 (HR2) region of HIV, preferably wherein said polypeptide consists essentially of the sequence of C34 (SEQ ID NO: 2);
wherein said IQN36 is labeled with a light emitting fluorophore and said C34 is labeled with an ultra-violet excitable fluorophore;
providing a test composition comprising the compound;
measuring the degree of complex formation between the first helical polypeptide and the second helical polypeptide in the presence of the test composition comprising the compound using fluorescence resonance energy transfer; and
comparing the measured degree of complex formation to the degree of complex formation between the first helical polypeptide and the second helical polypeptide in the absence of the test composition comprising the compound to identify the compound that interferes with the formation of the HIV six-helix bundle of gp41.

Said IQN36 sequence may comprise a linker between the label and the IQ-moiety of the IQN36 sequence, preferably the linker is attached to the N-terminal IQ-end of the IQN36 sequence.

The linker is selected from the group of an antibody-antibody complex, antibody-antigen complex or streptavidin-biotin system.

The ultra-violet excitable fluorophore is selected from the group of lanthanides and wherein the light emitting fluorophore matches the excitation wavelength of the selected lanthanide.

Preferably the light emitting fluorophore is allophycocyanin, more preferably streptavidin-allophycocyanin, and the ultra-violet excitable fluorophore is europium.

Other light emitting fluorophores for use in the method are selected from the group of alexa fluor 546, rhodamine or Cy3 and wherein the ultra-violet excitable fluorophore is terbium.

Another embodiment of the present invention is a method for identifying the mechanism of inhibition of the formation of the HIV six-helix bundle of gp41 comprising:
providing a first helical polypeptide consisting essentially of the sequence of IQN36 (SEQ ID NO:1);

providing a second helical polypeptide consisting essentially of the sequence of C34 (SEQ ID NO: 2) wherein said IQN36 is labeled with a light emitting fluorophore and said C34 is labeled with an ultra-violet excitable fluorophore;
providing a test composition comprising a compound that interferes with the formation of the six-helix bundle of gp41;
measuring the degree of complex formation between the first helical polypeptide and the second helical polypeptide in the presence of the test composition comprising a compound that interferes with the formation of the six-helix bundle of gp41 using fluorescence resonance energy transfer; and
comparing the measured degree of complex formation to the degree of complex formation between the first helical polypeptide and the second helical polypeptide in the absence of the test composition comprising the compound that interferes with the formation of the six-helix bundle of gp41 to identify the mechanism of inhibition of the formation of the HIV six-helix bundle of gp41.

The compounds identified by the above-mentioned method are listed in the Example section of the present description and the thus identified compounds can be used for inhibiting the formation of the HIV six-helix bundle of gp41.

These terms as used herein are defined as follows:

Helical polypeptide as used herein refers to a polypeptide with a helical content of at least 70% in aqueous solution, such as for example 74%, 80%, 85%, 90% and 95%. The percent helical content is estimated as previously described (Sreerama et al., Anal. Biochem. 209:32-44 (1993)).

A fusion inhibitor, as used herein, is any compound that prevents membrane fusion between target cells and free virus or viral infected cells. For example, a HIV fusion inhibitor may be any compound that binds to gp41 and prevents the fusogenic six-helical bundle formation, thus decreases gp41-mediated membrane fusion. In one embodiment, a fusion inhibitor is any compound that decreases the degree of complex formation or binding affinity. In another embodiment, a fusion inhibitor is chosen from peptides, derivatized peptides, C-peptides, D-peptides, N-peptides, cyclic or linear, small and large molecules that decrease gp41-mediated membrane fusion, including, for example, disrupting the complex formation of the N- and C-helices of gp41.

C-peptides are peptide segments derived from the second heptad repeat region of HIV gp41 sequence and their derivatives, including C34, C28, T20, and T1249.

N-peptides are peptide segments derived from the first heptad repeat region of HIV gp41 sequence and their derivatives, including N36 and DP107.

A test composition comprises any compound, including, but not limited to, peptides, dipeptides, tripeptides, polypeptides, proteins, small and large organic molecules and derivatives thereof Large organic molecules are those with a molecular weight higher than 1000 Daltons.

Complex formation or binding affinity, as used herein, refers to the ability of at least two entities, for example, at least two peptides, to interact with one another, such as, for example, by hydrogen bonding and Van der Waals interactions. The degree of complex formation of two peptides would therefore be the extent of interaction between two peptides. This parameter ranges between 0-100%, with 100% being one peptide completely bound to the other peptide at the experimental concentrations.

The binding affinity of the first helical polypeptide and the second helical polypeptide, both alone and in the presence of the test composition, may be measured by any method known in the art. For example, the binding affinity may be measured by titrating the second helical polypeptide against a fixed concentration of the first helical polypeptide or vice versa.

The degree of complex formation measures the percentage of bound second helical polypeptide relative to the total amount of second helical polypeptide, at fixed concentrations of the first and second helical polypeptides. Although one can calculate binding affinity from the degree of complex formation, this is usually not recommended because of possible large errors. The difference between degree of complex formation and binding affinity is that binding affinity is usually determined by a series of measurements of degree of complex formation at a fixed concentration of one binding component, and at increasing concentrations of the second binding component until the first binding component is completely bound. A titration curve is thus obtained.

By the use of the phrase “consisting essentially of” in describing the sequences of the invention, it is meant to include any changes, variations, derivatives, additions, insertions, and mutations to the sequence of IQN36 and/or C34 that do not prohibit binding of the first helical polypeptide and the second helical polypeptide.

Peptides mentioned or used in the current description are:

SEQ ID NO 1 (HIV-IQN36)
RMKQIEDKIEEIESKQKKIENEIARIKKLISGIVQQQNNLLRAIE
AQQHLLQLTVWGIKQLQARIL
SEQ ID NO 2 (HIV-C34):
WMEWDREINNYTSLIHSLIEESQNQQEKNEQELL
SEQ ID NO 3 (HIV-IQN17):
RMKQIEDKIEEIESKQKKIENEIARIKKLLQLTVWGIKQLQARIL
SEQ ID NO 4: (RSV-C45):
NFYDPLVFPSDEFDASISQVNEKINQSLAFIRKSDELLHNVNAGK
SEQ ID NO 5 (T20):
YTSLIHSLIEESQNQQEKNEQELLELDKWASLWNWF

### EXAMPLES

Streptavidin-allophycocyanin (S-APC) was purchased from Perkin Elmer and peptides C34, T20, C45, biotin-labeled IQN36 (IQN36-biotin) and europium-labeled C34 (C34-eu) were synthesized according to standard protocols. All reagents were dissolved in 100 mM Hepes buffer pH 7.2. Test compounds were serially diluted and then added to 384-well plates. A final concentration of 10 nM S-APC and 100 nM IQN36-biotin were mixed in tubes, incubated for 30 min at room temperature and then added to well containing test compound. The compound/S-APC/IQN36-biotin mixture was incubated for another 2 hours at room temperature. After the incubation, C34-eu was added at a final concentration of 125 nM followed by a final incubation of the mixture for 30 min at room temperature. After the incubation, the fluorescence resonance energy transfer (FRET) signal was detected using a Viewlux reader and used to calculate the 50% inhibitory concentration (IC₅₀) of the test compound.

IC₅₀ is the drug concentration at which 50% of the FRET signal (or 6HB complex formation) is inhibited.

### Alternative Assay Set-Ups

The assays principle can be applied to a number of different labels and capture techniques.

Instead of the streptavidin-biotin system for peptide capture to the assay plate, antibodies targeting the peptides can be used.

The europium label can be substituted with other lanthanides such as a terbium label, which exhibit similar properties.

The APC label can be substituted for a number of different labels where the peak of the lanthanide emission matches the excitation wavelength of the label. The preferred matching pair is europium—APC. However, combinations such as (not limited to) terbium—Alexa Fluor 546, terbium—rhodamine and terbium—Cy3 are possible.

### Results

Two peptides with reported inhibitory activity against 6-helix bundle (HB) formation (T20 and C34) and an irrelevant negative control peptide derived from the RSV F-protein (C45) were used to validate the method. T20 and C34 showed inhibition of the 6-HB formation and FRET signal whereas C45 showed no inhibitory activity up to 10 μM.

| Peptide | IC₅₀ (μM) |
|---|---|
| T20 | 1 ± 0.5 |
| C34 | 0.07 ± 0.04 |
| C45 | >10 |

The well-established fusion inhibitors C34 and T20 exhibit potent activity in this test system. Diverse compound libraries in a high-throughput screening campaign were evaluated in order to identify several inhibitors falling into different chemical classes with low micromolar IC₅₀. Among those were compounds that proved to be active in a cell-based antiviral assay, in the absence of cytotoxicity. (Table 1)

Validation of the assay was done with various unlabeled HR2-peptides and small molecules with putative 6-helix bundle inhibitory activity. Different FRET couples were tested, quenching issues were addressed, and the assay was optimized for high-throughput screening (HTS). Under optimal conditions, the assay proved to have a convenient dynamic range and low signal measurement-associated data variation (z-factor=0.8, signal/noise>10). The test system according to the invention was found to be selective towards HIV-1 fusion inhibitors, and when comparing several parallel experiments, data were reproducible. Evaluation of diverse in-house libraries in a HTS campaign, allowed identifying different chemical scaffolds that interact with this molecular mechanism.

TABLE 1

| Compound | | HIV-FRET-IQN36_C34 |
|---|---|---|
| No | Structure | IC₅₀ (μM) |
| 1 | 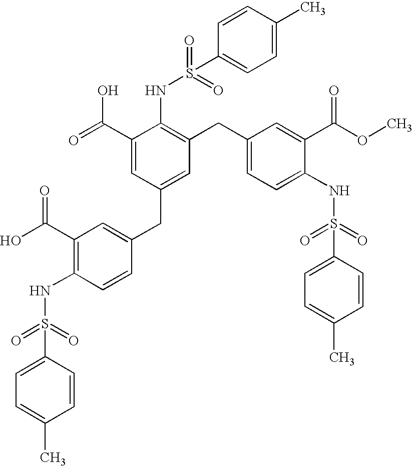 | 3.27 |
| 2 | 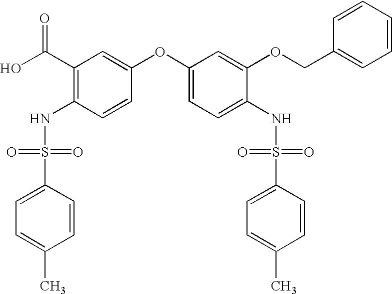 | 2.72 |
| 3 | 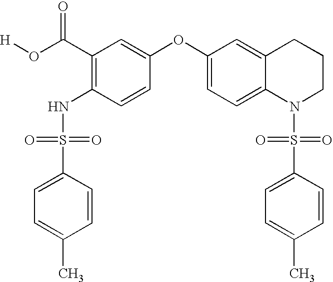 | 2.88 |
| 4 | 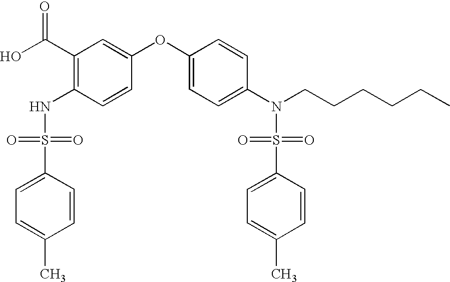 | 4.45 |
| 5 | 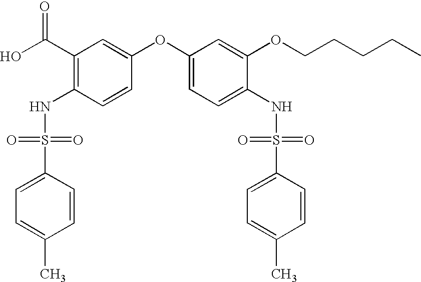 | 3.48 |
| 6 | 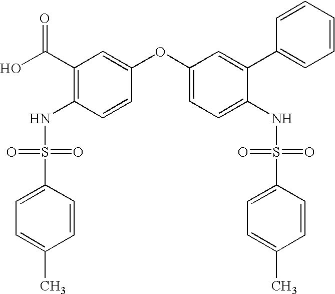 | 3.16 |
| 7 | 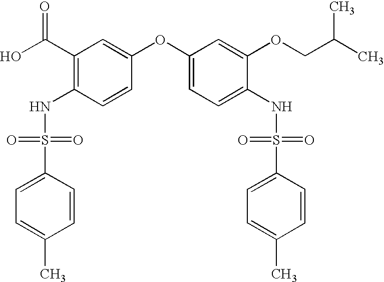 | 3.27 |
| 8 | 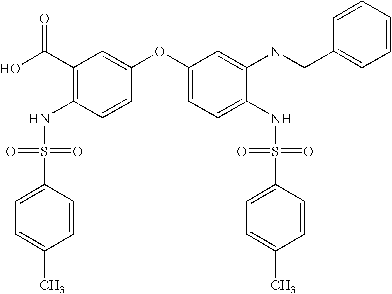 | 2.27 |
| 9 | 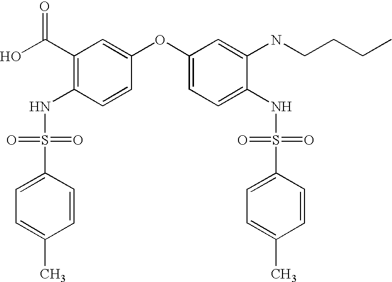 | 1.81 |
| 10 | 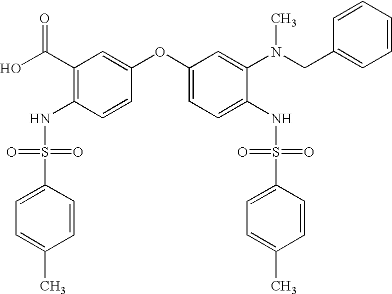 | 2.64 |
| 11 | 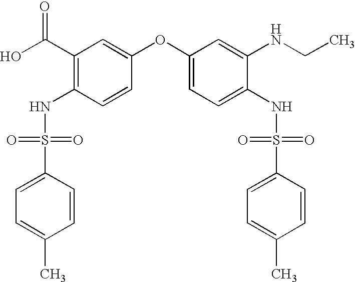 | 2.47 |
| 12 | 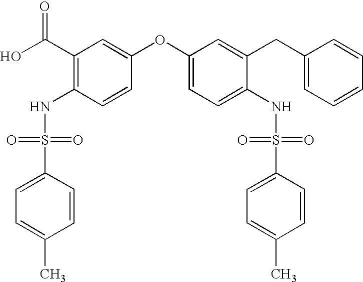 | 2.09 |
| 13 | 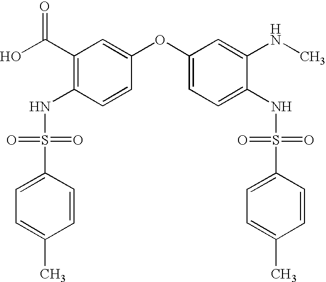 | 2.69 |

### Preparation of Compounds (2-13) as Mentioned in Table 1 Compound 2

### Preparation of 2-Benzyloxy-4-fluoro-1-nitro-benzene

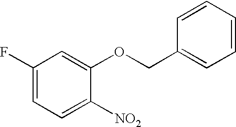

Dissolved the commercially available 5-fluoro-2-nitrophenol (250 mg, 1.59 mmol) in 2-butanone (10 ml) under Nitrogen. Added K₂CO₃ (659 mg, 4.77 mmol) and benzylbromide (272 mg, 1.59 mmol). Heated to 80° C. for 1 hour. Added a catalytic amount of KI. Stirred for another 2.5 hours. Cooled to room temperature and left standing for 18 hours. Filtered off and washed with 2-butanone. The filtrate was concentrated in vacuo. Added EtOAc, washed with 0.5 N NaOH (2×25 ml) and aq. sat. NaCl (25 ml). Dried over Na₂SO₄, filtered off and concentrated in vacuo. Gave 371 mg (y:94%) slightly yellow solid.

### Preparation of 5-(3-Benzyloxy-4-nitrophenoxy)-2-(toluene-4-sulfonylamino)benzoic acid methyl ester

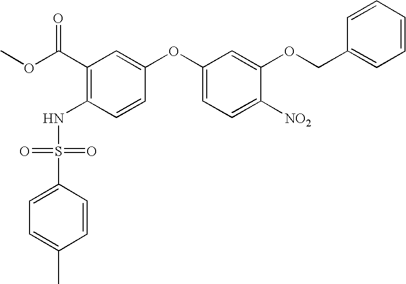

Dissolved 5-Hydroxy-2-(toluene-4-sulfonylamino)-benzoic acid methyl ester (200 mg, 0.622 moll) in dry DMF (5 ml) under Argon. Added K₂CO₃ (215 mg, 1.56 mmol) and 2-Benzyloxy-4-fluoro-1-nitro-benzene (161 mg, 0.653 mmol). Heated to 80° C. for 4 hours. Cooled to room temperature. Added EtOAc, washed with 0.5 M KHSO₄ (2×25 ml), aq. sat. NH₄Cl (25 ml), 0.5 M NaOH (2×25 ml) and aq. sat. NaCl (25 ml). Dried over Na₂SO₄, filtered off and concentrated in vacuo. Stirred up in EtOH. Filtered the crystals off and washed with EtOH, dried on filter. Gave 245 mg (y:71%) off white solid.

LC: 90%

MS: [M+H]⁺=549

GC: >95%

MS: [M]⁺=141/91

### Preparation of 5-(4-Amino-3-benzyloxy-phenoxy)-2-(toluene-4-sulfonylamino)-benzoic acid methyl ester

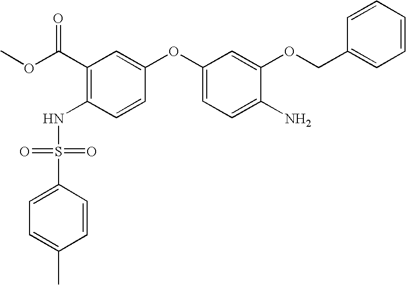

Dissolved NH₄Cl (119 mg, 2.23 mmol) in water (2 ml). Added Fe (125 mg, 2.23 mmol) followed by a solution of 5-(3-Benzyloxy-4-nitro-phenoxy)-2-(toluene-4-sulfonyl-amino)-benzoic acid methyl ester (245 mg, 0.447 mmol) in a mixture of 1:1 MeOH/water (10 ml). Heated to 70° C. under Nitrogen for 3.5 hours. Cooled to room temperature. Filtered off over kieselguhr, rinsed with EtOAc. Washed the filtrate with aq. sat.NaHCO₃ (25 ml) and aq. sat. NaCl (25 ml). Dried over Na₂SO₄, filtered off and concentrated in vacuo.

Preparative plate (2 mm layer thickness), eluens Heptane:EtOAc 1:1. Scraped the most UV intense band of the plate. Removed the product from silica gel by stirring up in CH₂Cl₂:MeOH 9:1. Filtered off, washed and concentrated in vacuo, stripped with CH₂Cl₂ (2×). Gave 190 mg (y:82%) yellow solid

LC: 87%

MS: [M+H]+=519

### Preparation of 5-[3-Benzyloxy-4-(toluene-4-sulfonylamino)-phenoxy]-2-(toluene-4-sulfonylamino)-benzoic acid methyl ester

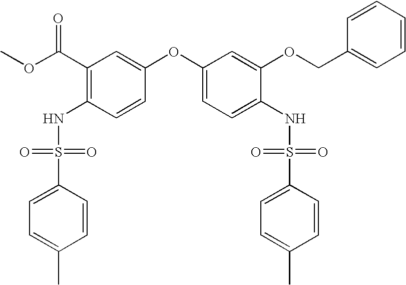

Dissolved 5-(4-Amino-3-benzyloxy-phenoxy)-2-(toluene-4-sulfonylamino)-benzoic acid methyl ester (190 mg, 0.366 mmol) in CH₂Cl₂ (10 ml) under Nitrogen. Added pyridine (60 μl, 0.732 mmol) and p-toluene-sulfonyl chloride (77 mg, 0.403 mmol). Stirred at room temperature for 18 hours. Added CH₂Cl₂ and washed with 0.5N HCl (25 ml), 0.5N NaOH (25 ml) and aq. sat. NaCl (25 ml). Dried over Na₂SO₄, filtered off and concentrated in vacuo.

Coated the crude product on isolute (0.6 g). Flash column, eluens 10% to 25% EtOAc in Heptane. The pure fractions were concentrated in vacuo, stripped with CH₂Cl₂ and dried on vacuo line. Gave 188 mg (76%) product.

LC: 87%

MS: [M−1]+=671

### Preparation of 5-[3-Benzyloxy-4-(toluene-4-sulfonylamino)-phenoxy]-2-(toluene-4-sulfonylamino)-benzoic acid

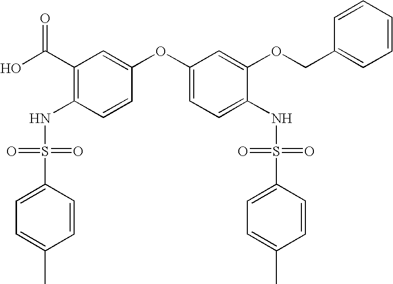

Dissolved 5-[3-Benzyloxy-4-(toluene-4-sulfonylamino)-phenoxy]-2-(toluene-4-sulfonylamino)-benzoic acid methyl ester (188 mg, 0.279 mmol) in a mixture of 1:1 water/THF (10 ml). Added LiOH (58 mg, 1.40 mmol) and stirred at 50° C. for 5 hours. Distilled the THF off. Acidified with 2N HCl, a precipitate formed, filtered off and washed with water. Dried in vacuo at 40° C. overnight. The solid was stirred up in diisopropyl ether. Filtered off and washed with diisopropyl ether. Dried in vacuo at 40° C. for 5 hours. Gave 131 mg (y: 71%) product.

LC: >95%

MS: [M−1]⁺=657

NMR:

¹H (400 MHz, dmso-d6)

δ 9.46 (s, 1H), 7.67 (d, J=8.32 Hz, 2H), 7.51-7.47 (m, 3H), 7.36 (d, 8.32 Hz, 2H), 7.30-7.28 (m, 4H), 7.20-7.14 (m, 6H), 6.59 (d, J=2.28 Hz, 1H), 6.45 (dd, J=2.56 Hz, J=8.60Hz, 1H), 4.77 (s, 2H), 2.31 (d, J=10.8 Hz, 6H)

### Compound 3

### Preparation of 5-Chloro-2-nitro-benzoic acid methyl ester

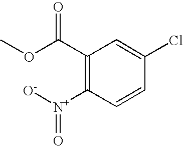

5-Chloro-2-nitro-benzoic acid (50 g, 247.5 mmol) was dissolved in methanol (700 ml), and thionylchloride was added slowly (12.65 ml, 173.3 mmol). The solution was refluxed for 40 hours. The solvent was evaporated under reduced pressure. The residue was dissolved in dichloromethane, washed with a sodium bicarbonate-solution, dried over sodium sulfate and evaporated under reduced pressure. The crude product was used in the next step without further purification (42.94 g, 80.46%).

MS: [M+H]⁺=216

### Preparation of 2-Nitro-5-(quinolin-6-yloxy)-benzoic acid methyl ester

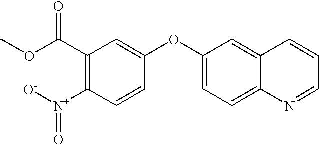

5-Chloro-2-nitro-benzoic acid methyl ester (2593.7 mg, 12.031 mmol), 6-hydroxy-quinoline (1782 mg, 12.031 mmol) and sodium carbonate were dissolved in DMF (100 ml). The solution was heated at 120° C. for 16 hours. The solution was filtered and evaporated under reduced pressure. The residue was purified by column chromato-graphy (SiO₂) on elution with dichloromethane: methanol 100:0 to 99:1 (2467.7 mg, 57%).

MS: [M+30]⁺=324

### Preparation of 2-Amino-5-(1,2,3,4-tetrahydro-quinolin-6-yloxy)-benzoic acid methyl ester

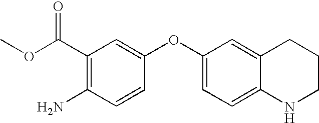

2-Nitro-5-(quinolin-6-yloxy)-benzoic acid methyl ester (2.4677 g, 7.6 mmol) was dissolved in methanol (50 ml). 10% Palladium on carbon (300 mg, 282 μmol) and 6N HCl in isopropanol (1.5 ml, 37 mmol) were added and the dispersion was hydrogenated for 18 hours. The catalyst was filtered off and the solvent was evaporated under reduced pressure. The crude product was used in the next step without further purification (2.477 g, 99%).

MS: [M+H]⁺=299

### Preparation of 2-(4-methyl phenyl sulfonamino)-5-[1-(toluene-4-sulfonyl)-1,2,3,4-tetrahydro-quinolin-6-yloxy]-benzoic acid methyl ester

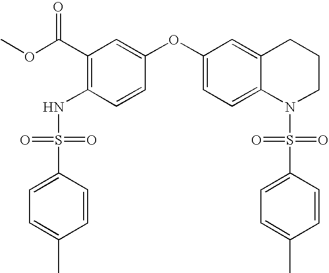

2-Amino-5-(1,2,3,4-tetrahydro-quinolin-6-yloxy)-benzoic acid methyl ester (1 g, 3.352 mmol) and p-toluenesulfonyl chloride (1.917 g, 10.05 mmol) were dissolved in pyridine (5 ml). The solution was stirred at room temperature for 64 hours. The solvent was evaporated under reduced pressure. The residue was purified by column chromatography (SiO₂) on elution with dichloromethane (200 mg, 9.8%).

MS: [M+17]⁺=623

### Preparation of 2-(4-methyl phenyl sulfonamino)-5-[1-(toluene-4-sulfonyl)-1,2,3,4-tetrahydro-quinolin-6-yloxy]-benzoic acid

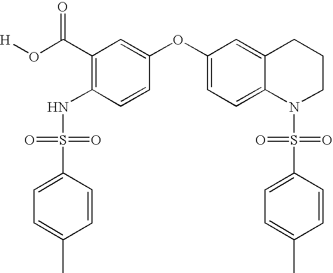

2-(4-methyl phenyl sulfonamino)-5-[1-(toluene-4-sulfonyl)-1,2,3,4-tetrahydro-quinolin-6-yloxy]-benzoic acid methyl ester (200 mg, 330 μmol) was dissolved in tetrahydrofurane (50 ml). A solution of lithium hydroxide monohydrate in water (50 ml, 0.4 M) was added to the tetrahydrofurane solution, resulting in an overall concentration of 0.2 M of lithium hydroxide. The solution was stirred at room temperature for 24 hours. The solvents were evaporated under reduced pressure. The residue was extracted with ethyl acetate/water, acidified with HCl until pH 7. The organic layer was dried over magnesium sulfate and evaporated under reduced pressure to obtain the desired product (114.6 mg, 57%, purity (LC)=97.3%).

MS: [M−H]⁻=635

### Compound 4

### Preparation of 5-Hydroxy-2-nitro-benzoic acid methyl ester

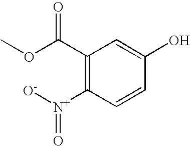

5-Hydroxy-2-nitro-benzoic acid (20 g, 109 mmol) was dissolved in methanol (700 ml) and thionylchloride (5.6 ml, 76 mmol) was slowly added. The solution was refluxed for 40 hours. The solvent was evaporated under reduced pressure, and the mixture was dissolved in ethyl acetate. The organic layer was washed with brine, dried over sodium sulfate and evaporated under reduced pressure. The crude product was used in the next step without further purification (21.5 g, 97.5%).

MS: [M−H]⁻=196

### Preparation of 2-Nitro-5-(4-nitro-phenoxy)-benzoic acid methyl ester

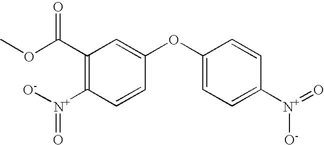

5-Hydroxy-2-nitrobenzoic acid methyl ester (10 g, 51 mmol), 1-fluoro-4-nitrobenzene (7.2 g, 51 mmol), and potassium carbonate (8.4 g, 61 mmol) were dissolved in acetonitrile (700 ml). The solution was refluxed for 40 hours. The mixture was filtered and evaporated under reduced pressure. The residue was purified by column chromatography (SiO₂) on elution with dichloromethane:heptane (50:50) (3.7 g, 23%).

MS: [M+H]⁺=319

### Preparation of 2-amino-5-(4-amino-phenoxy)-benzoic acid methyl ester

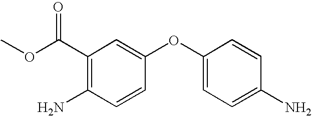

2-Nitro-5-(4-nitro-phenoxy)-benzoic acid methyl ester (3.70 g, 12 mmol) was dissolved in methanol (200 ml). 10% Palladium on carbon (300 mg, 282 μmol) was added and the dispersion was hydrogenated for 18 hours. The catalyst was filtered off and the solvent was evaporated under reduced pressure. The crude product was used in the next step without further purification (2.93 g, 92%).

MS: [M+H]⁺=259

### Preparation of 2-Amino-5-(4-hexylamino-phenoxy)-benzoic acid methyl ester

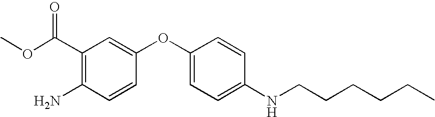

2-Amino-5-(4-amino-phenoxy)-benzoic acid methyl ester (191 mg, 740 μmol), hexanal (59.3 mg, 592 μmol) and sodium triacetoxyborohydride (157 mg, 740 μmol) were dissolved in dichloromethane (20 ml). The solution was stirred at room temperature for 16 hours. The solution was diluted with water. The organic layer was washed with water, dried over magnesium sulfate and evaporated under reduced pressure. The residue was purified by column chromatography (SiO₂) on elution with dichloromethane (71.6 mg, 17.3%).

MS: [M+H]⁺=343

### Preparation of 5-{4-[Hexyl-(toluene-4-sulfonyl)-amino]-phenoxy}-2-(4-methyl phenyl sulfonamino)-benzoic acid methyl ester

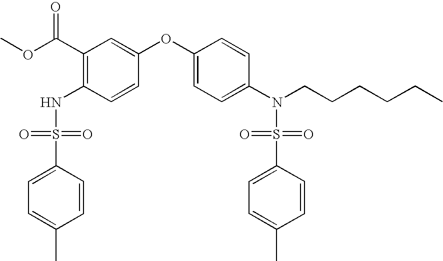

2-Amino-5-(4-hexylamino-phenoxy)-benzoic acid methyl ester (71.6 mg, 209 μmol) and p-toluenesulfonyl chloride (92 mg, 481 μmol) were dissolved in pyridine (2 ml).

The solution was stirred at room temperature for 22 hours. The solvent was evaporated under reduced pressure and the residue was purified by column chromatography (SiO₂) on elution with dichloromethane (30 mg, 22%).

MS: [M+17]⁺=667

### Preparation of 5-{4-[Hexyl-(toluene-4-sulfonyl)-amino]-phenoxy}-2-(4-methyl phenyl sulfonamino)-benzoic acid

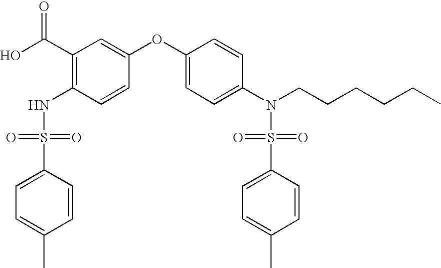

5-{4-[Hexyl-(toluene-4-sulfonyl)-amino]-phenoxy}-2-(4-methyl phenyl sulfonamino)-benzoic acid methyl ester (30 mg, 46 μmol) was dissolved in tetrahydrofurane (10 ml). A solution of lithium hydroxide monohydrate in water (10 ml, 0.4 M) was added to the tetrahydrofurane solution, resulting in an overall concentration of 0.2 M of lithium hydroxide. The solution was stirred at room temperature for 64 hours. The solvents were evaporated under reduced pressure. The residue was extracted with ethyl acetate/water, acidified with HCl until pH 7. The organic layer was dried over magnesium sulfate and evaporated under reduced pressure. The residue was purified by column chromatography (SiO₂) on elution with ethyl acetate:heptane 3:7 to 9:1 (25.5 mg, 78%, purity (LC)=90%).

MS: [M−H]⁻=635

### Compound 5

### Preparation of 4-fluoro-2-pentoxy-1-nitro-benzene

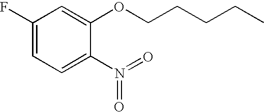

Dissolved commercially available 5-fluoro-2-nitrophenol (250 mg, 1.59 mmol) in 2-butanone (10 ml) under Nitrogen. Added K₂CO₃ (659 mg, 4.77 mmol). Added 1-iodo-pentane (331 mg, 1.67 mmol) as a solution in 2-butanone (2 ml). Stirred at 80° C. for 18 hours. Cooled to room temperature, filtered off and washed with 2-butanone. The filtrate was concentrated in vacuo. Redissolved in EtOAc and washed with 0.5N NaOH (2×25 ml) and aq. sat. NaCl (25 ml). Dried over Na₂SO₄, filtered off and concentrated in vacuo. Gave 279 mg (y: 77%) yellow liquid.

GC: >95%

MS: [M]⁺=227/157

### Preparation of 5-(4-nitro-3-pentyloxy-phenoxy)-2-(toluene-4-sulfonylamino)-benzoic acid methyl ester

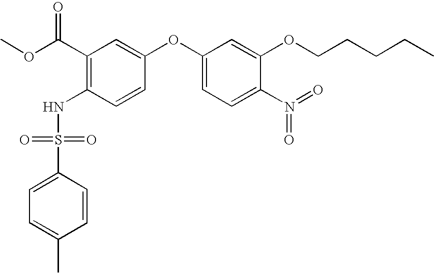

A suspension of methyl 5-hydroxy-2-[(4-methylphenyl)sulfonyl]aminobenzoate (395 mg, 1.23 mmol), 4-fluoro-2-pentoxy-1-nitro-benzene (279 mg, 1.23 mmol) and anh. potassium carbonate (424 mg, 3.07 mmol) in DMF (10 mL) was stirred at 80° C. After 4 hours, the reaction was diluted with ethyl acetate (150 mL) and washed with aq. 0.5N KHSO₄ (2×100 mL), aqueous saturated ammonium chloride (100 mL), aq. 0.5N NaOH (100 mL) and brine (2×100 mL). The organic layer was dried over anhydrous sodium sulfate and evaporated in vacuo. The oily residue was stirred in ethanol (50 mL) for 0.5 hours and precipitation of a white solid occurred. The suspension was stirred 0.5 hours at 0° C. and filtered. The white solid was dried in vacuo at 40° C. for 16 hours to afford the desired product (358 mg, 55%, purity n.d.).

### Preparation of 5-(4-amino-3-pentyloxy-phenoxy)-2-(toluene-4-sulfonylamino)-benzoic acid methyl ester

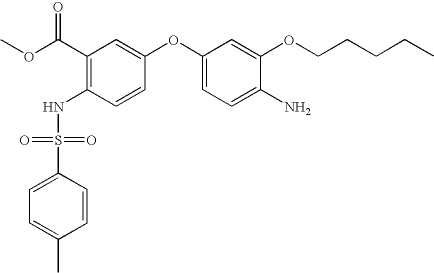

A suspension of 5-(4-nitro-3-pentyloxy-phenoxy)-2-(toluene-4-sulfonylamino)-benzoic acid methyl ester (358 mg, 0.68 mmol) and 10% palladium on activated carbon (36 mg, 0.034 mmol) in tetrahydrofuran (5 mL) and methanol (5 mL) was stirred at room temperature under 1 atmosphere of hydrogen. After 4 hours the reaction was filtered over Kieselguhr and evaporated under reduced pressure to afford the desired product (430 mg, 99%, purity n.d.)

### Preparation of 5-[3-pentyloxy-4-(toluene-4-sulfonylamino)-phenoxy]-2-(toluene-4-sulfonylamino)-benzoic acid methyl ester

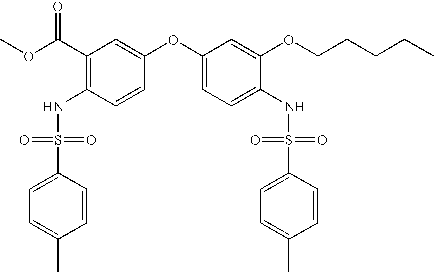

A solution of 5-(4-amino-3-pentyloxy-phenoxy)-2-(toluene-4-sulfonylamino)-benzoic acid methyl ester (430 mg, 0.86 mmol), p-toluenesulfonylchloride (196 mg, 1.03 mmol) and pyridine (136 mg, 2.81 mmol) in dichloromethane (10 mL) was stirred for 72 hours at room temperature. The reaction was diluted with dichloromethane (50 mL) and washed with aqueous 0.5N potassium hydrogen sulfate (30 mL), aq. saturated sodium hydrogen carbonate (30 mL) and dried over anhydrous sodium sulfate. After concentration under reduced pressure, the residue was triturated in boiling DIPE containing 5% ethanol. After cooling to 0° C., the crystals were filtered and dried in vacuo for 16 hours (300 mg, 53%, purity (LC)>95%).

MS: [M−H]−=651

NMR ¹H (DMSO-d6): δ 10.05 (bs, 1H), 9.29 (bs, 1H), 7.61 (d, J=8.36 Hz, 2H), 7.50 (d, J=8.32 Hz, 2H), 7.42 (d, J=8.60 Hz, 1H), 7.35 (d, J=8.08 Hz, 2H), 7.30 (d, J=8.08 Hz, 2H), 7.26-7.21 (m, 2H), 7.21 (d, J=8.56 Hz, 1H) 6.59 (d, J=2.52 Hz, 1H), 6.44 (dd, J=2.28 Hz and J=8.36 Hz, 1H), 3.55 (t, J=6.56 Hz, 2H), 2.35 (s, 3H), 2.34 (s, 3H), 1.42-1.35 (m, 2H), 1.27-1.13 (m, 4H) 0.86 (t, J=7.06 Hz, 3H)

### Preparation of 5-[3-pentyloxy-4-(toluene-4-sulfonylamino)-phenoxy]-2-(toluene-4-sulfonylamino)-benzoic acid

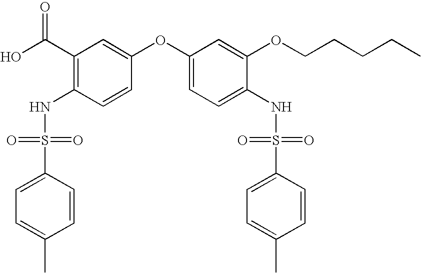

A mixture of 5-[3-pentyloxy-4-(toluene-4-sulfonylamino)phenoxy]-2-(toluene-4-sulfonylamino)-benzoic acid methyl ester (150 mg, 0.23 mmol), LiOH (39 mg, 0.94 mmol), water (2 mL) and THF (2 mL) was stirred for 16 hours at room temperature and for 1 hour at 60° C. The reaction was acidified to pH=1 with aq. 2N HCl and the THF was evaporated under reduced pressure. The suspension was extracted with DCM (2×50 mL) and the extracts were dried over anhydrous sodium sulfate. Concentration of the organic solution afforded the desired compound (142 mg, 97%, purity (LC)>95%).

MS: [M−H]⁻=609

NMR: ¹H (DMSO-d6): δ 10.86 (bs, 1H), 9.26 (s, 1H), 7.66 (d, J=8.32 Hz, 2H), 7.53 (d, J=9.08 Hz, 1H), 7.48 (d, J=8.36 Hz, 2H), 7.36 (d, J=8.08 Hz, 2H), 7.33 (d, J=3.04 Hz, 1H), 7.29 (d, J=8.32 Hz, 2H), 7.25 (dd, J=3.04 Hz and 9.12 Hz, 1H), 7.21 (d, J=8.56 Hz, 1H), 6.58 (d, J=2.52, 1H), 6.44 (dd, J=2.52 Hz and 8.60 Hz, 1H), 3.53 (t, J=6.70 Hz, 2H), 2.34 (s, 3H), 2.34 (s, 3H), 1.40-1.33 (m, 2H), 1.26-1.14 (m, 4H), 0.86 (t, J=7.08 Hz, 3H)

### Compound 6

### Preparation of 4-fluoro-1-nitro-2-phenylbenzene

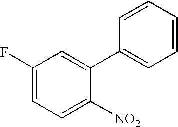

At RT and under N₂, Pd(PPh₃)₄ (114 mg, 0.098 mmol) was added to a mixture of 2-bromo-4-fluoro-1-nitrobenzene (539 mg, 2.45 mmol), phenylboronic acid (364 mg, 2.94 mmol), and sodium carbonate (1.04 g, 9.80 mmol) in DMSO (degassed by freeze-pump-thaw, 25 mL). The reaction was stirred at 80° C. for 16 hours and then water (10 mL, degassed by purging) was added. The reaction was continued for 24 hours. The reaction mixture was filtered over kiezelguhr. The filtrate was diluted with aq. sat. NaHCO₃ (100 mL) and extracted with EtOAc (2×50 mL). The EtOAc-extracts were washed with brine (1×50 mL). The combined EtOAc-extracts were dried (Na₂SO₄), filtered, and evaporated to dryness in vacuo. The crude product was purified by column chromatography (EtOAc/heptane: 1/100 to 1/20) to obtain 4-fluoro-1-nitro-2-phenylbenzene as a yellow oil (439 mg, 82%, purity (GC)>95%).

MS: [M]⁺=217.

### Preparation of 5-(6-nitro-biphenyl-3-yloxy)-2-(toluene-4-sulfonylamino)-benzoic acid methyl ester

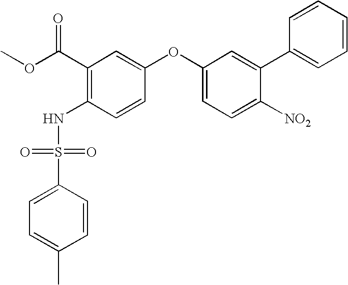

A mixture of methyl 5-hydroxy-2-[(4-methylphenyl)sulfonyl]aminobenzoate (653 mg mg, 2.03 mmol), 4-fluoro-1-nitro-2-phenylbenzene (440 mg, 2.03 mmol), and potassium carbonate (550 mg, 3.98 mmol) in DMF (10 mL) was stirred at 80° C. for 5 hours. The reaction was cooled to RT, diluted with EtOAc (50 mL) and washed with aq. 0.5 N KHSO₄ (1×50 mL), aq. sat. NaHCO₃ (1×50 mL), and brine (1×50 mL). The water layers were extracted with EtOAc (2×50 mL). The combined EtOAc-layers were dried (Na₂SO₄), filtered, and evaporated to dryness in vacuo. The crude product was recrystallized twice from EtOH to obtain 5-(6-nitro-biphenyl-3-yloxy)-2-(toluene-4-sulfonylamino)-benzoic acid methyl ester as a yellow solid (440 mg, 42%, purity (LC)>95%).

MS: [M−H]⁻=517.

### Preparation of 5-(6-Amino-biphenyl-3-yloxy)-2-(toluene-4-sulfonylamino)-benzoic acid methyl ester

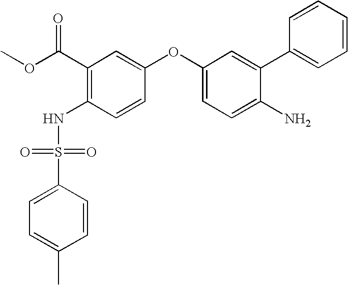

10% Pd-C (50 mg) was added to a solution of 5-(6-nitro-biphenyl-3-yloxy)-2-(toluene-4-sulfonylamino)-benzoic acid methyl ester (440 mg, 0.939 mmol) in THF/MeOH:1/1 (10 mL). The reaction was placed under 1 atmosphere of hydrogen and stirred at RT for 20 hours. The reaction mixture was filtered over Kiezelguhr and evaporated to dryness in vacuo. The crude product was purified by flash column chromatography (EtOAc/heptane: 1/4 to 1/3) to afford 5-(6-Amino-biphenyl-3-yloxy)-2-(toluene-4-sulfonyl-amino)benzoic acid methyl ester as a white foam (320 mg, 70%, purity (LC)>95%).

MS: [M−H]⁻=487.

### Preparation of 2-(Toluene-4-sulfonylamino)-5-[6-(toluene-4-sulfonylamino)-biphenyl-3-yloxy]-benzoic acid methyl ester

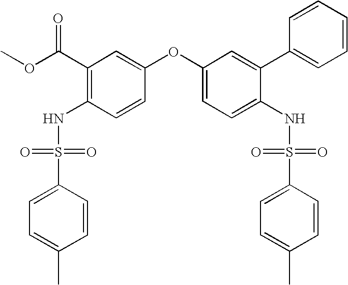

A solution of 5-(6-amino-biphenyl-3-yloxy)-2-(toluene-4-sulfonylamino)-benzoic acid methyl ester (316 mg, 0.717 mmol), tosyl chloride (205 mg, 1.08 mmol), and pyridine (116 mg, 1.47 mmol) in CH₂Cl₂ (5 mL) was stirred at 40° C. for 23 hours. The reaction was diluted with DCM (25 mL) and washed with aq. 0.5 N KHSO₄ (50 mL) and aq. sat. NaHCO₃ (50 mL). The aqueous-layers were extracted with DCM (1×25 mL). The combined DCM-layers were dried (Na₂SO₄), filtered and evaporated to dryness in vacuo. The crude product was purified by flash column chromatography (EtOAc/heptane: 1/4 to 1/1) to obtain the product as a white foam. The product was crystallized from EtOAc/heptane to afford 2-(toluene-4-sulfonylamino)-5-[6-(toluene-4-sulfonylamino)-biphenyl-3-yloxy]-benzoic acid methyl ester as small white crystals (335 mg, 73%, purity (LC)>95%).

MS: [M−H]⁻=641.

NMR: ¹H (CDCl₃): δ 10.27 (s, 1H), 7.71-7.63 (m, 4H), 7.53 (d, J=2.76 Hz, 1H), 7.43-7.27 (m, 5H), 7.22-7.17 (m, 4H), 7.13 (dd, J=2.76 Hz, and J=9.08 Hz), 6.89 (dd, J=2.76 Hz and J=8.80 Hz, 1H), 6.79-6.75 (m, 2H), 6.66 (d, J=2.80 Hz, 1H), 3.82 (s, 3H), 2.42 (s, 3H), 2.36 (s, 3H).

### Preparation of 2-(Toluene-4-sulfonylamino)-5-[6-(toluene-4-sulfonylamino)-biphenyl-3-yloxy]-benzoic acid

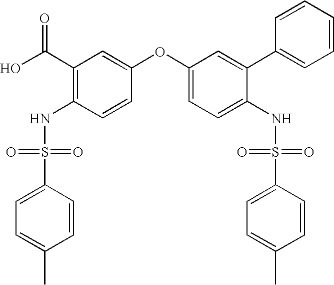

A mixture of 2-(Toluene-4-sulfonylamino)-5-[6-(toluene-4-sulfonylamino)-biphenyl-3-yloxy]-benzoic acid methyl ester (257 mg, 0.400 mmol) in aq. 0.5 M LiOH (5 mL) and THF (5 mL) was stirred at 60° C. for 4 hours and then at RT for 16 hours. The reaction was evaporated in vacuo to remove THF. The residue was acidified with aq. 2 M HCl (5 mL) and then diluted with water (25 mL). The aqueous suspension was extracted with DCM (2×10 mL). The combined DCM-extracts were dried (Na₂SO₄), filtered, and evaporated to dryness to the desired product as a white foam (251 mg, 100%, purity (LC)>95%).

MS: [M−H]⁻=627.

NMR: ¹H (DMSO-d6): δ 10.92 (bs, 1H), 9.42 (s, 1H), 7.63 (d, J=8.32 Hz, 2H), 7.53 (d, J=8.84 Hz, 1H), 7.42 (d, J=3.04 Hz, 1H), 7.37 (d, J=8.32 Hz, 2H), 7.34-7.29 (m, 5H), 7.24 (d, J=8.32 Hz, 2H), 7.19-7.16 (m, 2H), 7.00 (d, J=8.84 Hz, 1H), 6.88 (dd, J=3.04 Hz, 8.60 Hz, 1H), 6.78 (d, J=3.04 Hz, 1H), 2.36 (s, 3H), 2.31 (s, 3H).

### Compound 7

### Preparation of 4-Fluoro-2-isobutoxy-1-nitro-benzene

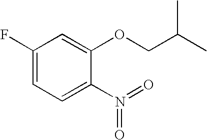

Dissolved commercially available 5-fluoro-2-nitrophenol (250 mg, 1.59 mmol) in 2-butanone (10 ml). Added K₂CO₃ (659 mg, 4.77 mmol). Added isobutyl iodine (410 mg, 2.22 mmol). Heated to 80° C. under Nitrogen for 20 hours. Left standing at room temperature for 48 hours. Decanted the solution, washed the solid with 2-butanone and concentrated the solution in vacuo. Added EtOAc, washed with 0.5 N NaOH (3×25 ml) and aq. sat. NaCl (25 ml). Dried over Na₂SO₄, filtered off and concentrated in vacuo. Gave 44 mg (13%) product.

GC: >95%

MS: [M]+=213/157

### Preparation of 5-(3-Isobutoxy-4-nitro-phenoxy)-2-(toluene-4-sulfonylamino)-benzoic acid methyl ester

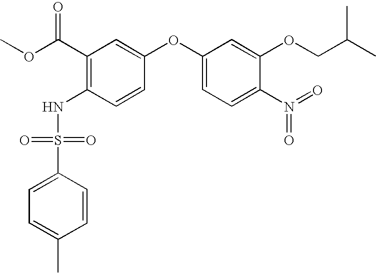

Dissolved 5-Hydroxy-2-(toluene-4-sulfonylamino)-benzoic acid methyl ester (144 mg, 0.447 mmol) in dry DMF (5 ml) under Argon. Added K₂CO₃ (154 mg, 1.12 mmol) and 4-Fluoro-2-isobutoxy-1-nitro-benzene (100 mg, 0.469 mmol) as a solution in dry DMF (2 ml). Heated to 80° C. for 4 hours. Cooled to room temperature. Added EtOAc, washed with 0.5N KHSO₄ (2×30 ml), aq. sat. NH₄Cl (30 ml), 0.5N NaOH (2×25 ml) and aq. sat. NaCl (25 ml). Dried over Na₂SO₄, filtered off and concentrated in vacuo. Stirred up in ice cooled EtOH, filtered off and washed with EtOH. Dried in vacuo at 40° C. Gave 131 mg (y: 57%) product.

LC: >95%

MS: [M+H]+=515

### Preparation of 5-(4-Amino-3-isobutoxy-phenoxy)-2-(toluene-4-sulfonylamino)-benzoic acid methyl ester

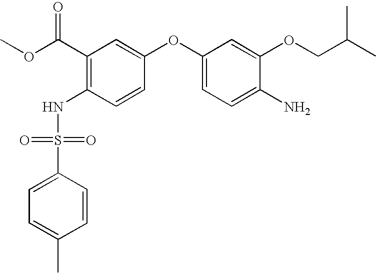

Dissolved NH₄Cl (70 mg, 1.27 mmol) in water (2 ml). Added Fe (71 mg, 1.27 mmol) and subsequently a suspension of 5-(3-Isobutoxy-4-nitro-phenoxy)-2-(toluene-4-sulfonylamino)-benzoic acid methyl ester (131 mg, 0.255 mmol) in a mixture of 1:1 MeOH/THF (10 ml) under Nitrogen. Heated to 70° C. for 4 hours. Cooled to room temperature and filtered over kieselguhr, rinsed with EtOAc. The filtrate was concentrated in vacuo. Redissolved in EtOAc and washed with aq. sat. NaHCO₃ (25 ml) and aq. sat. NaCl (25 ml). Dried over Na₂SO₄, filtered off and concentrated in vacuo.

Preparative plate (2 mm layer thickness), eluens Heptane:EtOAc 1:1. Scraped the most UV intense band of the plate. Removed the product from silica gel by stirring up in CH₂Cl₂:MeOH 9:1. Filtered off, washed and concentrated in vacuo. Stripped with CH₂Cl₂ (2×). Gave 75 mg (y: 61%) product.

LC: 94%

MS: [M+H]+=485

### Preparation of 5-[3-Isobutoxy-4-(toluene-4-sulfonylamino)-phenoxy]-2-(toluene-4-sulfonylamino)-benzoic acid methyl ester

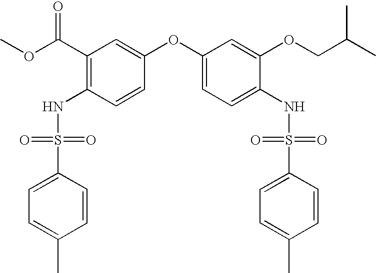

A solution of 5-(4-Amino-3-isobutoxy-phenoxy)-2-(toluene-4-sulfonylamino)-benzoic acid methyl ester (75 mg, 0.15 mmol), p-toluenesulfonylchloride (35 mg, 0.19 mmol) and pyridine (24 mg, 2.81 mmol) in dichloromethane (5 mL) was stirred for 72 hours at room temperature. The reaction was diluted with dichloromethane (50 mL) and washed with aqueous 0.5N potassium hydrogen sulfate (30 mL), saturated sodium hydrogen carbonate (30 mL) and dried over anhydrous sodium sulfate. The residue was purified with preperative TLC (Merck, 12 PLC-plates 20×20 cm silica gel 60 F₂₅₄, 2 mm with concentrating zone 20×4 cm), eluens: heptane (1)/EtOAc (1). The solid was dried in vacuo for 16 hours (59 mg, 62%, purity (LC)>95%).

MS: [M−H]⁻=637

### Preparation of 5-[3-Isobutoxy-4-(toluene-4-sulfonylamino)-phenoxy]-2-(toluene-4-sulfonylamino)-benzoic acid

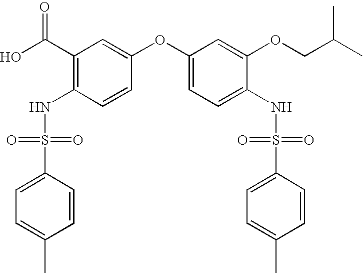

A mixture of 5-[3-isobutoxy-4-(toluene-4-sulfonylamino)-phenoxy]-2-(toluene-4-sulfonylamino)-benzoic acid methyl ester (59 mg, 0.09 mmol), LiOH (16 mg, 0.37 mmol), water (2 mL) and THF (2 mL) was stirred for 16 hours at room temperature. The reaction was acidified to pH=1 with aq. 2N HCl and the THF was evaporated under reduced pressure. The suspension was extracted with DCM (2×50 mL) and the extracts were dried over anhydrous sodium sulfate. Concentration of the organic solution afforded the crude product, which was purified with preperative TLC (Merck, 12 PLC-plates 20×20 cm silica gel 60 F₂₅₄, 2 mm with concentrating zone 20×4 cm), eluens: heptane (1)/EtOAc (1) containing 0.5% acetic acid. The solid was dried in vacuo for 16 hours (29 mg, 52%, purity (LC)>95%).

MS: [M−H]⁻=523

NMR: ¹H (DMSO-d6): δ 9.16 (s, 1H), 7.61 (d, J=8.08 Hz, 2H), 7.48 (d, J=8.08 Hz, 2H), 7.37-7.35 (m, 2H), 7.30 (d, J=8.08 Hz, 2H), 7.27 (d, J=8.08 Hz, 2H), 7.12 (d, J=8.56 Hz, 1H), 6.91 (dd, J=2.76 Hz and 8.84 Hz, 1H), 6.48 (d, J=2.24 Hz, 1H), 6.32 (dd, J=2.52 Hz and 8.84 Hz, 1H), 3.30 (d, J=6.56 Hz, 2H), 2.33 (s, 1H), 2.30 (s, 1H), 1.68 (m, 1H), 0.80 (s, 3H), 0.78 (s, 3H).

### Compound 8

### Preparation of N1-benzyl-5-fluoro-2-nitroaniline

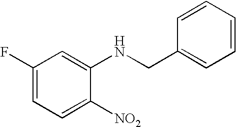

Under Argon and at RT, Pd(OAc)₂ (32 mg, 0.14 mmol) and BINAP (90 mg, 0.14 mmol) were added to a solution of benzylamine (225 mg, 2.10 mmol) and 4-fluoro-2-bromonitrobenzene (463 mg, 2.10 mmol) in toluene (degassed by purging, 5 mL). The reaction mixture was heated at 100° C. for 3 minutes and then cooled to 0° C. Upon addition of sodium tert-butoxide (253 mg, 2.63 mmol) the reaction mixture turned red. The reaction was stirred at 70° C. for 24 hours and then at RT for 48 hours. The reaction was filtered over Kiezelguhr. Residue was rinsed with EtOAc. The filtrate was evaporated to dryness in vacuo. The crude product was purified by flash column chromatography to afford N1-benzyl-5-fluoro-2-nitroaniline as a yellow crystalline compound (440 mg, 86%, purity (LC) n.d.).

NMR: ¹H (CDCl₃): δ 8.55 (bs, 1H), 8.24 (dd, J=6.04 Hz and J=9.32 Hz, 1H), 7.42-7.30 (m, 5H), 6.46 (dd, J=2.52 Hz and J=11.4 Hz, 1H), 6.39 (ddd, J=2.52 Hz and J=7.32 Hz and J=9.60 Hz, 1H), 2.12 (s, 2H).

### Preparation of 5-(3-benzylamino-4-nitro-phenoxy)-2-(toluene-4-sulfonylamino)-benzoic acid methyl ester

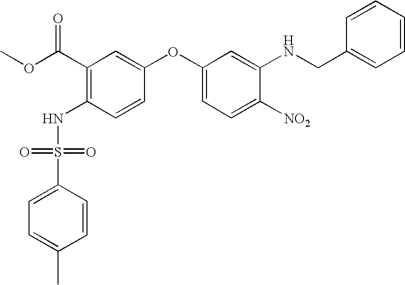

A mixture of methyl 5-hydroxy-2-[(4-methylphenyl)sulfonyl]aminobenzoate (431 mg, 1.34 mmol), N1-benzyl-5-fluoro-2-nitroaniline (324 mg, 1.32 mmol), and potassium carbonate (365 mg, 2.64 mmol) in DMF (10 mL) was stirred at 80° C. for 6 hours and then at RT for 12 hours. The reaction was diluted with brine (150 mL) and was extracted with EtOAc (75 mL). The EtOAc-extract was washed with aq. 0.5 M KHSO₄ (75 mL), aq. sat. NaHCO₃ (75 mL), and brine (75 mL). The aqueous-layers were extracted with EtOAc (75 mL). The combined EtOAc-extracts were dried (Na₂SO₄), filtered, and evaporated to dryness in vacuo. The crude product was purified by column chromatography (EtOAc/heptane: 1/6 to 1/4) to obtain the desired product as a yellow solid (611 mg, 83%, purity (LC)=85%).

MS: [M−H]⁻=546.

### Preparation of 5-(4-Amino-3-benzylamino-phenoxy)-2-(toluene-4-sulfonylamino)-benzoic acid methyl ester

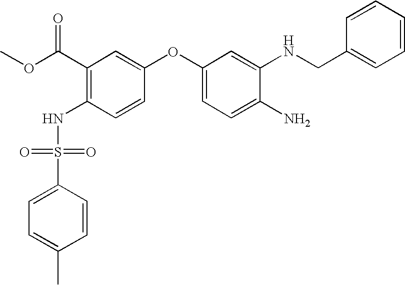

At RT and under nitrogen, a solution of 5-(3-benzylamino-4-nitro-phenoxy)-2-(toluene-4-sulfonylamino)-benzoic acid methyl ester (611 mg, 1.12 mmol) in THF/MeOH: 1/1 (15 mL) was added to a suspension of iron (312 mg, 5.59 mmol) and ammonium chloride (309 mg, 5.59 mmol) in water (10 mL). The reaction was heated at 70° C. for 2.5 h. The reaction was cooled to RT and then the reaction mixture was filtered over Kiezelguhr. The residue was rinsed with EtOAc (300 mL). The filtrate was washed with aq. sat. NaHCO₃ (75 mL) and brine (75 mL). The organic layer was dried (Na₂SO₄), filtered, and evaporated to dryness in vacuo. The crude product was purified by column chromatography (EtOAc/heptane: 1/3 to 1/2) to obtain the desired product (415 mg, 72%, purity (LC) n.d.).

NMR: ¹H (CDCl₃): δ 10.18 (s, 1H), 7.67 (d, J=8.32 Hz, 2H), 7.60 (d, J=9.08 Hz, 1H), 7.41 (d, J=2.76 Hz, 1H), 7.34-7.25 (m, 5H), 7.20 (d, J=8.08 Hz, 2H), 7.03 (dd, J=2.76 Hz and J=9.08 Hz, 1H), 6.67 (d, J=8.32 Hz, 1H), 6.28 (d, J=2.56 Hz, 1H), 6.23 (dd, J=8.32 Hz and J=2.56 Hz, 1H), 4.22 (s, 2H), 3.78 (s, 3H), 2.35 (s, 3H).

### Preparation of 5-[3-Benzylamino-4-(toluene-4-sulfonylamino)-phenoxy]-2-(toluene-4-sulfonylamino)-benzoic acid methyl ester

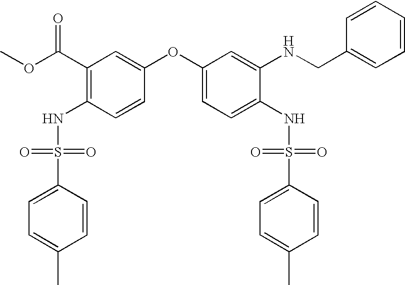

A solution of 5-(4-Amino-3-benzylamino-phenoxy)-2-(toluene-4-sulfonylamino)-benzoic acid methyl ester (141 mg, 0.272 mmol), tosyl chloride (63 mg, 0.33 mmol), and pyridine (43 mg, 0.54 mmol) in CH₂Cl₂ (2 mL) was stirred at 40° C. for 68 hours. The reaction was cooled to RT and diluted with CH₂Cl₂ (20 mL). The reaction mixture was washed with aq. 0.5 N KHSO₄ and aq. sat. NaHCO₃ (20 mL). The aqueous-layers were extracted once more with CH₂Cl₂ (20 mL). The combined CH₂Cl₂-layers were dried (Na₂SO₄), filtered, and evaporated to dryness in vacuo. The crude product was purified by column chromatography (EtOAc/heptane: 1/4 to 1/2) and then crystallized from EtOAc/heptane to afford the desired product as a white solid (93 mg, 51%, purity (LC)>95%).

MS: [M−H]⁻=670.

### Preparation of 5-[3-Benzylamino-4-(toluene-4-sulfonylamino)-phenoxy]-2-(toluene-4-sulfonylamino)-benzoic acid

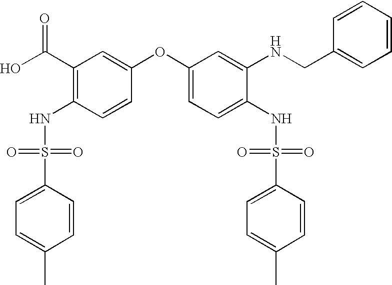

A mixture of 5-[3-Benzylamino-4-(toluene-4-sulfonylamino)-phenoxy]-2-(toluene-4-sulfonylamino)-benzoic acid methyl ester (93 mg, 0.138 mmol) in aq. 0.5 M LiOH (5 mL) and THF (5 mL) was stirred at 60° C. for 4 hours. The reaction was cooled to RT and THF was evaporated in vacuo. The residue was acidified with aq. 2 M HCl (4 mL) and then diluted with water (10 mL). The aqueous suspension was extracted with DCM (2×10 mL). The combined DCM-extracts were dried (Na₂SO₄), filtered, and evaporated to dryness in vacuo to obtain the crude product as a solid. The crude product was triturated with EtOAc/heptane: 1/1 to afford the desired product as a white solid (50 mg, 55%, purity (LC)>95%).

MS: [M−H]⁻=656.

NMR: ¹H (DMSO-d6): δ 9.22 (s, 1H), 7.65 (d, J=8.36 Hz, 2H), 7.57 (d, J=8.32 Hz, 2H), 7.45 (d, J=8.84 Hz, 1H), 7.37-7.32 (m, 4H), 7.23-7.14 (m, 4H), 7.09 (dd, J=2.76 Hz and J=8.84 Hz, 1H), 7.05-7.01 (m, 2H), 6.57 (d, J=8.56 Hz, 1H), 5.96-5.90 (m, 2H), 4.11-4.07 (m, 2H), 2.38 (s, 3H), 2.31 (s, 3H).

### Compound 9

### Preparation of N1-butyl-5-fluoro-2-nitroaniline

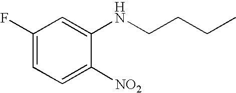

Under Argon and at RT, Pd(OAc)₂ (24 mg, 0.11 mmol) and BINAP (74 mg, 0.12 mmol) were added to a solution of butylamine (173 mg, 2.37 mmol) and 4-fluoro-2-bromonitrobenzene (460 mg, 2.09 mmol) in toluene (degassed by purging, 3 mL). The reaction mixture was heated at 100° C. for 3 minutes and then cooled to 0° C. Upon addition of sodium tert-butoxide (271 mg, 2.82 mmol) the reaction mixture turned red. The reaction was stirred at 70° C. for 16 hours. The reaction was cooled to RT and filtered over Kiezelguhr. The residue was rinsed with EtOAc (30 mL). The EtOAc-layer was washed with brine (30 mL). The aqueous-layer was extracted once more with EtOAc (30 mL). The combined EtOAc-extracts were dried (Na₂SO₄), filtered, and evaporated to dryness in vacuo. The crude product was purified by flash column chromatography (EtOAc/heptane: 1/100 to 1/30) to afford N1-butyl-5-fluoro-2-nitro-aniline as a yellow oil (333 mg, 75%, purity (GC)=85%).

MS: [M]+=212.

### Preparation of 5-(3-butylamino-4-nitro-phenoxy)-2-(toluene-4-sulfonylamino)-benzoic acid methyl ester

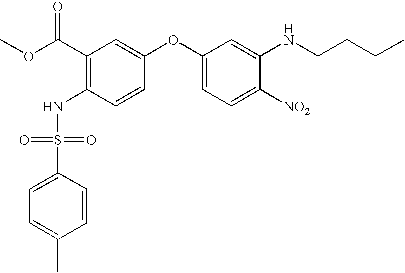

A mixture of methyl 5-hydroxy-2-[(4-methylphenyl)sulfonyl]aminobenzoate (514 mg mg, 1.60 mmol), N1-butyl-5-fluoro-2-nitroaniline (333 mg, 1.57 mmol), and potassium carbonate (466 mg, 3.37 mmol) in DMF (15 mL) was stirred at 80° C. for 7 hours. The reaction was cooled to RT, diluted with EtOAc (75 mL) and washed with aq. 0.5 N KHSO₄ (75 mL), aq. sat. NaHCO₃ (125 mL), and brine (125 mL). The aqueous-layers were extracted with EtOAc (75 mL). The combined EtOAc-extracts were dried (Na₂SO₄), filtered, and evaporated to dryness in vacuo. The crude product was purified by column chromatography (EtOAc/heptane: 1/9 to 1/4) to obtain 5-(3-butylamino-4-nitrophenoxy)-2-(toluene-4-sulfonylamino)-benzoic acid methyl ester as a yellow oil (590 mg, 73%, purity (LC)=80%).

MS: [M−H]⁻=512.

### Preparation of 5-(4-Amino-3-butylamino-phenoxy)-2-(toluene-4-sulfonylamino)-benzoic acid methyl ester

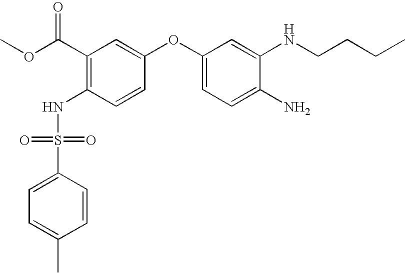

A suspension of 5-(3-butylamino-4-nitro-phenoxy)-2-(toluene-4-sulfonylamino)-benzoic acid methyl ester (591 mg, 1.15 mmol) and 10% Pd—C (86 mg) in THF/MeOH: 1/1 (10 mL) was placed under 1 atmosphere of hydrogen and stirred at RT for 17 hours. The reaction was filtered over Kiezelguhr and the residue was rinsed with THF. The filtrate was evaporated to dryness in vacuo. The crude product was purified by flash column chromatography (EtOAc/heptane: 1/4 to 1/2) to afford 5-(4-amino-3-butylamino-phenoxy)-2-(toluene-4-sulfonylamino)-benzoic acid methyl ester as a yellow oil (297 mg, 53%, purity (LC)>95%).

MS: [M−H]⁻=482

### Preparation of 5-[3-Butylamino-4-(toluene-4-sulfonylamino)-phenoxy]-2-(toluene-4-sulfonylamino)-benzoic acid methyl ester

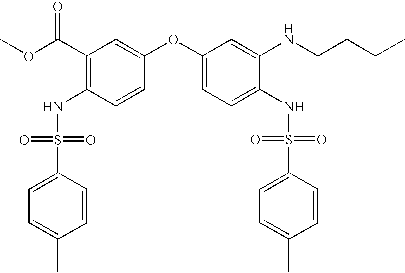

A solution of 5-(4-Amino-3-butylamino-phenoxy)-2-(toluene-4-sulfonylamino)-benzoic acid methyl ester (280 mg, 0.579 mmol), tosyl chloride (131 mg, 0.687 mmol), and pyridine (105 mg, 1.33 mmol) in CH₂Cl₂ (3 mL) was stirred at 40° C. for 23 hours. The reaction was cooled to RT and diluted with CH₂Cl₂ (20 mL). The reaction mixture was washed with aq. 0.5 N KHSO₄ (20 mL) and aq. sat. NaHCO₃ (20 mL). The aqueous-layers were extracted once more with CH₂Cl₂ (20 mL). The combined CH₂Cl₂-layers were dried (Na₂SO₄), filtered, and evaporated to dryness in vacuo. The crude product was purified by column chromatography (EtOAc/heptane: 1/2). According to LC, the product was still not pure. The product was purified by RP column chromatography (CH₃CN/H₂O: 2/1) to obtain the desired product as a colourless oil (140 mg, 38%, purity (LC)>95%).

MS: [M+H]⁺=638.

NMR: ¹H (CDCl₃): δ 10.30 (s, 1H), 7.72-7.62 (m, 5H), 7.51 (d, J=3.00 Hz, 1H), 7.30-7.20 (m, 4H), 7.12 (dd, J=2.80 Hz and J=9.08 Hz, 1H), 6.34 (d, J=8.60 Hz, 1H), 6.18 (d, J=2.52 Hz, 1H), 5.87 (dd, J=2.52 Hz and J=8.60 Hz, 1H), 5.73 (s, 1H), 4.64 (bs, 1H), 3.82 (s, 3H), 2.94 (q, J=6.80 Hz, 2H), 2.43 (s, 3H), 2.38 (s, 3H), 1.55-1.47 (m, 2H), 1.42-1.31 (m, 2H), 0.92 (t, J=7.32 Hz, 3H).

### Preparation of 5-[3-Butylamino-4-(toluene-4-sulfonylamino)-phenoxy]-2-(toluene-4-sulfonylamino)-benzoic acid

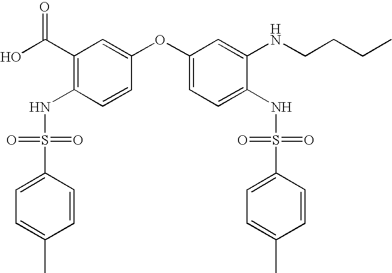

A solution of 5-[3-Butylamino-4-(toluene-4-sulfonylamino)-phenoxy]-2-(toluene-4-sulfonylamino)-benzoic acid methyl ester (125 mg, 0.196 mmol) in aq. 0.5 M LiOH (6 mL) and THF (6 mL) was stirred at 60° C. for 3 hours. The reaction was cooled to RT and THF was evaporated in vacuo. The residue was acidified with aq. 1 M HCl (10 mL). The aqueous suspension was extracted with DCM (2×20 mL). The combined DCM-extracts were dried (Na₂SO₄), filtered, and evaporated to dryness in vacuo. According to LC, the crude product was 94+% purity. The crude product was dissolved in aq. 0.5 M LiOH (1 mL) and then aq. 1 M HCl (4 mL) was added. The precipitate was filtered, rinsed with water and dried at 40° C. in vacuo for 16 hours to afford the desired product as a solid (100 mg, 82%, purity (LC)>95%).

MS: [M−H]⁻=652.

NMR: ¹H (DMSO-d6): δ 10.72 (s, 1H), 9.18 (s, 1H), 7.65 (d, J=8.36 Hz, 2H), 7.54-7.48 (m, 3H), 7.37-7.30 (m, 5H), 7.24 (dd, J=3.04 Hz and J=8.84 Hz, 1H), 6.65 (d, J=8.32 Hz, 1H), 6.08 (d, J=2.56 Hz, 1H), 5.97 (dd, J=2.56 Hz and J=8.32 Hz, 1H), 2.74 (t, J=6.60 Hz, 2H), 2.35 (s, 3H), 2.34 (s, 3H), 1.32-1.14 (m, 4H), 0.82 (t, J=7.08 Hz, 3H).

### Compound 10

### Preparation of 5-[3-(Benzyl-methyl-amino)-4-(toluene-4-sulfonylamino)-phenoxy]-2-(toluene-4-sulfonylamino)-benzoic acid methyl ester

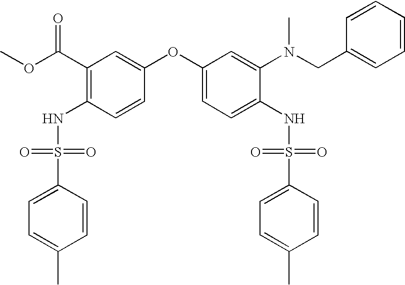

A solution of 5-[4-Amino-3-(benzyl-methyl-amino)-phenoxy]-2-(toluene-4-sulfonyl-amino)-benzoic acid methyl ester (225 mg, 0.42 mmol), p-toluenesulfonylchloride (89 mg, 0.46 mmol) and pyridine (66 mg, 0.83 mmol) in dichloromethane (5 mL) was stirred for 20 hours at 40° C. The reaction mixture was evaporated until dryness, 1,2-dichlororethane (5 mL) was added and the reaction was heated to 60° C. for 16 hours. The reaction was diluted with dichloromethane (50 mL) and washed with aqueous 0.5N potassium hydrogen sulfate (30 mL), saturated sodium hydrogen carbonate (30 mL) and dried over anhydrous sodium sulfate. After concentration under reduced pressure the residue was purified by column chromatography (SiO₂) on gradient elution with heptane/EtOAc from 100:0 to 80:20 to obtain the desired product (155 mg, 49%, purity (LC)>95%).

MS: [M+H]⁺=686

### Preparation of 5-[3-(Benzyl-methyl-amino)-4-(toluene-4-sulfonylamino)-phenoxy]-2-(toluene-4-sulfonylamino)-benzoic acid

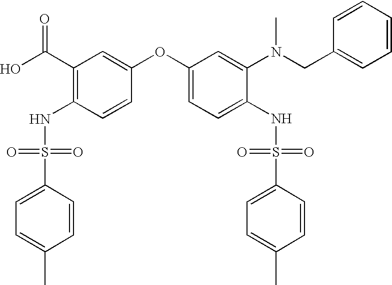

A mixture of 5-[3-(Benzyl-methyl-amino)-4-(toluene-4-sulfonylamino)-phenoxy]-2-(toluene-4-sulfonylamino)-benzoic acid methyl ester (155 mg, 0.23 mmol), LiOH (39 mg, 0.94 mmol), water (2 mL) and THF (2 mL) was stirred for 16 hours at 60° C. The reaction was acidified to pH=1 with aq. 1N HCl and the THF was evaporated under reduced pressure. The suspension was extracted with DCM (2×50 mL) and the extracts were dried over anhydrous sodium sulfate. Concentration of the organic solution afforded the crude product, which was purified with preperative TLC (Merck, 12 PLC-plates 20×20 cm silica gel 60 F₂₅₄, 2 mm with concentrating zone 20×4 cm), eluens: dichloromethane (95)/2% acetic acid in methanol (5) and reversed phase flah-column chromatografie (Si—C18) on elution with 0.35% ammonia/acetonitrile from 100:0 to 60:40 to obtain the desired product (80 mg, 51%, purity (LC)>95%).

MS: [M−H]⁻=609

NMR: ¹H (DMSO-d6): δ 10.85 (bs, 1H), 9.10 (s, 1H), 7.68 (d, J=8.32 Hz, 2H), 7.65 (d, J=8.32 Hz, 2H), 7.46 (d, J=9.88 Hz, 1H), 7.34 (dd, J=2.04 Hz and 8.36 Hz, 4H), 7.26 (d, J=3.04 Hz, 1H), 7.23-7.18 (m, 3H), 7.15-7.11 (m, 2H), 7.07 (dd, J=2.76 Hz and 8.84 Hz, 1H), 7.01 (d, J=8.56 Hz, 1H), 6.56 (d, J=2.52, 1H), 6.52 (dd, J=2.52 Hz and 8.60 Hz, 1H), 3.93 (s, 2H), 2.39 (s, 3H), 2.34 (s, 3H), 2.32 (s, 3H)

### Compound 11

### Preparation of N1-ethyl-5-fluoro-2-nitroaniline

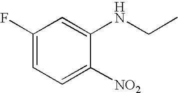

Under Argon and at RT, Pd(OAc)₂ (33 mg, 0.15 mmol) and BINAP (101 mg, 0.16 mmol) were added to a solution of 2 M ethylamine/THF (3.8 mL, 7.6 mmol) and 4-fluoro-2-bromonitrobenzene (647 mg, 2.94 mmol) in toluene (degassed by purging, 3 mL). The reaction mixture was heated at 90° C. for 3 minutes and then cooled to 0° C. Upon addition of sodium tert-butoxide (452 mg, 4.70 mmol) the reaction mixture turned red. The reaction was stirred at 70° C. for 22 hours. The reaction was filtered over Kiezelguhr. Rinsed with EtOAc (100 mL). The filtrate was washed with brine (100 mL). The brine-layer was extracted once with EtOAc (100 mL). The combined EtOAc-extracts were dried (Na₂SO₄), filtered, and evaporated to dryness in vacuo. The crude product was purified by column chromatography (heptane to EtOAc/heptane: 1/20) to obtain the title compound as a yellow solid (382 mg, 71%, purity (GC)>95%).

MS: [M]+=184.

### Preparation of 5-(3-ethylamino-4-nitro-phenoxy)-2-(toluene-4-sulfonylamino)-benzoic acid methyl ester

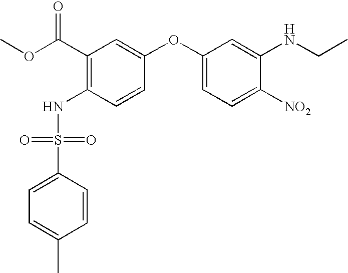

A mixture of methyl 5-hydroxy-2-[(4-methylphenyl)sulfonyl]aminobenzoate (413 mg, 1.29 mmol), N1-ethyl-5-fluoro-2-nitroaniline (257 mg, 1.40 mmol), and potassium carbonate (358 mg, 2.59 mmol) in DMF (10 mL) was stirred at 80° C. for 8 hours. The reaction was cooled to RT, diluted with EtOAc (100 mL) and washed with aq. 0.5 N KHSO₄ (125 mL), aq. sat. NaHCO₃ (125 mL), and brine (100 mL). The aqueous-layers were extracted with EtOAc (100 mL). The combined EtOAc-extracts were dried (Na₂SO₄), filtered, and evaporated to dryness in vacuo. The crude product was purified by column chromatography (EtOAc/heptane: 1/4) to obtain desired product as a yellow foam (527 mg, 84%, purity (LC)>95%).

MS: [M−H]⁻=484.

### Preparation of 5-(4-Amino-3-ethylamino-phenoxy)-2-(toluene-4-sulfonylamino)-benzoic acid methyl ester

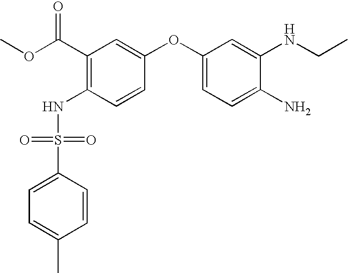

A suspension of 5-(3-ethylamino-4-nitro-phenoxy)-2-(toluene-4-sulfonylamino)-benzoic acid methyl ester (527 mg, 1.09 mmol) and 10% Pd—C (70 mg) in THF/MeOH: 1/1 (10 mL) was placed under 1 atmosphere of hydrogen and stirred at RT for 18 hours. The reaction was filtered over Kiezelguhr. Residue was rinsed with THF (20 mL). The filtrate was evaporated to dryness to afford the desired product (463 mg, 93%, purity (LC)>95%).

MS: [M−H]⁻=454.

NMR: ¹H (CDCl₃): δ 10.17 (s, 1H), 7.67 (d, J=8.08 Hz, 2H), 7.63 (d, J=9.12 Hz, 1H), 7.45 (d, J=3.04 Hz, 1H), 7.21 (d, J=8.08 Hz, 2H), 7.10 (dd, J=3.00 Hz and 9.08 Hz, 1H), 6.65 (d, J=8.08 Hz, 1H), 6.29 (d, J=2.56 Hz, 1H), 6.21 (dd, J=2.56 Hz and J=8.08 Hz), 3.78 (s, 3H), 3.06 (q, J=7.08 Hz, 2H), 2.37 (s, 3H), 1.28 (t, J=7.08 Hz, 3H).

### Preparation of 5-[3-Ethylamino-4-(toluene-4-sulfonylamino)-phenoxy]-2-(toluene-4-sulfonylamino)-benzoic acid methyl ester

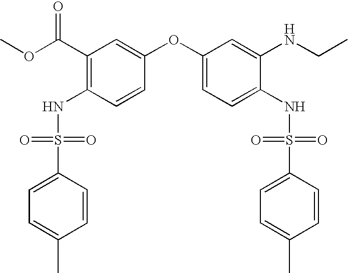

A solution of 5-(4-amino-3-ethylamino-phenoxy)-2-(toluene-4-sulfonylamino)-benzoic acid methyl ester (448 mg, 0.983 mmol), tosyl chloride (174 mg, 0.913 mmol), and pyridine (165 mg, 2.09 mmol) in CH₂Cl₂ was stirred at 40° C. for 16 hours. The reaction was cooled to RT and diluted with CH₂Cl₂ (50 mL). The reaction mixture was washed with aq. 0.5 N KHSO₄ (50 mL) and aq. sat. NaHCO₃ (50 mL). The aqueous-layers were extracted once more with CH₂Cl₂ (50 mL). The combined CH₂Cl₂-layers were dried (Na₂SO₄), filtered, and evaporated to dryness in vacuo. The crude product was purified by column chromatography (EtOAc/heptane: 1/3 to 1/2) and then triturated with heptane (5 mL) to obtain the desired product (418 mg, 70%, purity (LC)>95%).

MS: [M−H]⁻=608.

NMR: ¹H (DMSO-d6): δ 10.03 (bs, 1H), 9.19 (bs, 1H), 7.60 (d, J=8.12 Hz, 2H), 7.54 (d, J=8.32 Hz, 1H), 7.42 (d, J=8.84 Hz, 1H), 7.37-7.32 (m, 4H), 7.26 (d, J=2.76 Hz, 2H), 7.23 (d, J=2.76 Hz, 1H), 7.21 (d, J=3.04 Hz, 1H), 6.64 (d, J=8.60 Hz, 1H), 6.10 (d, J=2.76, 1H), 5.97 (dd, J=2.76 Hz and 8.60 Hz, 1H), 3.73 (s, 3H), 2.79 (q, J=7.08 Hz, 2H), 2.36 (s, 3H), 2.34 (s, 3H), 0.96 (t, J=7.08 Hz, 3H).

### Preparation of 5-[3-Ethylamino-4-(toluene-4-sulfonylamino)-phenoxy]-2-(toluene-4-sulfonylamino)-benzoic acid

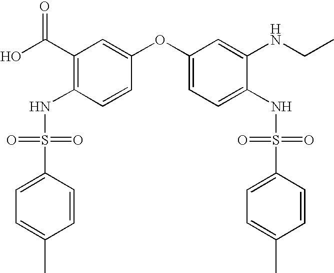

A solution of 5-[3-Ethylamino-4-(toluene-4-sulfonylamino)-phenoxy]-2-(toluene-4-sulfonylamino)-benzoic acid methyl ester (346 mg, 0.567 mmol) in 0.5 M LiOH (7 mL) and THF (7 mL) was stirred at 60° C. for 4 hours. The reaction was cooled to RT and evaporated in vacuo to remove THF. The residue was acidified with aq. 1 M HCl (7 mL). The precipitate was filtered and rinsed with water (10 mL). The precipitate was dried in vacuo for 16 hours. The crude product was purified by flash column chromatography (CH₂Cl₂/MeOH/AcOH: 90/10/1). The product was collected, evaporated in vacuo, and stripped with toluene (2×) and CH₂Cl₂ (2×) to afford the desired product as a light pink solid (314 mg, 93%, purity (LC)>95%).

MS: [M−H]⁻=594.

NMR: ¹H (DMSO-d6): δ 10.79 (bs, 1H), 9.16 (bs, 1H), 7.64 (d, J=8.08 Hz, 2H), 7.54-7.49 (m, 3H), 7.38-7.30 (m, 5H), 7.28-7.12 (m, 3H), 6.63 (d, J=8.60 Hz, 1H), 6.08 (d, J=2.80 Hz, 1H), 5.97 (dd, J=2.80 Hz and 8.60 Hz, 1H), 4.87 (bs, 1H), 2.78 (q, J=7.06, 2H), 2.35 (s, 3H), 2.33 (s, 3H), 0.94 (t, J=7.06, 3H).

### Compound 12

### Preparation of 2-bromo-4-fluoronitrobenzene

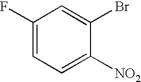

A 500 mL RB-flask was charged with commercially available 1-bromo-3-fluoro-benzene (30.0 g, 0.171 mol) and methane sulfonic acid (100 mL). The reaction was vigorously stirred and sodium nitrate (14.6 g, 0.171 mmol) was added in small portions to the reaction mixture while the temperature was maintained below 30° C. using a water bath for external cooling. After addition of sodium nitrate, the reaction was stirred at RT for 5 hours. The reaction mixture was poured into 500 mL ice-water and the aqueous layer was extracted with dichloromethane (2×250 mL). The organic extracts were washed once with saturated NaHCO₃/H₂O (250 mL). The combined extracts were dried (Na₂SO₄), filtered, and evaporated to dryness in vacuo. The crude product was distilled (bp 85° C., 2.3 torr) to afford a mixture of 2-bromo-4-fluoronitrobenzene and 2-fluoro-4-bromonitrobenzene (ratio 5/1) as a light yellow oil, which solidified upon standing at room temperature (30.5 g, 81%), in a yield of 30.5 g (81%). 2-bromo-4-fluoronitrobenzene could be obtained pure by recrystallization from a little warm methanol.

### Preparation of 2-benzyl-4-fluoro-nitrobenzene

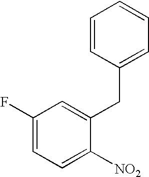

At RT and under Argon, PdCl₂(dppf) (118 mg, 0.16 mmol) was added to a suspension of 2-bromo-4-fluoronitrobenzene (355 mg, 1.61 mmol), potassium benzyltrifluoro-borate (353 mg, 1.78 mmol), and cesium carbonate (1.54 g, 4.73 mmol) in THF/water: 5/1 (degassed by purging, 6 mL). The reaction was warmed at reflux for 22 hours. An additional amount of potassium benzyltrifluoroborate (159 mg), PdCl₂(dppf) (57 mg), THF (4 mL), and water (1 mL) were added and the reaction was continued for 24 hours. The reaction was filtered over Kiezelguhr and the residue was rinsed with THF (20 mL). THF was removed in vacuo. The residue was dissolved in EtOAc (30 mL) and was washed with brine (2×30 mL). The aqueous-layer was extracted once more with EtOAc (20 mL). The EtOAc-extracts were dried (Na₂SO₄), filtered, and evaporated to afford the crude product as dark brown oil. The crude product was purified by flash column chromatography (EtOAc/heptane: 1/50) to obtain the desired product as a light yellow oil (257 mg, 69%, purity (LC) n.d).

NMR: ¹H (CDCl₃): δ 8.03 (dd, J=5.04 Hz and 8.84 Hz, 1H), 7.35-7.23 (m, 3H), 7.14-7.19 (m, 2H), 7.05 (ddd, J=2.78 Hz and 7.07 Hz and 8.84 Hz, 1H), 6.91 (dd, J=2.78 Hz and 9.09 Hz, 1H), 4.33 (s, 2H).

### Preparation of 5-(3-benzyl-4-nitro-phenoxy)-2-(toluene-4-sulfonylamino)-benzoic acid methyl ester

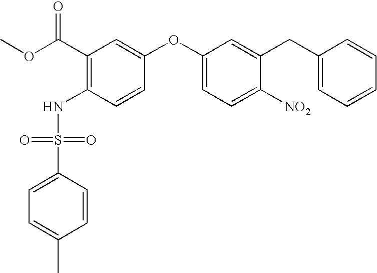

A mixture of methyl 5-hydroxy-2-[(4-methylphenyl)sulfonyl]aminobenzoate (346 mg, 1.07 mmol), 2-benzyl-4-fluoro-nitrobenzene (248 mg, 1.07 mmol), and potassium carbonate (295 mg, 2.14 mmol) in DMF (10 mL) was stirred at 80° C. for 5 hours. The reaction was cooled to RT, diluted with EtOAc (70 mL) and washed with aq. 0.5 N KHSO₄ (70 mL), aq. sat. NaHCO₃ (70 mL), and brine (70 mL). The aqueous-layers were extracted with EtOAc (50 mL). The combined EtOAc-extracts were dried (Na₂SO₄), filtered, and evaporated to dryness in vacuo. The crude product was purified by column chromatography (EtOAc/heptane: 1/4) to obtain the desired product as a yellow oil (383 mg, 67%, purity (LC)=90%)

MS: [M−H]⁻=531.

### Preparation of 5-(4-Amino-3-benzyl-phenoxy)-2-(toluene-4-sulfonylamino)-benzoic acid methyl ester

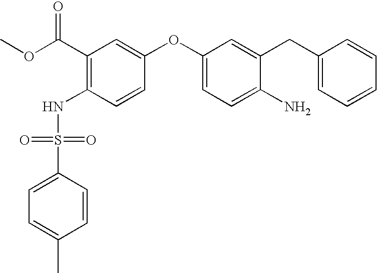

A suspension of 5-(3-benzyl-4-nitro-phenoxy)-2-(toluene-4-sulfonylamino)-benzoic acid methyl ester (380 mg, 0.714 mmol) and 10% Pd—C (38 mg) in THF/MeOH: 1/1 (10 mL) was placed under 1 atmosphere of hydrogen and stirred at RT for 72 hours. The reaction was filtered over Kiezelguhr. Rinsed with THF (30 mL). The filtrate was evaporated to dryness in vacuo. The crude product was purified by column chromatography (EtOAc/heptane: 1/4 to 1/2) to afford the desired product as a white foam (yield was not determined, purity (LC)>95%).

MS: [M−H]⁻=501.

### Preparation of 5-[3-Benzyl-4-(toluene-4-sulfonylamino)-phenoxy]-2-(toluene-4-sulfonylamino)-benzoic acid methyl ester

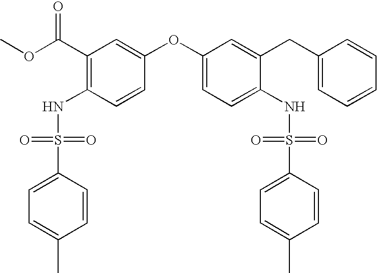

A solution of 5-(4-Amino-3-benzyl-phenoxy)-2-(toluene-4-sulfonylamino)-benzoic acid methyl ester (200 mg, 0.398 mmol), tosyl chloride (128 mg, 0.671 mmol), and pyridine (67 mg, 0.85 mmol) in CH₂Cl₂ (5 mL) was stirred at reflux for 21 hours. The reaction was cooled to RT, diluted with CH₂CL₂ (25 mL) and washed with aq. 0.5 N KHSO₄ (2×25 mL). The aqueous-layers were extracted once more with CH₂Cl₂ (25 mL). The combined CH₂Cl₂-extracts were dried (Na₂SO₄), filtered, and evaporated to dryness in vacuo. The crude product was purified by flash column chromatography (EtOAc/heptane: 1/4 to 1/2) to obtain the desired product solid (230 mg, 88%, purity (LC)>95%).

MS: [M−H]⁻=655.

NMR: ¹H (CDCl₃): δ 10.30 (bs, 1H), 7.72-7.66 (m, 3H), 7.54 (d, J=8.36 Hz, 2H), 7.51 (d, J=2.76 Hz, 1H), 7.30-7.20 (m, 9H), 7.11 (dd, J=2.76 Hz and J=9.08 Hz, 1H), 6.95-6.91 (m, 2H), 6.73-6.67 (m, 2H), 3.83 (s, 3H), 3.53 (s, 2H), 2.43 (s, 3H), 2.37 (s, 3H).

### Preparation of 5-[3-Benzyl-4-(toluene-4-sulfonylamino)-phenoxy]-2-(toluene-4-sulfonylamino)-benzoic acid

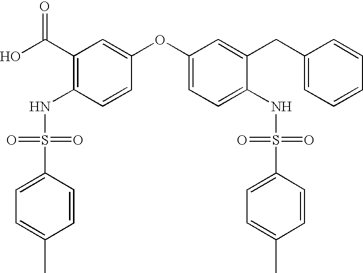

A solution of methyl 5-[3-Benzyl-4-(toluene-4-sulfonylamino)-phenoxy]-2-(toluene-4-sulfonylamino)-benzoate in 0.5 M aq. lithium hydroxide (5 mL) and tetrahydrofuran (5 mL) was stirred at 60° C. for 3 hours. The reaction was cooled to RT and then the reaction was evaporated in vacuo to remove THF. The residue was acidified with aq. 1 M HCl (6 mL). The precipitate was filtered and rinsed with water (20 mL). The residue was dried in vacuo for 16 hours to afford the desired product as a light pink solid (180 mg, 93%, purity (LC)>95%).

MS: [M−H]⁻=641.

NMR: ¹H (DMSO-d6): δ 11.25 (bs, 1H), 9.59 (s, 1H), 7.64 (d, J=8.08 Hz, 2H), 7.54 (d, J=8.08 Hz, 2H), 7.47 (d, J=9.08 Hz, 1H), 7.38-7.32 (m, 4H), 7.27 (d, 3.04 Hz, 1H), 7.24-7.12 (m, 4H), 6.93 (d, J=6.84, 2H), 6.81 (d, J=8.84 Hz, 1H), 6.67 (dd, J=2.76 Hz and 8.84 Hz, 1H), 6.52 (d, J=2.76 Hz, 1H), 3.80 (s, 2H), 2.38 (s, 3H), 2.33 (s, 3H).

### Compound 13

### Preparation of Benzyl-(5-fluoro-2-nitro-phenyl)-methyl-amine

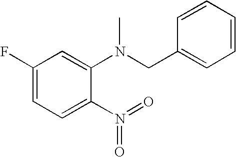

Under Argon and at RT, Pd(OAc)₂ (43 mg, 0.19 mmol) and BINAP (124 mg, 0.20 mmol) were added to a solution of benzylmethylamine (340 mg, 2.81 mmol) and 4-fluoro-2-bromonitrobenzene (624 mg, 2.81 mmol) in toluene (degassed by purging, 5 mL). The reaction mixture was heated at 100° C. for 3 minutes and then cooled to 0° C. Upon addition of sodium tert-butoxide (324 mg, 3.37 mmol) the reaction mixture turned red. The reaction was stirred at 70° C. for 20 hours. The reaction was cooled to RT and filtered over Kiezelguhr. The residue was rinsed with EtOAc (50 mL). The EtOAc-layer was washed with brine (50 mL). The EtOAc-extract was dried (Na₂SO₄), filtered, and evaporated to dryness in vacuo. The crude product was purified by flash column chromatography (EtOAc/heptane: 1/16) to afford Benzyl-(5-fluoro-2-nitro-phenyl)-methylamine as a yellow oil (443 mg, 61%, purity (LC) n.d.).

NMR: ¹H (CDCl3): δ 7.85 (dd, J=6.32 Hz and J=9.12 Hz, 1H), 7.38-7.21 (m, 5H), 6.69 (dd, J=2.52 Hz, 1H), 6.56 (ddd, J=2.52 Hz and J=7.08 Hz and J=9.08 Hz, 1H), 4.40 (s, 2H), 2.80 (s, 3H).

### Preparation of 5-[3-(benzyl-methyl-amino)-4-nitro-phenoxy]-2-(toluene-4-sulfonyl-amino)-benzoic acid methyl ester

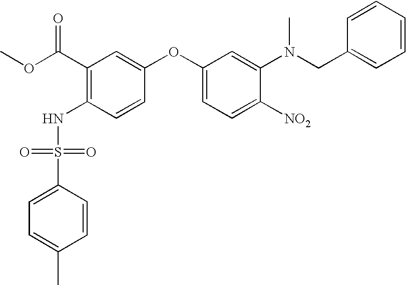

A mixture of methyl 5-hydroxy-2-[(4-methylphenyl)sulfonyl]aminobenzoate (536 mg, 1.67 mmol), N1-benzyl-N1-methyl-5-fluoro-2-nitroaniline (434 mg, 1.67 mmol), and potassium carbonate (453 mg, 3.28 mmol) in DMF (10 mL) was stirred at 80° C. for 6 hours. The reaction was diluted with EtOAc (75 mL) and washed with aq. 0.5 M KHSO₄ (50 mL), aq. sat. NaHCO₃ (75 mL), and brine (50 mL). The aqueous-layers were extracted with EtOAc (75 mL). The combined EtOAc-extracts were dried (Na₂SO₄), filtered, and evaporated to dryness in vacuo. The crude product was purified by column chromatography (EtOAc/heptane: 1/10 to 1/4) to obtain the desired product as a yellow oil (490 mg, 52%, purity (LC)=70%).

MS: [M+H]⁺=562.

5-[4-Amino-3-(benzyl-methyl-amino)-phenoxy]-2-(toluene-4-sulfonylamino)-benzoic acid methyl ester

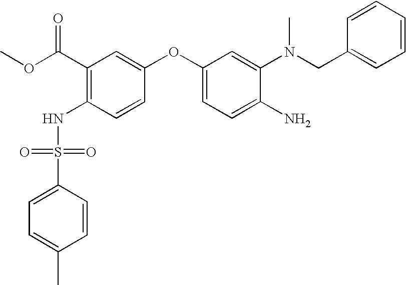

At RT and under nitrogen, a solution of 5-[3-(benzyl-methyl-amino)-4-nitro-phenoxy]-2-(toluene-4-sulfonylamino)-benzoic acid methyl ester (563 mg, 1.00 mmol) in THF/MeOH: 1/1 (6 mL) was added to a suspension of iron (280 mg, 5.01 mmol) and ammonium chloride (290 mg, 5.42 mmol) in water (6 mL). The reaction was heated to 70° C. for 3 hours and then an additional amount of iron (150 mg) and ammonium chloride (209 mg) were added and the reaction was continued for 3 hours. The reaction was cooled to RT and filtered over Kiezelguhr. Rinse with CH₂Cl₂ (50 mL). The filtrate was washed with aq. sat. NaHCO₃ (1×30 mL). The aqueous-layer was extracted once more with CH₂Cl₂ (20 mL). The combined CH₂CL₂-extracts were dried (Na₂SO₄), filtered and evaporated to dryness in vacuo. The residue was purified by flash column chromatography (EtOAc/heptane: 1/4 to 1/2) to afford the desired product as a white foam (335 mg, 63%, purity (LC)=93%).

MS: [M+H]⁺=532

### Preparation of methyl 5-(3-[benzyl(methyl)amino]-4-[(4-methylphenyl)sulfonyl]amino-phenoxy)-2-[(4-methylphenyl)sulfonyl]aminobenzoate

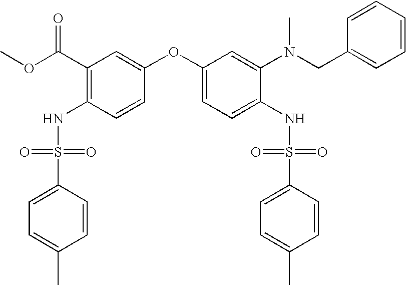

A solution of 5-[4-Amino-3-(benzyl-methyl-amino)-phenoxy]-2-(toluene-4-sulfonyl-amino)-benzoic acid methyl ester (335 mg, 0.630 mmol), tosyl chloride (180 mg, 0.944 mmol), and pyridine (110 mg, 1.39 mmol) in dichloromethane (5 mL) was stirred at reflux for 24 hours. The reaction mixture was diluted with CH₂CL₂ (20 mL) and washed with aq. 0.5 N KHSO₄ (30 mL), aq. sat. NaHCO₃ (30 mL). The aqueous-layers were extracted once more with CH₂Cl₂ (20 mL). The combined CH₂Cl₂-extracts were dried (Na₂SO₄), filtered, and evaporated to dryness in vacuo. The product was purified by flash column chromatography (EtOAc/heptane: 1/4 to 1/2) and then by RP flash column chromatography (CH₃CN/H₂O: 4/1) to obtain the desired product (200 mg, 46%, purity (LC)>95%).

MS: [M−H]⁻=684.

### Preparation of 5-[3-Methylamino-4-(toluene-4-sulfonylamino)-phenoxy]-2-(toluene-4-sulfonylamino)-benzoic acid methyl ester

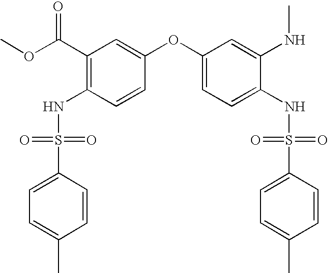

10% Pd—C (47 mg) was added to a solution of methyl 5-(3-[benzyl(methyl)amino]-4-[(4-methylphenyl)sulfonyl]aminophenoxy)-2-[(4-methylphenyl)sulfonyl]amino-benzoate (200 mg, 0.292 μmol) in THF/EtOH/AcOH: 1/1/1 (6 mL). The reaction was placed under 1 atmosphere of hydrogen and was stirred at 50° C. for 6 hours. The reaction mixture was filtered over Kiezelguhr. Rinse with EtOAc (30 mL). The filtrate was evaporated to dryness in vacuo. Ethylacetate (25 mL) and added and the organic layer was washed with aq. sat. NaHCO₃ (30 mL) and brine (30 mL). The aqueous-layers were extracted once more with EtOAc (25 mL). The combined EtOAc-extracts were dried (Na₂SO₄), filtered, and evaporated to dryness in vacuo. The product was purified by flash column chromatography (EtOAc/heptane: 1/2) to afford the desired product as a white solid (118 mg, 68%, purity (LC)>95%).

MS: [M+H]⁺=596, [M−H]⁻=594.

### Preparation of 5-[3-Methylamino-4-(toluene-4-sulfonylamino)-phenoxy]-2-(toluene-4-sulfonylamino)-benzoic acid

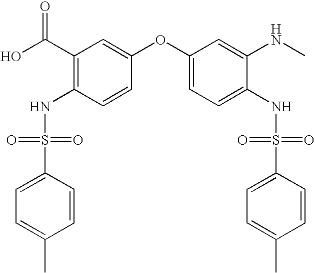

A solution of 5-[3-Methylamino-4-(toluene-4-sulfonylamino)-phenoxy]-2-(toluene-4-sulfonylamino)-benzoic acid methyl ester (118 mg, 198 μmol) in 0.5 M aq. lithium hydroxide (5 mL) and tetrahydrofuran (5 mL) was stirred at 60° C. After 2 hours, the reaction was cooled to RT. The reaction was evaporated in vacuum to remove tetrahydrofuran. The residue was acidified with aq. 1 M hydrochloric acid (6 mL). The precipitate was filtered and rinsed with water (20 mL). The residue was dried in vacuo for 16 hours to afford the desired product as a light pink solid (110 mg, 95%, purity (LC)>95%).

MS: [M−H]⁻=580.

NMR:¹H (DMSO-d6): δ 10.72 (s, 1H), 9.12 (s, 1H), 7.64 (d, J=8.36 Hz, 2H), 7.55 (d, J=8.08 Hz, 2H), 7.51 (d, J=8.84 Hz, 1H), 7.38-7.32 (m, 4H), 7.31 (d, J=3.00 Hz, 1H), 7.23 (dd, J=3.00 Hz and J=8.84 Hz, 1H), 6.44 (d, J=8.60 Hz, 1H), 6.07 (d, J=2.56 Hz, 1H), 5.91 (dd, J=2.56 Hz and 8.36 Hz, 1H), 2.36 (s, 3H), 2.33 (s, 3H)

### Preparation of Compounds (14-49) and Tested in the HIV-HTRF Test System According to the Invention

### Compound 14

### Preparation of 3,3′-methylenebis[6-(amino)-benzoic acid dimethyl ester]

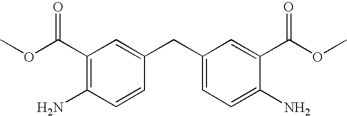

Methyl anthranilate (40 g, 265 mmol) was dissolved in methanol (250 ml). Formaldehyde 37% solution in water (9.9 ml, 132.5 mmol) and HCl 37% solution in water (32 ml, 383 mmol) were added. The solution was refluxed for 16 hours. Isopropyl ether was added and the crystals were filtered off The crude product was used in the next step without further purification (45 g, 90%).

MS: [M+H]⁺=315

### Preparation of 3,3′-methylenebis[6-(4-methyl phenyl sulfonamino)-benzoic acid dimethyl ester]

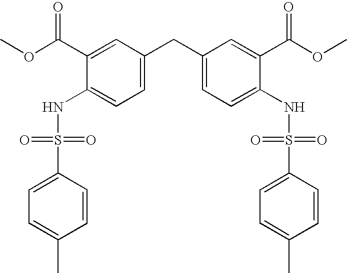

3,3′-methylenebis[6-(amino)-benzoic acid dimethyl ester] (10 g, 32 mmol) and p-toluenesulfonyl chloride were dissolved in pyridine (200 ml). The solution was stirred at room temperature for 64 hours. The solvent was evaporated under reduced pressure. The residue was purified by column chromatography (SiO₂) on elution with dichloromethane (18 g, 90%).

MS: [M+NH₄⁺]=640

### Preparation of 5-[3-Methoxycarbonyl-4-(4-methyl phenyl sulfonamino)-benzyl]-2-(4-methyl phenyl sulfonamino)-benzoic acid

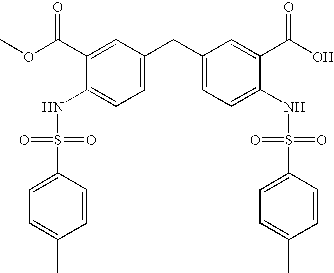

3,3′-methylenebis[6-(4-methyl phenyl sulfonamino)-benzoic acid dimethyl ester] (2.5 g, 4 mmol) was dissolved in tetrahydrofurane (25 ml). Lithium hydroxide monohydrate (48 mg, 2 mmol) dissolved in water (25 ml) was added. The solution was stirred at room temperature for 64 hours. The solvents were evaporated under reduced pressure. The residue was extracted with ethyl acetate/water, acidified with HCl until pH 7. The organic layer was dried over magnesium sulfate and evaporated under reduced pressure. The residue was purified by column chromatography (SiO₂) on elution with dichloromethane (2.4 g, 13.5%).

MS: [M−H]⁻=607

### Preparation of 5-[3-Hydroxymethyl-4-(4-methyl phenyl sulfonamino)-benzyl]-2-(4-methyl phenyl sulfonamino)-benzoic acid methyl ester

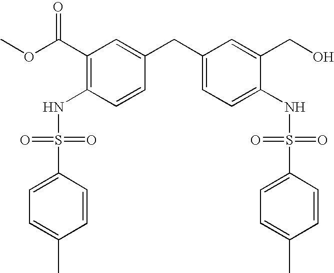

5-[3-Benzoic acid-4-(4-methyl phenyl sulfonamino)-benzyl]-2-(4-methyl phenyl sulfonamino)-benzoic acid methyl ester (325 mg, 534 μmol) was dissolved in tetrahydrofurane (20 ml, extra dry). The solution was flushed with nitrogen, sealed and cooled to 0° C. Borane-tetrahydrofurane complex (1.6 ml, 16.5 mmol) was added slowly. The solution was stirred at room temperature for 16 hours. The solution was slowly diluted with water and extracted with dichloromethane. The organic layer was dried over magnesium sulfate and evaporated under reduced pressure. The residue was purified by column chromatography (SiO₂) on elution with ethyl acetate:heptane 4:6 (83 mg, 24%).

MS: [M−H]⁻=593

### Preparation of 5-[3-Hydroxymethyl-4-(4-methyl phenyl sulfonamino)-benzyl]-2-(4-methyl phenyl sulfonamino)-benzoic acid

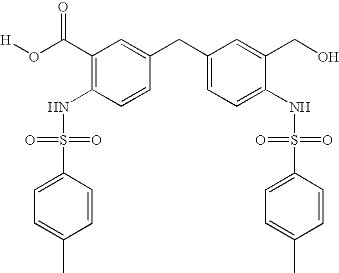

5-[3-Hydroxymethyl-4-(4-methyl phenyl sulfonamino)-benzyl]-2-(4-methyl phenyl sulfonamino)-benzoic acid methyl ester (83 mg, 140 μmol) was dissolved in tetrahydrofurane (20 ml). Lithium hydroxide monohydrate (117 mg, 279 μmol) dissolved in water (20 ml) was added. The solution was stirred at room temperature for 16 hours. The solvents were evaporated under reduced pressure. The residue was extracted with dichloromethane/water, acidified with HCl until pH 7. The organic layer was dried over magnesium sulfate and evaporated under reduced pressure. The crude product was purified by preparative HPLC (Waters Xterra Prep MC C18 (5 μm, 19×50 mm), eluens: H₂O (A), acetonitrile (B), 0.1M ammonium formate+4% formic acid in H₂O (C) from 55A/40B/5C to 0A/95B/5C in 10 minutes) (28.4 mg, 33%, purity (LC)=94.39%).

MS: [M−H]⁻=579

### Compound 15

### Preparation of 3,3′-methylenebis[6-(amino)-benzoic acid dimethyl ester]

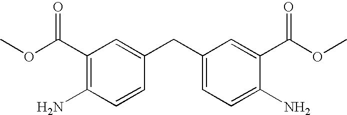

Methyl anthranilate (40 g, 265 mmol) was dissolved in methanol (250 ml). Formaldehyde 37% solution in water (9.9 ml, 132.5 mmol) and HCl 37% solution in water (32 ml, 383 mmol) were added. The solution was refluxed for 16 hours.

Isopropyl ether was added and the crystals were filtered off The crude product was used in the next step without further purification (45 g, 90%).

MS: [M+H]⁺=315

### Preparation of 3,3′methylenebis[6-(4-methyl phenyl sulfonamino)-benzoic acid dimethyl ester]

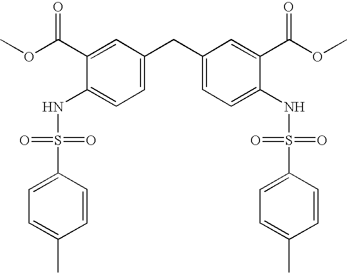

3,3′-methylenebis[6-(amino)-benzoic acid dimethyl ester] (10 g, 32 mmol) and p-toluenesulfonyl chloride were dissolved in pyridine (200 ml). The solution was stirred at room temperature for 64 hours. The solvent was evaporated under reduced pressure. The residue was purified by column chromatography (SiO₂) on elution with dichloromethane (18 g, 90%).

MS: [M+NH₄⁺]=640

### Preparation of 5-[3-benzoic acid-4-(4-methyl phenyl sulfonamino)-benzyl]-2-(4-methyl phenyl sulfonamino)-benzoic acid methyl ester

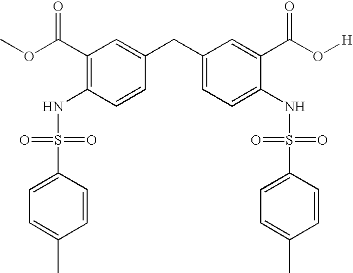

3,3′methylenebis[6-(4-methyl phenyl sulfonamino)-benzoic acid dimethyl ester] (1 g, 1.6 mmol) was dissolved in tetrahydrofurane (50 ml). Lithium hydroxide monohydrate (675 mg, 16 mmol) dissolved in water (50 ml) was added. The solution was stirred at room temperature for 16 hours. The solvents were evaporated under reduced pressure. The residue was extracted with dichloromethane/water, acidified with HCl until pH 7. The organic layer was dried over magnesium sulfate and evaporated under reduced pressure. The crude product was purified by column chromatography (SiO₂) on elution with dichloromethane:methanol 100:0 to 99:1 (70 mg, 7.2%).

MS: [M−H]⁻=607

### Preparation of 5-[3-Hydroxymethyl-4-(4-methyl phenyl sulfonamino)-benzyl]-2-(4-methyl phenyl sulfonamino)-benzoic acid methyl ester

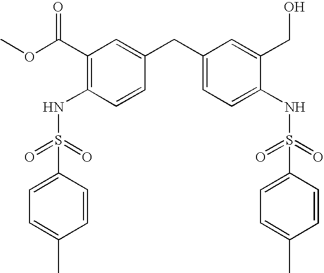

5-[3-benzoic acid-4-(4-methyl phenyl sulfonamino)-benzyl]-2-(4-methyl phenyl sulfonamino)-benzoic acid methyl ester (70 mg, 115 μmol) was dissolved in tetra-hydrofurane (10 ml, extra dry). The solution was flushed with nitrogen, sealed and cooled to 0° C. Borane-tetrahydrofurane complex (0.345 ml, 3.57 mmol) was added slowly. The solution was stirred at room temperature for 16 hours. The solution was slowly diluted with water and extracted with ethyl acetate. The organic layer was dried over magnesium sulfate and evaporated under reduced pressure. The crude product was used in the next step without further purification (30 mg, 44%).

MS: [M+NH₄⁺]=612

### Preparation of 5-[3-Hydroxymethyl-4-(4-methyl phenyl sulfonamino)-benzyl]-2-(4-methyl phenyl sulfonamino)-benzoic acid

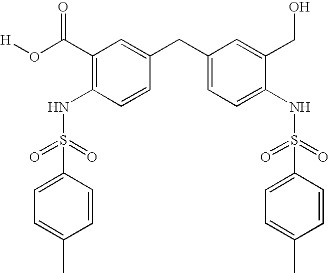

5-[3-Hydroxymethyl-4-(4-methyl phenyl sulfonamino)-benzyl]-2-(4-methyl phenyl sulfonamino)-benzoic acid methyl ester (30 mg, 50 μmol) was dissolved in tetrahydro-furane (5 ml). Lithium hydroxide monohydrate (22 mg, 504 μmol) dissolved in water (5 ml) was added. The solution was stirred at room temperature for 30 hours. The solvents were evaporated under reduced pressure. The residue was extracted with dichloromethane/water, acidified with HCl until pH 7. The organic layer was dried over magnesium sulfate and evaporated under reduced pressure. The crude product was purified by column chromatography (SiO₂) on elution with dichloromethane:methanol 100:0 to 95:5 (13 mg, 30.2%, purity (LC)=67.5%).

MS: [M−H]⁻=579

### Compound 16

### Preparation of 3,3′-methylenebis[6-(amino)-benzoic acid dibenzyl ester]

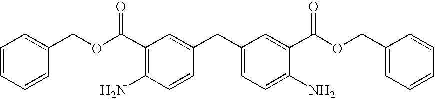

Benzyl anthranilate (5 g, 22 mmol), formaldehyde (0.820 ml, 11 mmol) and hydrogen chloride (4.6 ml, 25.5 mmol, 24% solution in 2-propanol) were dissolved in benzyl alcohol (20 ml). The solution was stirred at 65° C. for 1 hour. The solution was cooled down to room temperature, isopropyl ether was added and the mixture was stirred for 0.5 hours. The crystals were filtered off and dried in vacuo for 1 hour (5.350 g, 51.2%, purity (LC)=49.15%).

MS: [M+H]⁺=467

### Compound 17

### Preparation of 5-[3-Methyl-4-(4-methyl phenyl sulfonamino)-phenoxy]-2-(4-methyl phenyl sulfonamino)-benzoic acid

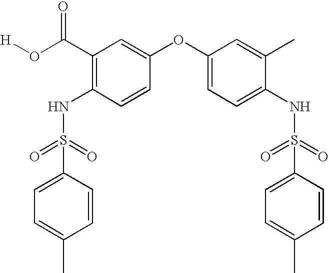

5-[3-Methyl-4-(4-methyl phenyl sulfonamino)-phenoxy]-2-(4-methyl phenyl sulfonamino)benzoic acid methyl ester (251 mg, 432 μmol) was dissolved in tetra-hydrofurane (50 ml).). A solution of lithium hydroxide monohydrate in water (50 ml, 0.4 M) was added to the tetrahydrofurane solution, resulting in an overall concentration of 0.2 M of lithium hydroxide. The solution was stirred at room temperature for 64 hours. The solvents were evaporated under reduced pressure. The residue was extracted with ethyl acetate/water, acidified with HCl until pH 7. The organic layer was dried over magnesium sulfate and evaporated under reduced pressure to obtain the desired product (232 mg, 92%, purity (LC)=97.6%).

MS: [M+H]⁺=566

MS: [M−H]⁻=564

### Compound 18

### Preparation of 5-[5-Fluoro-2-(4-methyl phenyl sulfonamino)-phenoxy]-2-(4-methyl phenyl sulfonamino)-benzoic acid

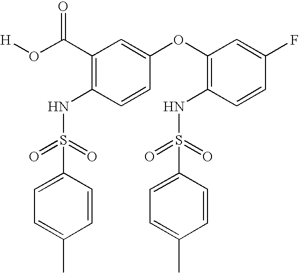

5-[5-Fluoro-2-(4-methyl phenyl sulfonamino)-phenoxy]-2-(4-methyl phenyl sulfonamino)benzoic acid methyl ester (27.19 mg, 47 μmol) was dissolved in tetra-hydrofurane (10 ml). A solution of lithium hydroxide monohydrate in water (10 ml, 0.4 M) was added to the tetrahydrofurane solution, resulting in an overall concentration of 0.2 M of lithium hydroxide. The solution was stirred at room temperature for 88 hours. The solvents were evaporated under reduced pressure. The residue was extracted with ethyl acetate/water, acidified with HCl until pH 7. The organic layer was dried over magnesium sulfate and evaporated under reduced pressure to obtain the desired product (26.3 mg, 95%, purity (LC)=95.8%).

MS: [M+H]⁺=570

MS: [M−H]⁻=568

### Compound 19

### Preparation of 5-Hydroxy-2-nitro-benzoic acid methyl ester

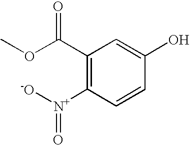

5-Hydroxy-2-nitro-benzoic acid (20 g, 109 mmol) was dissolved in methanol (700 ml) and thionylchloride (5.6 ml, 76 mmol) was slowly added. The solution was refluxed for 40 hours. The solvent was evaporated under reduced pressure, and the mixture was dissolved in ethyl acetate. The organic layer was washed with brine, dried over sodium sulfate and evaporated under reduced pressure. The crude product was used in the next step without further purification (21.5 g, 97.5%).

MS: [M−H]⁻=196

### Preparation of 2-Nitro-5-(4-nitro-phenoxy)-benzoic acid methyl ester

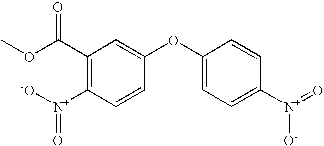

5-Hydroxy-2-nitro-benzoic acid methyl ester (10 g, 51 mmol), 1-Fluoro-4-nitrobenzene (7.2 g, 51 mmol), and potassium carbonate (8.4 g, 61 mmol) were dissolved in acetonitrile (700 ml). The solution was refluxed for 40 hours. The mixture was filtered and evaporated under reduced pressure. The residue was purified by column chromatography (SiO₂) on elution with dichloromethane:heptane (50:50) (3.7 g, 23%).

MS: [M+H]⁺=319

### Preparation of 2-Amino-5-(4-amino-phenoxy)-benzoic acid methyl ester

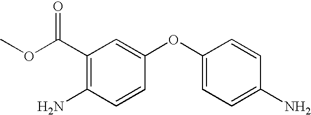

2-Nitro-5-(4-nitro-phenoxy)-benzoic acid methyl ester (3.70 g, 12 mmol) was dissolved in methanol (200 ml). 10% Palladium on carbon (300 mg, 282 μmol) was added and the dispersion was hydrogenated for 18 hours. The catalyst was filtered off and the solvent was evaporated under reduced pressure. The crude product was used in the next step without further purification (2.93 g, 92%).

MS: [M+H]⁺=259

### Preparation of 2-(4-methyl phenyl sulfonamino)-5-[4-(4-methyl phenyl sulfonamino)-phenoxy]-benzoic acid methyl ester

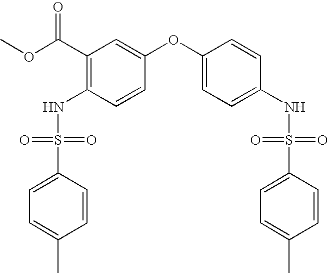

2-Amino-5-(4-amino-phenoxy)-benzoic acid methyl ester (200 mg, 774 μmol) and p-toluenesulfonyl chloride (303 mg, 1.587 mmol) were dissolved in pyridine (2 ml). The solution was stirred at room temperature for 40 hours. The solvent was evaporated under reduced pressure. The residue was dissolved in dichloromethane, washed with brine, dried over magnesium sulfate and evaporated under reduced pressure. The crude product was purified by column chromatography (SiO₂) on elution with dichloro-methane:methanol 99:1 (187.2 mg, 38.7%).

MS: [M+NH₄⁺]=584

### Preparation of 2-(4-methyl phenyl sulfonamino)-5-[4-(4-methyl phenyl sulfonamino)-phenoxy]-benzoic acid

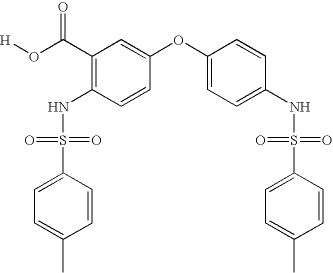

2-(4-methyl phenyl sulfonamino)-5-[4-(4-methyl phenyl sulfonamino)-phenoxy]-benzoic acid methyl ester (103 mg, 182 μmol) was dissolved in tetrahydrofurane (50 ml). A solution of lithium hydroxide monohydrate in water (50 ml, 0.4 M) was added to the tetrahydrofurane solution, resulting in an overall concentration of 0.2 M of lithium hydroxide. The solution was stirred at room temperature for 16 hours. The solvents were evaporated under reduced pressure. The residue was extracted with ethyl acetate/water, acidified with HCl until pH 7. The organic layer was dried over magnesium sulfate and evaporated under reduced pressure to obtain the desired product (70 mg, 63%, purity (LC)=90.3%).

MS: [M−2]=550

### Compound 20

### Preparation of 5-Chloro-2-nitro-benzoic acid methyl ester

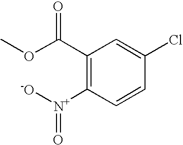

5-Chloro-2-nitro-benzoic acid (50 g, 247.5 mmol) was dissolved in methanol (700 ml), and thionylchloride was added slowly (12.65 ml, 173.3 mmol). The solution was refluxed for 40 hours. The solvent was evaporated under reduced pressure. The residue was dissolved in dichloromethane, washed with a sodium bicarbonate-solution, dried over sodium sulfate and evaporated under reduced pressure. The crude product was used in the next step without further purification (42.94 g, 80.46%).

MS: [M+H]⁺=216

### Preparation of 2-Nitro-5-(quinolin-6-yloxy)-benzoic acid methyl ester

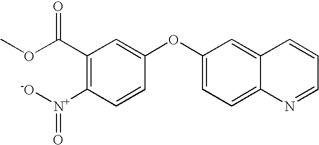

5-Chloro-2-nitro-benzoic acid methyl ester (2593.7 mg, 12.031 mmol), 6-hydroxy-quinoline (1782 mg, 12.031 mmol) and sodium carbonate were dissolved in DMF (100 ml). The solution was heated at 120° C. for 16 hours. The solution was filtered and evaporated under reduced pressure. The residue was purified by column chromatography (SiO₂) on elution with dichloromethane:methanol 100:0 to 99:1 (2467.7 mg, 57%).

MS: [M+30]⁺=324

### Preparation of 2-Amino-5-(1,2,3,4-tetrahydro-quinolin-6-yloxy)-benzoic acid methyl ester

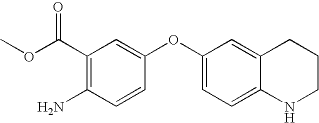

2-Nitro-5-(quinolin-6-yloxy)-benzoic acid methyl ester (2.4677 g, 7.6 mmol) was dissolved in methanol (50 ml). 10% Palladium on carbon (300 mg, 282 μmol) and 6N HCl in isopropanol (1.5 ml, 37 mmol) were added and the dispersion was hydrogenated for 18 hours. The catalyst was filtered off and the solvent was evaporated under reduced pressure. The crude product was used in the next step without further purification (2.477 g, 99%).

MS: [M+H]⁺=299

### Compound 21

### Preparation of 5-Chloro-2-nitro-benzoic acid methyl ester

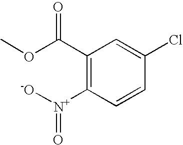

5-Chloro-2-nitro-benzoic acid (50 g, 247.5 mmol) was dissolved in methanol (700 ml), and thionylchloride was added slowly (12.65 ml, 173.3 mmol). The solution was refluxed for 40 hours. The solvent was evaporated under reduced pressure. The residue was dissolved in dichloromethane, washed with a sodium bicarbonate-solution, dried over sodium sulfate and evaporated under reduced pressure. The crude product was used in the next step without further purification (42.94 g, 80.46%).

MS: [M+H]⁺=216

### Preparation of 2-Nitro-5-(quinolin-6-yloxy)-benzoic acid methyl ester

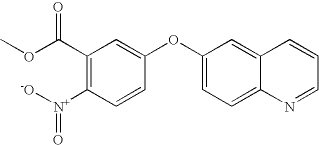

5-Chloro-2-nitro-benzoic acid methyl ester (2593.7 mg, 12.031 mmol), 6-hydroxy-quinoline (1782 mg, 12.031 mmol) and sodium carbonate were dissolved in DMF (100 ml). The solution was heated at 120° C. for 16 hours. The solution was filtered and evaporated under reduced pressure. The residue was purified by column chromatography (SiO₂) on elution with dichloromethane: methanol 100:0 to 99:1 (2467.7 mg, 57%, purity (LC)=82.34%).

MS: [M+30]+=324

### Compound 22

### Preparation of 3,3′-methylenebis[6-(amino)-benzoic acid dibenzyl ester]

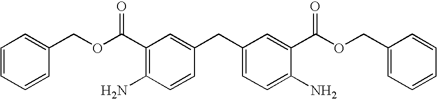

Benzyl anthranilate (5 g, 22 mmol), formaldehyde (0.820 ml, 11 mmol) and hydrogen chloride (4.6 ml, 27.5 mmol, 24% solution in 2-propanol) were dissolved in benzyl alcohol (20 ml). The solution was stirred at 65° C. for 1 hour. The solution was cooled down to room temperature, isopropyl ether was added and the mixture was stirred for 0.5 hours. The crystals were filtered off and dried in vacuo for 1 hour (5.350 g, 51.2%, purity (LC)=49.15%).

MS: [M+H]⁺=467

### Preparation of 3,3′-methylenebis[6-(4-methyl phenyl sulfonamino)-benzoic acid dibenzyl ester]

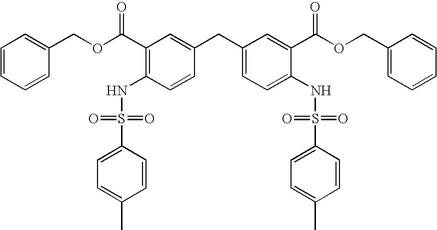

3,3′-methylenebis[6-(amino)-benzoic acid dibenzyl ester] (5.35 g, 10 mmol) and p-toluenesulfonyl chloride were dissolved in pyridine (20 ml). The solution was stirred at room temperature for 16 hours. The solvent was evaporated under reduced pressure. The residue was dissolved in dichloromethane and washed with diluted HCl. The organic layer was dried with magnesium sulfate and evaporated under reduced pressure. The crude product was purified by column chromatography (SiO₂) on elution with dichloromethane:heptane 7:3 (3.94 g, 46%, purity (LC)=90.44%).

MS: [M+NH₄⁺]=792

### Compound 23

### Preparation of 5-Hydroxy-2-nitro-benzoic acid methyl ester

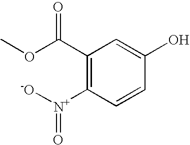

5-Hydroxy-2-nitro-benzoic acid (20 g, 109 mmol) was dissolved in methanol (700 ml) and thionylchloride (5.6 ml, 76 mmol) was slowly added. The solution was refluxed for 40 hours. The solvent was evaporated under reduced pressure, and the mixture was dissolved in ethyl acetate. The organic layer was washed with brine, dried over sodium sulfate and evaporated under reduced pressure. The crude product was used in the next step without further purification (21.5 g, 97.5%).

MS: [M−H]−=196

### Preparation of 2-Nitro-5-(4-nitro-phenoxy)-benzoic acid methyl ester

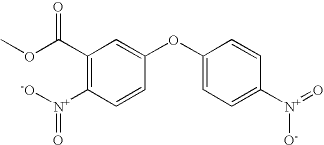

5-Hydroxy-2-nitro-benzoic acid methyl ester (10 g, 51 mmol), 1-Fluoro-4-nitrobenzene (7.2 g, 51 mmol), and potassium carbonate (8.4 g, 61 mmol) were dissolved in acetonitrile (700 ml). The solution was refluxed for 40 hours. The mixture was filtered and evaporated under reduced pressure. The residue was purified by column chromatography (SiO₂) on elution with dichloromethane:heptane (50:50) (3.7g, 23%).

MS: [M+H]⁺=319

### Preparation of 2-Amino-5-(4-amino-phenoxy)-benzoic acid methyl ester

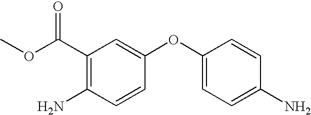

2-Nitro-5-(4-nitro-phenoxy)-benzoic acid methyl ester (3.70 g, 12 mmol) was dissolved in methanol (200 ml). 10% Palladium on carbon (300 mg, 282 μmol) was added and the dispersion was hydrogenated for 18 hours. The catalyst was filtered off and the solvent was evaporated under reduced pressure. The crude product was used in the next step without further purification (2.93 g, 92%).

MS: [M+H]⁺=259

### Preparation of 2-(4-methyl phenyl sulfonamino)-5-[4-(4-methyl phenyl sulfonamino)-phenoxy]-benzoic acid methyl ester

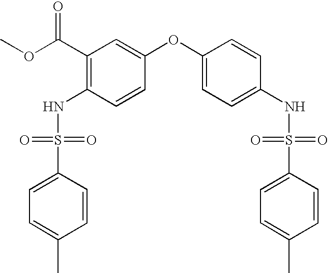

2-Amino-5-(4-amino-phenoxy)-benzoic acid methyl ester (200 mg, 774 μmol) and p-toluenesulfonyl chloride (303 mg, 1.587 mmol) were dissolved in pyridine (2 ml). The solution was stirred at room temperature for 40 hours. The solvent was evaporated under reduced pressure. The residue was dissolved in dichloromethane, washed with brine, dried over magnesium sulfate and evaporated under reduced pressure. The crude product was purified by column chromatography (SiO₂) on elution with dichloro-methane:methanol 99:1 (187.2 mg, 38.7%, purity (LC)=90.82%).

MS: M+H₂O]=584

### Compound 24

### Preparation of Thiophen-2-ylmethyl-thiourea

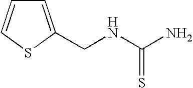

2-Isothiocyanatomethyl-thiophene (8 ml) was added drop wise to a mixture of 28% ammonium hydroxide in water (24 ml, 166 mmol) and ethanol (8 ml) at 0° C. The solution was stirred at room temperature for 16 hours. The solvent was evaporated under reduced pressure. The crude product was used in the next step without further purification (10 g, %).

### Preparation of (4-Phenyl-thiazol-2-yl)-thiophen-2-ylmethyl-amine

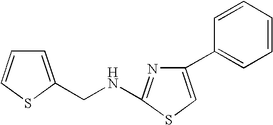

Thiophen-2-ylmethyl-thiourea (172 mg, 1 mmol) and 2-Bromo-1-phenyl-ethanone (199 mg, 1 mmol) were dissolved in ethanol (5 ml). The solution was heated at 50° C. for 16 hours. The solvent was evaporated under reduced pressure. The residue was dissolved in dichloromethane and washed with a solution of potassium carbonate in water. The organic layer was dried over magnesium sulfate and evaporated under reduced pressure. The residue was purified by column chromatography (SiO₂) to obtain the desired product (93 mg, %).

MS: [M]=272

### Compound 25

### Preparation of 3,3′-methylenebis[6-(amino)-benzoic acid]

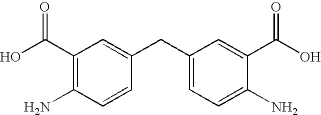

Anthranilic acid (10 g, 73.2 mmol) was dissolved in water (400 ml). Formaldehyde 37% solution in water (3 g, 36.8 mmol) and HCl 37% solution in water (7.2 g, 73.2 mmol) were added. The solution was stirred at 70° C. for 40 hours. The dispersion was filtered and the filtrate was evaporated under reduced pressure. The crude product was used in the next step without further purification (7.8 g, 58%).

MS: [M−2H]⁻=284

### Preparation of 2-(4-methyl phenyl sulfonamino)-5-[4-(4-methyl phenyl sulfonamino)-benzyl]-benzoic acid

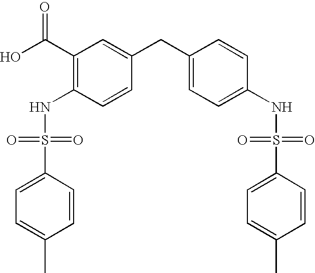

3,3′-methylenebis[6-(amino)-benzoic acid] (1 g, 3.5 mmol) was dissolved in tetra-hydrofurane (50 ml). Sodium bicarbonate (1.8 g, 21.8 mmol) dissolved in water (25 ml) and p-toluenesulfonyl chloride (1.5 g, 8.1 mmol) were added. The solution was stirred at room temperature for 30 minutes. The solvent was evaporated under reduced pressure. The residue was dissolved in water and acidified with HCl 37% solution in water. The precipitate was filtered off and dried in vacuo. The product was purified by column chromatography (SiO₂) on elution with dichloromethane:methanol 96:4 (0.529 g, 25.77%, purity (LC)=96.14%).

MS: [M−H]⁻=593

### Compound 26

### Preparation of 3,3′-methylenebis[6-(amino)-benzoic acid dimethyl ester]

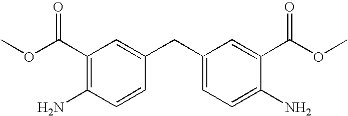

Methyl anthranilate (15 g, 99 mmol), formaldehyde (4.5 ml, 50 mmol) and HCl 37% solution in water (8.6 ml, 103 mmol) were dissolved in methanol (250 ml). The solution was refluxed for 16 hours. The solution was cooled down to room temperature and isopropyl ether was added. The precipitate was filtered off and dried in vacuo (12 g, 77%).

MS: [M+H]⁺=315

### Preparation of 3,3′-methylenebis[6-(toluene-4-sulfonamino)-benzoic acid dimethyl ester]

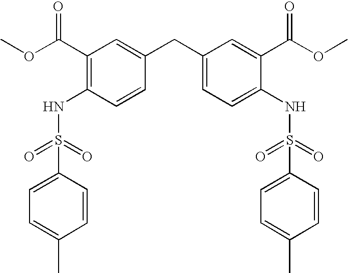

3,3′-methylenebis[6-(amino)-benzoic acid dimethyl ester] (2 g, 6 4 mmol) and p-toluenesulfonyl chloride (3.7 g, 6.4 mmol) were dissolved in pyridine (50 ml). The solution was stirred for 16 hours. The solvent was evaporated under reduced pressure. The residue was dissolved in dichloromethane, washed with diluted HCl and dried over magnesium sulfate. The solvent was evaporated under reduced pressure. The crude product was purified by column chromatography (SiO₂) on elution with dichloro-methane (2.4 g, 58.74%, purity (LC)=97.36%).

MS: [M+NH₄⁺]=640

### Compound 27

### Preparation of 3,3′-methylenebis[6-(amino)-benzoic acid dimethyl ester]

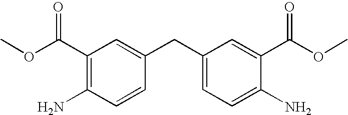

Methyl anthranilate (15 g, 99 mmol), formaldehyde (4.5 ml, 50 mmol) and HCl 37% solution in water (8.6 ml, 103 mmol) were dissolved in methanol (250 ml). The solution was refluxed for 16 hours. The solution was cooled down to room temperature and isopropyl ether was added. The precipitate was filtered off and dried in vacuo (12 g, 77%).

MS: [M+H]+=315

### Preparation of 3,3′-methylenebis-[6-(toluene-4-sulfonamino)-benzoic acid dimethyl ester]

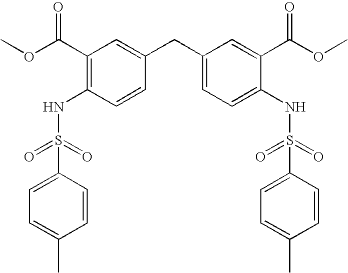

3,3′-methylenebis[6-(amino)-benzoic acid dimethyl ester] (2 g, 6.4 mmol) and p-toluenesulfonyl chloride (3.7 g, 6.4 mmol) were dissolved in pyridine (50 ml). The solution was stirred for 16 hours. The solvent was evaporated under reduced pressure. The residue was dissolved in dichloromethane, washed with diluted HCl and dried over magnesium sulfate. The solvent was evaporated under reduced pressure. The crude product was purified by column chromatography (SiO₂) on elution with dichloro-methane (2.4 g, 58.74%).

MS: [M+NH₄⁺]=640

### Preparation of 5-[3-Carboxy-4-(4-methyl phenyl sulfonamino)-benzyl]-2-(4-methyl phenyl sulfonamino)-benzoic acid methyl ester

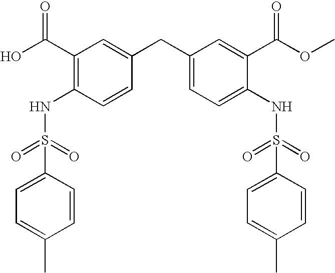

3,3′-methylenebis-[6-(toluene-4-sulfonamino)-benzoic acid dimethyl ester] (593 mg, 0.95 mmol) and lithium hydroxide monohydrate (20 mg, 0.48 mmol) were dissolved in a tetrahydrofurane:water 50:50 mixture (50 ml). The solution was stirred at room temperature for 5.5 hours. The solution was acidified with HCl 37% in water to pH 3. The solvent was evaporated partly. The product was extracted with dichloromethane, dried over sodium sulfate and the solvent was evaporated under reduced pressure. The crude product was purified by column chromatography (SiO₂) to obtain the pure compound (35 mg, 6%, purity (LC)=99.59%).

MS: [M−H]⁻=607

MS: [M+H]⁺=609

### Compound 28

### Preparation of 3,3′-methylenebis[6-(amino)-benzoic acid dimethyl ester]

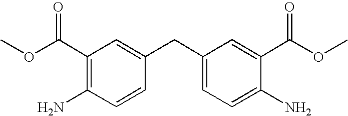

Methyl anthranilate (15 g, 99 mmol), formaldehyde (4.5 ml, 50 mmol) and HCl 37% solution in water (8.6 ml, 103 mmol) were dissolved in methanol (250 ml). The solution was refluxed for 16 hours. The solution was cooled down to room temperature and isopropyl ether was added. The precipitate was filtered off and dried in vacuo (12 g, 77%).

MS: [M+H]⁺=315

### Preparation of 3,3′-methylenebis[6-(4-methyl phenyl sulfonamino)-benzoic acid dimethyl ester]

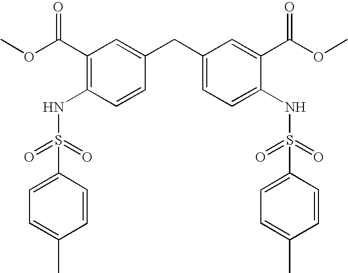

3,3′-methylenebis[6-(amino)-benzoic acid dimethyl ester] (2 g, 6.4 mmol) and p-toluenesulfonyl chloride (3.7 g, 19.2 mmol) were dissolved in pyridine (50 ml). The solution was stirred for 16 hours. The solvent was evaporated under reduced pressure. The residue was dissolved in dichloromethane, washed with diluted HCl and dried over magnesium sulfate. The solvent was evaporated under reduced pressure. The crude product was purified by column chromatography (SiO₂) on elution with dichloro-methane (2.4 g, 58.74%).

MS: [M+NH₄⁺]=640

### Preparation of N,N′-[methylenebis[2-(hydroxydiphenylmethyl)-4,1-phenylene]]bis[4-methyl-(benzenesulfonamide)]

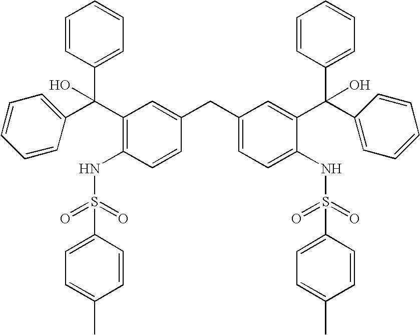

3,3′-methylenebis[6-(4-methyl phenyl sulfonamino)-benzoic acid dimethyl ester] (346 mg, 0.556 mmol) and phenylmagnesium chloride (760 mg, 5.56 mmol) were dissolved in tetrahydrofurane (100 ml). The solution was stirred at room temperature for 3 hours. The solution was diluted with water and acidified with HCl 37% in water. The organic layer was evaporated under reduced pressure. The crude product was purified by column chromatography (SiO₂) on elution with dichloromethane:methanol 100:0 to 98:2 (405 mg, 83.7%, purity (LC)=91.44%).

MS: [M+17]=888

### Compound 29

### Preparation of 5-Mercapto-2-nitro-benzoic acid

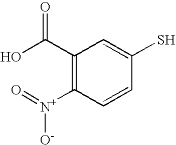

5,5′-Dithiobis(2-nitrobenzoic acid) (4.025 g, 10.155 mmol) was dissolved in ethanol (300 ml). The solution was heated to 70° C. and sodium borohydride (1.537 g, 40.62 mmol) was added slowly. The solution was refluxed for 1 hour. The mixture was diluted with water (250 ml), acidified with HCl 37% in water and extracted with dichloromethane. The organic layer was dried over magnesium sulfate and evaporated under reduced pressure. The crude product was used in the next step without further purification (4 g, 99%).

MS: [M−H]⁻=198

### Preparation of 5-Mercapto-2-nitro-benzoic acid methyl ester

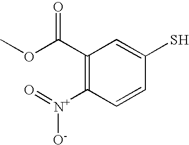

5-Mercapto-2-nitro-benzoic acid (4.045 g, 20.3 mmol) and sulfuric acid (1.992 g, 20.3 mmol) were dissolved in methanol (300 ml). The solution was refluxed for 16 hours. The solvent was evaporated under reduced pressure. The product was purified over a silica filter (720 mg, 16.6%).

### Preparation of 2-Nitro-5-(4-nitro-phenylsulfanyl)-benzoic acid methyl ester

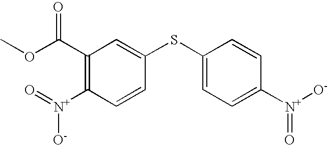

5-Mercapto-2-nitro-benzoic acid methyl ester (720 mg, 3.38 mmol), 1-fluoro-4-nitro-benzene (476 mg, 3.38 mmol) and potassium carbonate (583 mg, 4.225 mmol) were dissolved in acetonitrile (150 ml). The solution was refluxed for 16 hours. The mixture was filtered and the solvent was evaporated under reduced pressure (720 mg, 63.8%).

### Preparation of 2-Amino-5-(4-amino-phenylsulfanyl)-benzoic acid methyl ester

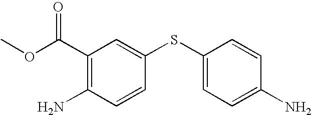

2-Nitro-5-(4-nitro-phenylsulfanyl)-benzoic acid methyl ester (720 mg, 2.154 mmol) was dissolved in methanol (75 ml) and tetrahydrofurane (75 ml). Palladium on carbon 10% (150 mg, 14 mmol) was added and the dispersion was hydrogenated for 1 hour. The catalyst was filtered off and the solvent was evaporated under reduced pressure. The crude product was purified by column chromatography (SiO₂) on elution with dichloromethane:methanol 100:0 to 99.5:0.5 (160 mg, 27%).

MS: [M+H]⁺=275

### Preparation of 2-(4-methyl phenyl sulfonamino)-5-[4-(4-methyl phenyl sulfonamino)-phenylsulfanyl]-benzoic acid methyl ester

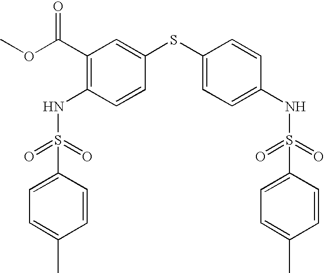

2-Amino-5-(4-amino-phenylsulfanyl)-benzoic acid methyl ester (160 mg, 583 μmol) and p-toluenesulfonyl chloride (334 mg, 1.75 mmol) were dissolved in pyridine (20 ml). The solution was stirred at room temperature for 20 hours. The solvent was evaporated under reduced pressure. The crude product was extracted with dichloro-methane, washed with diluted HCl and dried over sodium sulfate. The crude product was purified by column chromatography (SiO₂) on elution with dichloromethane:methanol 100:0 to 99.5:0.5 (20 mg, 5.9%).

MS: [M+17]=599

### Preparation of 2-(4-methyl phenyl sulfonamino)-5-[4-(4-methyl phenyl sulfonamino)-phenylsulfanyl]-benzoic acid

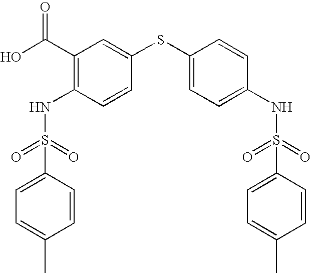

2-(4-methyl phenyl sulfonamino)-5-[4-(4-methyl phenyl sulfonamino)-phenylsulfanyl]-benzoic acid methyl ester (20 mg, 34 μmol) and sodium hydroxide (1.4 mg, 34 μmol) were dissolved in tetrahydrofurane (10 ml) and water (10 ml). The solution was stirred at room temperature for 16 hours. The solvent was evaporated under reduced pressure. The crude product was dissolved in dichloromethane, acidified with HCl and dried over magnesium sulfate. The solvent was evaporated under reduced pressure to obtain the desired product (10 mg, 32%, purity (LC)=62.93%).

MS: [M−2H]⁻=566

### Compound 30

### Preparation of 3,3′-methylenebis[6-(amino)-benzoic acid]

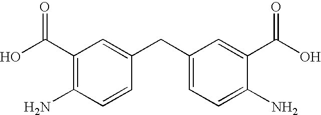

Anthranilic acid (10 g, 73.2 mmol) was dissolved in water (400 ml). Formaldehyde 37% solution in water (3 g, 36.8 mmol) and HCl 37% solution in water (7.2 g, 73.2 mmol) were added. The solution was stirred at 70° C. for 40 hours. The dispersion was filtered and the solvent was evaporated under reduced pressure. The crude product was used in the next step without further purification (7.8 g, 58%).

MS: [M−2H]⁻=284

### Preparation of 2-[3-(4-Methanesulfonyl-phenyl)-acryloylamino]-5-{4-[3-(4-sulfonyl-phenyl)-acryloylamino]-benzyl}-benzoic acid

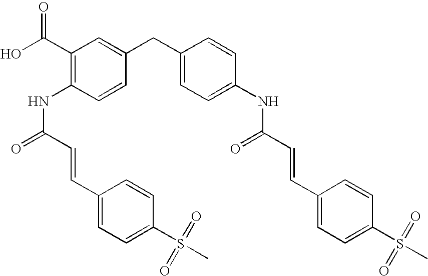

3,3′-methylenebis[6-(amino)-benzoic acid] (97.3 mg, 0.34 mmol), 3-(4-methane-sulfonylphenyl)-acryloyl chloride (183 mg, 2.2 mmol) and sodium bicarbonate (192.9 mg, 0.79 mmol) were dissolved in tetrahydrofurane (6 ml). The solution was stirred at room temperature for 2 hours. The solvent was evaporated under reduced pressure. The mixture was acidified with HCl, precipitation was filtered off and dried in vacuo (7.9 mg, 3.2%, purity (LC)=87.83%).

MS: [M+H]⁺=659

### Compound 31

### Preparation of 3-hydroxy-5-nitrobenzoic acid methyl ester

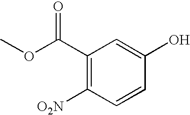

At RT, SOCL2 (13.0 g, 0.109 mol) was added gradually to a solution of commercially available 3-hydroxy-5-nitrobenzoic acid (20.0 g, 0.109 mol) in MeOH (500 mL). The reaction was warmed at reflux for 72 h. The reaction was cooled to RT and evaporated to dryness in vacuo. The residue was dissolved in EtOAc (200 mL) and washed with aq. saturated NaHCO3 (200 mL). The aqueous-layer was extracted with EtOAc (2×200 mL). The combined EtOAc-extracts were washed with brine (1×200 mL). The organic layer was dried (Na₂SO₄), filtered, and evaporated to dryness to obtain 3-hydroxy-5-nitrobenzoic acid methyl ester (16.9 g, 79%, purity (LC)>95%).

MS: [M−H]⁻=196.

### Preparation of 5-amino-3-hydroxybenzoic acid methyl ester

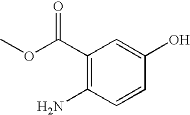

At RT, 10% Pd—C (45 mg) was added to a solution of 3-hydroxy-5-nitrobenzoic acid methyl ester (5.61 g, 28.5 mmol) in methanol (60 mL). The reaction was placed under 1 atmosphere of hydrogen and was stirred at RT for 3 hours. An additional amount of 10% Pd—C (140 mg) was added and the reaction was continued for 19 hours. The reaction was filtered over Kiezelguhr and the filtrate was evaporated to dryness in vacuo to afford 5-amino-3-hydroxybenzoic acid methyl ester as a yellow solid (3.89 g, 82%, purity (LC)=90%).

MS: [M+H]⁻⁺=168

### Preparation of methyl 5-hydroxy-2-[(4-methylphenyl)sulfonyl]aminobenzoate

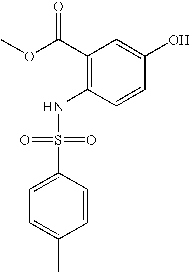

At RT, tosyl chloride (4.63 g, 24.3 mmol) was added to a suspension of 5-amino-3-hydroxybenzoic acid methyl ester (3.89 g, 23.3 mmol) and pyridine (3.70 g, 46.8 mmol) in DCM/MeOH (50 mL). The reaction was stirred at RT for 1 hour. The reaction mixture was evaporated to dryness in vacuo. The residue was dissolved in DCM (100 mL) and washed with aq. 0.5 N KHSO₄ (2×50 mL). The aqueous-layers were extracted once more with DCM (100 mL). The combined DCM-extracts were dried (Na₂SO₄), filtered, and evaporated to dryness in vacuo. The crude product was recrystallised from MeOH to obtain methyl 5-hydroxy-2-[(4-methylphenyl)sulfonyl]-aminobenzoate as a yellow solid (4.41 g, 59%, purity (LC)>95%).

MS: [M+H]⁻⁺=320

### Preparation of 5-(3-methyl-4-nitro-phenoxy)-2-(toluene-4-sulfonylamino)-benzoic acid methyl ester

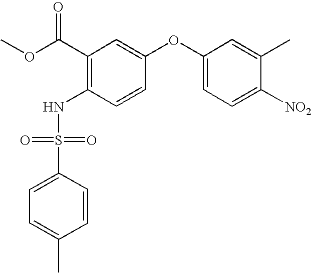

A suspension of methyl 5-hydroxy-2-[(4-methylphenyl)sulfonyl]aminobenzoate (600 mg, 1.87 mmol), commercially available 5-fluoro-2-nitrotoluene (290 mg, 1.87 mmol) and potassium carbonate (645 mg, 4.67 mmol) in dimethylsulfoxide (25 mL) was stirred at 80° C. After 4 hours, the reaction was diluted with water (250 mL) and extracted with ethyl acetate (3×100 mL). The combined extracts were washed with aqueous saturated ammonium chloride (2×100 mL) and brine (2×100 mL), dried over anhydrous sodium sulfate and evaporated in vacuo. The oily residue was stirred in ethanol (90 mL) for 0.5 hours and precipitation of a solid occurred. The suspension was stirred 0.5 hours at 0° C. and filtered. The brown solid was dried in vacuo at 40° C. for 16 hours to afford the desired product (666 mg, 78%, purity (LC) 94%).

MS: [M+H]⁺=457

### Preparation of 5-(4-amino-3-methyl-phenoxy)-2-(toluene-4-sulfonylamino)-benzoic acid methyl ester

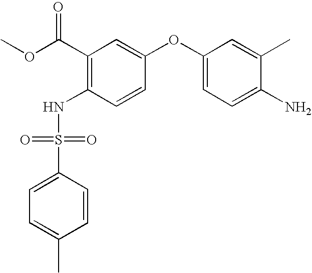

A suspension of 5-(3-Methyl-4-nitro-phenoxy)-2-(toluene-4-sulfonylamino)benzoic acid methyl ester (666 mg, 1.46 mmol) and 10% palladium on activated carbon (77 mg, 0.07 mmol) in tetrahydrofuran (5 mL) and methanol (5 mL) was stirred at room temperature under 1 atmosphere of hydrogen. After 4 hours the reaction was filtered over Kieselguhr and evaporated under reduced pressure to afford the desired product (600 mg, 96%, purity n.d.)

### Preparation of 5-[3-methyl-4-(toluene-4-sulfonylamino)-phenoxy]-2-(toluene-4-sulfonylamino)-benzoic acid methyl ester

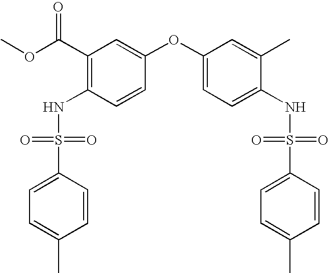

A solution of 5-(4-Amino-3-methyl-phenoxy)-2-(toluene-4-sulfonylamino)-benzoic acid methyl ester (600 mg, 1.41 mmol), p-toluenesulfonylchloride (300 mg, 1.55 mmol) and pyridine (220 mg, 2.81 mmol) in dichloromethane (10 mL) was stirred for 16 hours at room temperature. The reaction was diluted with dichloromethane (50 mL) and washed with aqueous 0.5N potassium hydrogen sulfate (30 mL), saturated sodium hydrogen carbonate (30 mL) and dried over anhydrous sodium sulfate. After concentration under reduced pressure the residue was triturated in boiling DIPE containing 5% methanol. After cooling to 0° C., the crystals were filtered and dried in vacuo for 16 hours (677 mg, 83%, purity (LC)>95%).

MS: [M−H]⁻=579

NMR: ¹H (CDCl₃): δ 10.31 (s, 1H), 7.71 (d, J=8.32 Hz, 2H), 7.68 (d, J=9.08 Hz, 1H), 7.59 (d, J=8.08 Hz, 2H), 7.49 (d, J=2.00 Hz, 1H), 7.24 (dd, J=3.28 Hz and J=8.08 Hz, 4H), 7.17-7.15 (m, 1H), 7.10 (dd, J=2.76 Hz and J=9.08 Hz, 1H), 6.68-6.65 (m, 2H), 6.15 (s, 1H), 3.83 (s, 3H), 2.41 (s, 3H), 2.38 (s, 3H)

### Preparation of 5-[3-methyl-4-(toluene-4-sulfonylamino)-phenoxy]-2-(toluene-4-sulfonylamino)-benzoic acid

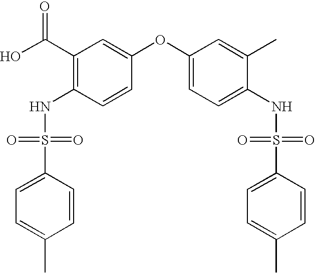

A mixture of 5-[3-Methyl-4-(toluene-4-sulfonylamino)-phenoxy]-2-(toluene-4-sulfonylamino)-benzoic acid methyl ester (300 mg, 0.517 mmol), aq. 1N LiOH (4.4 mL, 4.28 mmol) and THF (4 mL) was stirred for 16 hours at room temperature. The reaction was acidified to pH=1 with aq. 2N HCl and the THF was evaporated under reduced pressure. The suspension was extracted with DCM (2×50 mL) and the extracts were dried over anhydrous sodium sulfate. Concentration of the organic solution afforded the desired compound(140 mg, 48%, purity (LC)>95%).

MS: [M−H]⁻=565

NMR: ¹H (DMSO-d6): δ 10.85 (bs, 1H), 9.47 (s, 1H), 7.66 (d, J=8.08 Hz, 2H), 7.51 (t, J=7.82 Hz, 3H) 7.37-7.31 (m, 6H), 7.25 (dd, J=3.00 Hz and 8.84 Hz, 1H), 6.92 (d, J=8.60 Hz, 1H), 6.77 (d, J=2.28, 1H), 6.71 (dd, J=2.52 Hz and 8.60 Hz, 1H), 2.36 (s, 3H), 2.34 (s, 3H), 1.89 (s, 3H)

### Compound 32

### Preparation of 5-[3-trifluoromethyl-4-(toluene-4-sulfonylamino)-phenoxy]-2-(toluene-4-sulfonylamino)-benzoic acid

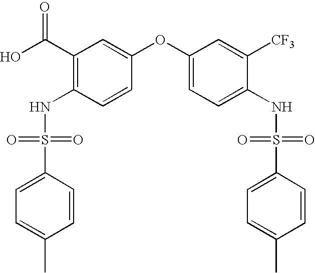

A solution of 5-[3-trifluoromethyl-4-(toluene-4-sulfonylamino)-phenoxy]-2-(toluene-4-sulfonylamino)-benzoic acid methyl ester (444 mg, 0.699 mmol) in aq. 0.5 M LiOH (5 mL) and THF (5 mL) was heated at 60° C. for 3 hours. The reaction mixture was cooled to RT and THF was removed by evaporation in vacuo. The residue was acidified by addition of aq. 1 N HCl (20 mL). The suspension was extracted with DCM (3×30 mL). The combined DCM-layers were dried (Na₂SO₄), filtered, and evaporated to dryness in vacuo. The crude product was purified by flash column chromatography (DCM/MeOH: 100/1 to 100/3, +1% of AcOH) and then by reversed-phase column chromatography (H₂O to H₂O/CH3CN: 1/1+1% Et₃N) to obtain the desired product as an aqueous solution. The aqueous solution was acidified to pH=1 by addition of aq. 1 M HCl and extracted with DCM. The combined DCM-extracts were dried (Na₂SO₄), filtered, and evaporated to dryness in vacuo to obtain the desired product as a white foam (196 mg, 45%, purity (LC)>95%).

MS: [M−H]⁻=619

NMR: ¹H (DMSO-d6): δ 10.98 (s, 1H), 9.85 (s, 1H), 7.69-7.62 (m, 4H), 7.55 (d, J=9.09 Hz, 1H), 7.46 (d, J=3.03 Hz, 1H), 7.42-7.32 (m, 5H), 7.23 (d, J=3.04 Hz, 1H), 7.13 (dd, J=2.78 Hz and J=8.59 Hz, 1H), 6.94 (d, J=8.84 Hz, 1H), 2.39 (s, 3H), 2.34 (s, 3H).

### Preparation of 5-[3-trifluoromethyl-4-(toluene-4-sulfonylamino)-phenoxy]-2-(toluene-4-sulfonylamino)-benzoic acid methyl ester

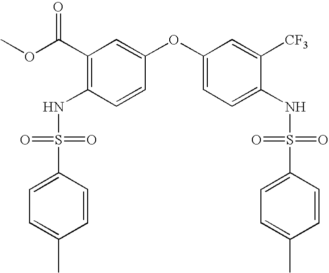

A solution of 5-(4-Amino-3-trifluoromethyl-phenoxy)-2-(toluene-4-sulfonylamino)-benzoic acid methyl ester (661 mg, 1.38 mmol), tosyl chloride (385 mg, 2.02 mmol), and pyridine (236 mg, 2.98 mmol) in DCM (7 mL) was stirred at reflux for 43 hours. The reaction mixture was diluted with DCM (50 mL) and washed with aq. 0.5 N KHSO₄ (100 mL) and aq. sat. NaHCO3 (100 mL). Aqueous-layers were extracted with DCM (1×30 mL). The combined DCM-layers were dried (Na₂SO₄), filtered, and evaporated to dryness in vacuo. The crude product was purified by flash column chromatography (EtOAc/heptane: 1/3) to obtain the desired product (purity (LC)=89%). The product was crystallized twice from EtOAc/heptane to afford 5-[3-trifluoro-methyl-4-(toluene-4-sulfonylamino)-phenoxy]-2-(toluene-4-sulfonylamino)-benzoic acid methyl ester as white crystals (474 mg, 56%, purity (LC)>95%).

MS: [M−H]⁻=633.

NMR: ¹H (CDCl3): δ 10.37 (s, 1H), 7.76-7.70 (m, 4H), 7.63 (d, J=8.36 Hz, 2H), 7.52 (d, J=3.04 Hz, 1H), 7.27-7.21 (m, 4H), 7.11 (dd, J=2.80 Hz and J=9.12 Hz, 1H), 7.04-6.99 (m, 2H), 3.84 (s, 3H), 2.40 (s, 3H), 2.38 (s, 3H).

### Preparation of 5-(4-amino-3-trifluoromethyl-phenoxy)-2-(toluene-4-sulfonylamino)-benzoic acid methyl ester

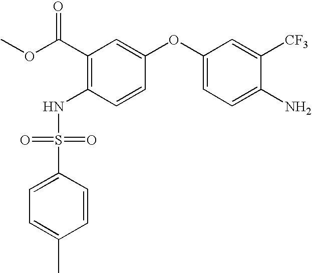

At RT, 10% Pd—C (37 mg) was added to a suspension of 5-(4-nitro-3-trifluoromethyl-phenoxy)-2-(toluene-4-sulfonylamino)-benzoic acid methyl ester (710 mg, 1.39 mmol) in THF/MeOH: 1/1 (10 mL). The reaction was placed under 1 atmosphere of hydrogen and stirred at RT for 4 hours. The reaction mixture was filtered over Kiezelguhr. The residue was rinsed with THF (30 mL) and MeOH (mL). The filtrate was evaporated to dryness to obtain 5-(4-amino-3-trifluoromethyl-phenoxy)-2-(toluene-4-sulfonylamino)-benzoic acid methyl ester_(661 mg, 99%, purity (LC)>95%).

MS: [M+H]⁺=481.

### Preparation of 5-(4-nitro-3-trifluoromethyl-phenoxy)-2-(toluene-4-sulfonylamino)-benzoic acid methyl ester

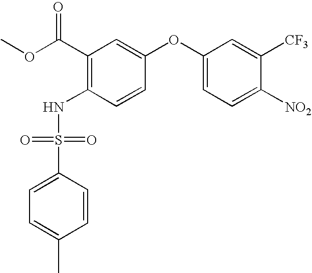

A mixture of methyl 5-hydroxy-2-[(4-methylphenyl)sulfonyl]aminobenzoate (499 mg, 1.55 mmol), commercially available 5-fluoro-2-nitrobenzotrifluoride (353 mg, 1.69 mmol), and potassium carbonate (428 mg, 3.10 mmol) in DMF (7 mL) was stirred at 80° C. for 2 hours. The reaction mixture was cooled to RT, diluted with EtOAc (50 mL) and washed with brine (3×50 mL). The aqueous-layers were extracted with EtOAc (1×50 mL). The EtOAc-extracts were dried (Na₂SO₄), filtered, and evaporated to dryness in vacuo. The crude product was triturated with EtOH (5 mL) to obtain the desired product (710 mg, 90%, purity (LC)>95%).

MS: [M−H]⁻=509.

### Compound 33

### Preparation of 5-[3-Fluoro-4-(toluene-4-sulfonylamino)-phenoxy]-2-(toluene-4-sulfonylamino)-benzoic acid

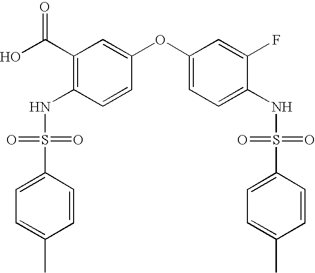

A solution of 5-[3-Fluoro-4-(toluene-4-sulfonylamino)-phenoxy]-2-(toluene-4-sulfonylamino)-benzoic acid methyl ester (364 mg, 0.622 mmol) in aq. 0.5 M LiOH (6 mL) and THF (6 mL) was stirred at 60° C. for 3 hours. The reaction was cooled to RT and evaporated in vacuo to remove THF. The residue was acidified with aq. 2 M HCl (6 mL). The aqueous suspension was extracted with DCM (2×20 mL). The combined DCM-extracts were dried (Na₂SO₄), filtered, and evaporated to dryness to obtain a white solid in a yield of 370 mg. The crude product was triturated with heptane to give the desired product as a white solid (319 mg, 90%, purity (LC)>95%).

MS: [M−H]⁻=569.

NMR: ¹H (DMSO-d6): δ 11.05 (bs, 1H), 9.98 (s, 1H), 7.67 (d, J=8.32 Hz, 2H), 7.57 (d, J=8.36 Hz, 2H), 7.52 (d, J=8.84 Hz, 1H), 7.39-7.33 (m, 5H), 7.29 (dd, J=2.76 Hz and J=8.84 Hz, 1H), 7.17 (t, J=8.80 Hz, 1H), 6.86 (dd, J=2.52 Hz and 11.1 Hz, 1H), 6.72 (dd, J=2.04 Hz and J=8.84 Hz, 1H), 2.36 (s, 3H), 2.34 (s, 3H).

### Preparation of 5-[5-fluoro-2-(toluene-4-sulfonylamino)-phenoxy]-2-(toluene-4-sulfonylamino)-benzoic acid:

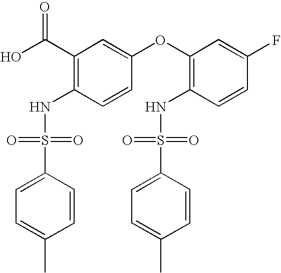

A solution of 5-[5-fluoro-2-(toluene-4-sulfonylamino)-phenoxy]-2-(toluene-4-sulfonyl-amino)-benzoic acid methyl ester (540 mg, 0.924 mmol) in aq. 0.5 M LiOH (10 mL) and THF (10 mL) was stirred at 60° C. for 3 hours. The reaction was cooled to RT and evaporated in vacuo to remove THF. The residue was acidified with aq. 2 M HCl (10 mL). The aqueous suspension was extracted with DCM (2×20 mL). The combined DCM-extracts were dried (Na₂SO₄), filtered, and evaporated to dryness to obtain the desired product as a white foam (514 mg, 97%, purity (LC)>95%).

MS: [M−H]⁻=569.

NMR: ¹H (DMSO-d6): δ 9.84 (s, 1H), 7.69 (d, J=8.32 Hz, 2H), 7.49-7.44 (m, 3H), 7.41-7.35 (m, 3H), 7.13 (d, J=8.08 Hz, 2H), 6.99-6.91 (m, 3H), 6.54 (dd, J=2.76 Hz, 1H), 2.35 (s, 3H), 2.23 (s, 3H).

### Preparation of 5-[3-Fluoro-4-(toluene-4-sulfonylamino)-phenoxy]-2-(toluene-4-sulfonylamino)-benzoic acid methyl ester

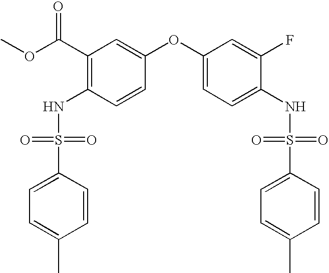

A mixture of 5-(4-Amino-3-fluoro-phenoxy)-2-(toluene-4-sulfonylamino)-benzoic acid methyl ester and 5-(2-Amino-5-fluoro-phenoxy)-2-(toluene-4-sulfonylamino)-benzoic acid methyl ester (1.55 g, 3.60 mmol), tosyl chloride (854 mg, 4.80 mmol), and pyridine (570 mg, 7.21 mmol) in DCM (10 mL) was stirred at reflux for 20 hours. The reaction was diluted with CH₂Cl₂ (30 mL) and washed with aq. 0.5 M KHSO₄ (30 mL) and aq. sat. NaHCO₃ (50 mL). The aqueous-layer were extracted once more with CH₂CL₂ (30 mL). The combined CH₂CL₂-layers were dried (Na₂SO₄), filtered, and evaporated to dryness in vacuo. The crude product was purified by flash column chromatography (EtOAc/heptane: 1/4 to 1/3) to obtain 5-[3-fluoro-4-(toluene-4-sulfonylamino)-phenoxy]-2-(toluene-4-sulfonylamino)-benzoic acid methyl ester (602 mg, 29%) and 5-[5-fluoro-2-(toluene-4-sulfonylamino)-phenoxy]-2-(toluene-4-sulfonylamino)-benzoic acid methyl ester (1.01 g, 48%). 5-[3-fluoro-4-(toluene-4-sulfonylamino)-phenoxy]-2-(toluene-4-sulfonylamino)-benzoic acid methyl ester was recrystallized from EtOAc/heptane to afford the desired product as a small white crystals (486 mg, 23%, purity (LC)>95%).

5-[3-fluoro-4-(toluene-4-sulfonylamino)-phenoxy]-2-(toluene-4-sulfonylamino)-benzoic acid methyl ester:

MS: [M−H]⁻=583.

NMR: ¹H (CDCl3): δ 10.35 (s, 1H), 7.73-7.68 (m, 3H), 7.63 (d, J=8.32 Hz, 2H), 7.53-7.46 (m, 2H), 7.28-7.21 (m, 3H), 7.10 (dd, J=3.00 Hz and J=9.08 Hz, 1H), 6.65 (ddd, J=9.08 Hz and J=2.76 Hz and J=1.48 Hz, 1H), 6.55-6.48 (m, 2H), 3.84 (s, 3H), 2.40 (s, 3H), 2.38 (s, 3H).

5-[5-fluoro-2-(toluene-4-sulfonylamino)-phenoxy]-2-(toluene-4-sulfonylamino)-benzoic acid methyl ester:

MS: [M−H]⁻=583.

NMR: ¹H (CDCl3): δ 10.38 (s, 1H), 7.72 (d, J=8.32 Hz, 2H), 7.63 (d, J=5.80 Hz and J=9.12 Hz, 1H), 7.60 (d, J=9.08 Hz, 1H), 7.55 (d, J=8.32 Hz, 2H), 7.28-7.24 (m, 4H), 7.14 (d, J=8.08 Hz, 2H), 6.82-6.74 (m, 2H), 6.60 (d, J=2.80 Hz and J=9.08 Hz, 1H), 6.20 (dd, J=2.80 Hz and J=9.12 Hz, 1H), 3.86 (s, 3H), 2.39 (s, 3H), 2.36 (s, 3H).

### Preparation of mixture of 5-(4-Amino-3-fluoro-phenoxy)-2-(toluene-4-sulfonylamino)-benzoic acid methyl ester and 5-(2-Amino-5-fluoro-phenoxy)-2-(toluene-4-sulfonyl-amino)-benzoic acid methyl ester

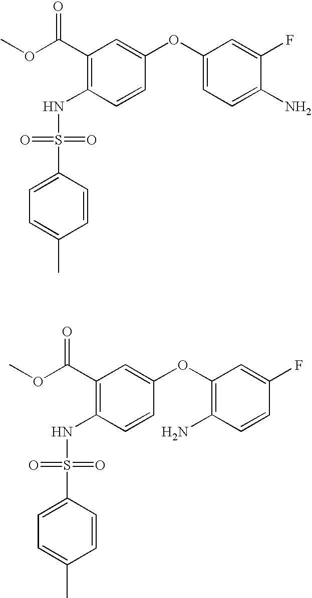

At RT, 10% Pd—C (177 mg) was added to a mixture of 5-(4-nitro-3-fluoro-phenoxy)-2-(toluene-4-sulfonylamino)-benzoic acid methyl ester and 5-(2-nitro-5-fluoro-phenoxy)-2-(toluene-4-sulfonylamino)-benzoic acid methyl ester (1.67 g, 3.63 mmol) in THF/MeOH: 1/1 (30 mL). The reaction was placed under 1 atmosphere of hydrogen and was stirred at RT for 16 hours. The reaction mixture was filtered over Kiezelguhr and evaporated to dryness in vacuo to afford the desired products as a light brown oil (1.55 g, 99%, purity (LC)>95%).

MS: [M−H]⁻=429.

### Preparation of mixture of 5-(4-nitro-3-fluoro-phenoxy)-2-(toluene-4-sulfonylamino)-benzoic acid methyl ester and 5-(2-nitro-5-fluoro-phenoxy)-2-(toluene-4-sulfonyl-amino)-benzoic acid methyl ester

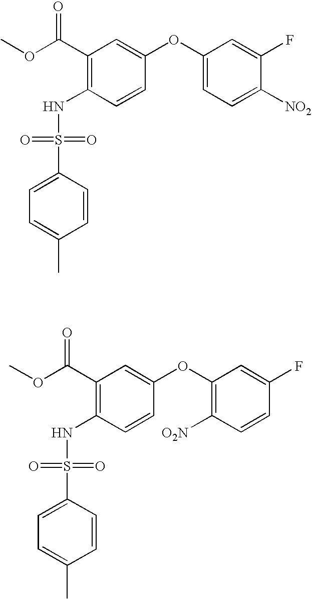

A mixture of methyl 5-hydroxy-2-[(4-methylphenyl)sulfonyl]aminobenzoate (1.50 g, 4.67 mmol), commercially available 2,4-difluoronitrobenzene (746 mg, 4.69 mmol), and potassium carbonate (1.61 g, 11.7 mmol) in DMF (30 mL) was stirred at 80° C. for 3 hours. The reaction was cooled to RT and diluted with EtOAc (150 mL). The reaction was washed with aq. 0.5 M KHSO₄ (200 mL), aq. sat. NaHCO₃ (150 mL), and brine (100 mL). The aqueous-layers were extracted once more with EtOAc (100 mL). The combined EtOAc-layers were dried (Na₂SO₄), filtered and evaporated to dryness in vacuo. The crude product was purified by flash column chromatography (EtOAc/heptane: 1/6 to 1/3) to obtain the a mixture of isomers as a yellow foam (1.67 g, 78%, purity (LC)>95%).

MS: [M−H]⁻=459.

### Compound 34

### Preparation of 5-(3-hydroxymethylene-4-[(4-methylphenvl)sulfonyl]aminophenoxy)-2-[(4-methylphenyl)sulfonyl]aminobenzoic acid

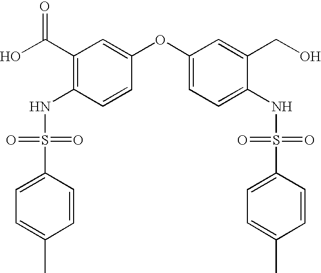

5-[3-(tert-Butyl-dimethyl-silanyloxymethyl)-4-(toluene-4-sulfonylamino)-phenoxy]-2-(toluene-4-sulfonylamino)-benzoic acid methyl ester (116 mg, 0.16 mmol) and lithium hydroxide (LiOHxH₂O) (34 mg, 0.82 mmol)) were dissolved in THF:H₂O (1:1)(10 mL) Reaction mixture was stirred at 70° C. for 5 h. Solution acidified using 1M HCl and the white solid filtrated off and dried. Product purified using preparative TLC (CH₂Cl₂: MeOH 9:1) (65 mg, 68%, purity (LC)=95.26%).

MS: [M−H]⁺=581.

NMR: ¹H (DMSO-d6, 300 MHz): δ 9.27 (s, 1H), 7.58 (d, J=8.1 Hz, 2H), 7.49 (d, J=8.1 Hz, 2H), 7.245-7.25 (m, 6H), 7.02 (dd, J=1.8 Hz and J=8.1 Hz, 1H), 6.9 (d, J=2.4 Hz, 1H), 6.74 (d, J=8.4 Hz, 1H), 6.65 (dd, J=2.4 Hz and J=8.4 Hz, 1H), 5.14 (m, 1H), 4.28 (d, J=4.2 Hz, 2H), 2.36 (s, 3H), 2.31 (s, 3H).

### Preparation of 5-[3-Hydroxymethyl-4-(toluene-4-sulfonylamino)-phenoxy]-2-(toluene-4-sulfonylamino)-benzoic acid methyl ester

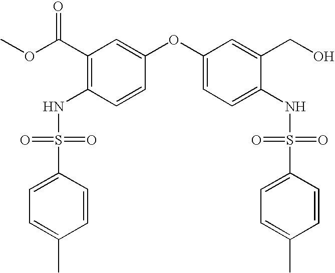

At RT, TBAF (127 mg, 0.403 mmol) was added to a solution of 5-[3-(tert-Butyl-dimethyl-silanyloxymethyl)-4-(toluene-4-sulfonylamino)-phenoxy]-2-(toluene-4-sulfonylamino)-benzoic acid methyl ester (151 mg, 0.213 mmol) in DMF (2 mL). The reaction was stirred at RT for 1 hour. The reaction mixture was diluted with EtOAc (30 mL) and washed with brine (3×50 mL). The aqueous-layers were extracted once more with EtOAc (1×30 mL). The combined EtOAc-extracts were dried (Na₂SO₄), filtered, and evaporated to dryness in vacuo. The crude product was purified by preparative TLC (EtOAc/heptane: 1/1) to obtain the desired product (116 mg, 91%, purity (LC)>95%).

MS: [M−H]⁻=595

NMR: ¹H (CDCl3): δ 7.60 (d, J=8.34 Hz, 2H), 7.54 (d, J=8.34 Hz, 2H), 7.42 (d, J=8.84 Hz, 1H), 7.39-7.33 (m, 4H), 7.30-7.23 (m, 2H), 6.96 (d, J=2.78 Hz, 1H), 6.83 (d, J=8.59 Hz, 1H), 6.77 (dd, J=2.78 Hz and J=8.84 Hz, 1H), 4.33 (s, 2H), 3.73 (s, 3H), 2.37 (s, 3H), 2.34 (s, 3H).

### Preparation of 5-[3-(tert-Butyl-dimethyl-silanyloxymethyl)-4-(toluene-4-sulfonylamino)-phenoxy]-2-(toluene-4-sulfonylamino)-benzoic acid methyl ester

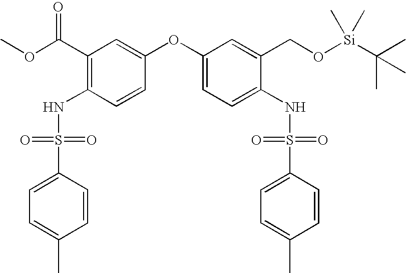

A solution of 5-[4-amino-3-(tert-butyl-dimethyl-silanyloxymethyl)-phenoxy]-2-(toluene-4-sulfonylamino)-benzoic acid methyl ester (164 mg, 0.295 mmol), tosyl chloride (87.8 mg, 0.460 mmol), and pyridine (51.4 mg, 0.650 mmol) in DCM (2 mL) was stirred at reflux for 22 hours. The reaction mixture was diluted with DCM (20 mL) and washed with aq. 0.5 N KHSO₄ (20 mL) and aq sat. NaHCO3 (20 mL). The aqueous-layers were extracted once more with DCM (20 mL). The combined DCM-layers were dried (Na₂SO₄), filtered, and evaporated to dryness in vacuo. The crude product was purified by using flash column chromatography (EtOAc/heptane: 1/8 to 1/3) to obtain the desired product (171 mg, 82%, purity (LC)>95%).

MS: [M−H]⁻=709.

### Preparation of 5-[4-Amino-3-(tert-butyl-dimethyl-silanyloxymethyl)-phenoxy]-2-(toluene-4-sulfonylamino)-benzoic acid methyl ester

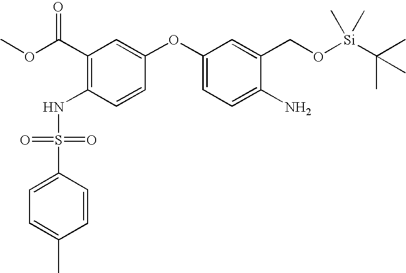

At RT, 10% Pd—C (27 mg) was added to a solution of 5-[4-nitro-3-(tert-butyl-dimethyl-silanyloxymethyl)-phenoxy]-2-(toluene-4-sulfonylamino)-benzoic acid methyl ester (217 mg, 0.370 mmol) in THF/MeOH: 1/1 (10 mL). The reaction mixture was placed under 1 atmosphere of hydrogen and stirred at RT for 18 hours. An additional amount of 10% Pd—C (30 mg) was added and the reaction was continued for 4 hours. The reaction mixture was filtered over Kiezelguhr and evaporated to dryness in vacuo. The crude product was purified by flash column chromatography (EtOAc/heptane: 1/8 to 1/4) to obtain the desired product as a colourless oil (164 mg, 80%, purity (LC)>95%). MS: [M−H]⁻=555.

### Preparation of 5-[4-nitro-3-(tert-butyl-dimethyl-silanyloxymethyl)-phenoxy]-2-(toluene-4-sulfonylamino)-benzoic acid methyl ester

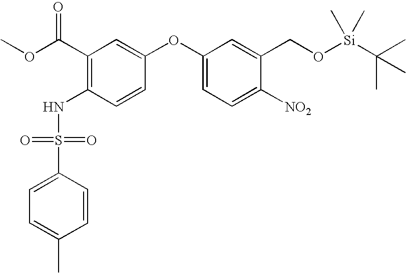

At RT, di-methyl-tert-butylsilyl chloride (106 mg, 0.703 mmol) was added to a solution of 5-[4-nitro-3-hydroxymethyl-phenoxy]-2-(toluene-4-sulfonylamino)-benzoic acid methyl ester (256 mg, 0.542 mmol) and imidazole (73.1 mg, 1.07 mmol) in DCM (5 mL). The reaction was warmed at reflux for 3 hours. An additional amount of di-methyl-tert-butylsilyl chloride (24 mg) was added and the reaction was continued for 1 hour. The reaction mixture was diluted with water (20 mL) and extracted with DCM (2×20 mL). The combined DCM-layers were dried (Na₂SO₄), filtered, and evaporated to dryness in vacuo. The crude product was purified by flash chromatography (EtOAc/heptane: 1/8 to 1/4) to afford the desired product as a white solid (218 mg, 69%, purity (LC) n.d.). The product was used as such in the following reactions.

### Preparation of 5-[4-nitro-3-hydroxymethyl-phenoxy]-2-(toluene-4-sulfonylamino)-benzoic acid methyl ester

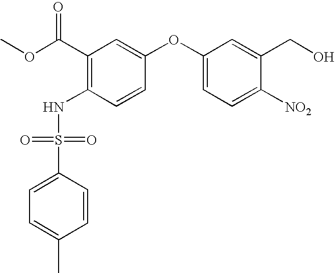

A mixture of methyl 5-hydroxy-2-[(4-methylphenyl)sulfonyl]aminobenzoate (445 mg, 1.38 mmol), 2-Hydroxymethylene-4-fluoronitrobenzene (237 mg, 1.38 mmol), and potassium carbonate (380 mg, 2.76 mmol) in DMF (5 mL) was stirred at 80° C. for 3 hours. The reaction was poured into aq. 0.5 N KHSO₄ (50 mL). The aqueous layer was extracted with EtOAc (2×30 mL). The EtOAc-extracts were washed with brine (2×50 mL). The combined EtOAc-extracts were dried (Na₂SO₄), filtered, and evaporated to dryness in vacuo. The crude product was purified by column chromatography (EtOAc/heptane: 1/10 to 2/3) to afford the desired product (264 mg, 40%, purity (LC)=80%).

MS: [M−H]⁻=471.

### Preparation of 2-Hydroxymethylene-4-fluoronitrobenzene

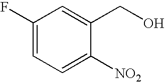

At 0° C. under N₂ atmosphere, 1 M borane-THF (11.4 mL, 11.4 mmol) was added gradually to solution of commercially available 5-fluoro-2-nitrobenzoic acid (700 mg, 3.78 mmol) in anhydrous THF (10 mL). The reaction mixture was warmed at 70° C. for 2 hours. The reaction was cooled to RT and a solution of aq. 1M NaOH (15 mL) was added dropwise. The reaction was evaporated to dryness in vacuo. The residue was dissolved in EtOAc (30 mL) and water (30 mL). The organic layer was isolated and washed with brine (20 mL). The aqueous-layers were extracted once more with EtOAc (20 mL). The combined EtOAc-extracts were dried (Na₂SO₄), filtered, and evaporated to dryness in vacuo to afford 2-hydromethylene-4-fluoronitrobenzene as a yellow solid (601 mg, 93%, purity (LC) n.d.).

NMR: ¹H (CDCl3): δ 8.21 (dd, J=5.08 Hz and J=9.12 Hz, 1H), 7.55 (dd, J=2.76 Hz and J=9.32 Hz, 1H), 7.13 (ddd, J=2.76 Hz and J=7.08 Hz and J=9.60 Hz, 1H), 5.05 (d, J=5.56 Hz, 2H), 2.43 (t, J=5.84 Hz, 1H).

### Compound 35

### Preparation of 5-[3-Chloro-4-(toluene-4-sulfonylamino)-phenoxy]-2-(toluene-4-sulfonylamino)-benzoic acid

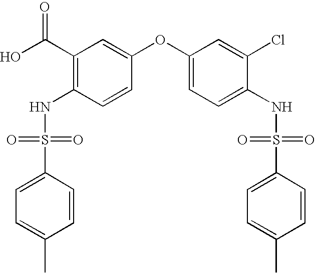

A solution of 5-[3-chloro-4-(toluene-4-sulfonylamino)-phenoxy]-2-(toluene-4-sulfonyl-amino)-benzoic acid methyl ester (68 mg, 0.113 mmol) and LiOH (29.6 mg, 0.705 mmol) in THF/water: 1/1 (10 mL) was heated at 70° C. for 5 hours. The reaction was cooled to RT and aq. 1 N HCl (30 mL) was added. The suspension was extracted with dichloromethane (3×30 mL). The combined DCM-extracts were dried (Na₂SO₄), filtered, and evaporated to dryness in vacuo. The residue was purified by preparative TLC (DCM/MeOH: 9/1). The product was triturated in EtOH/diisopropyl ether afford the desired product as a white solid (36.8 mg, 55%, purity (LC)=93%).

MS: [M−H]⁻=585.

NMR: ¹H (DMSO-d6): δ 9.81 (s, 1H), 7.61 (d, J=8.43 Hz, 2H), 7.54 (d, J=8.08 Hz, 2H), 7.38-7.34 (m, 4H), 7.27 (d, J=8.08 Hz, 2H), 7.13 (d, J=8.84 Hz, 1H), 6.97 (dd, J=3.04 Hz and J=8.84 Hz, 1H), 6.88 (d, J=2.76 Hz, 1H), 6.79 (d, J=2.76 Hz and J=8.84 Hz), 2.36 (s, 3H), 2.30 (s, 3H).

### Preparation of 5-[3-Chloro-4-(toluene-4-sulfonylamino)-phenoxy]-2-(toluene-4-sulfonylamino)-benzoic acid methyl ester

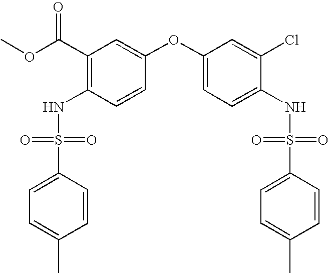

A solution of 5-(4-amino-3-chloro-phenoxy)-2-(toluene-4-sulfonylamino)-benzoic acid methyl ester (63 mg, 0.141 mmol), tosyl chloride (34.2 mg, 0.179 mmol), and pyridine (18.6 mg, 0.235 mmol) in CH₂Cl₂ (2 mL) was heated at reflux for 23 hours.Another batch of tosyl chloride (28.8 mg) and pyridine (35.2 mg) were added to the reaction mixture and the reaction was continued for 24 hours. The reaction was diluted with DCM (40 mL) and washed with aq. 0.5 N KHSO₄ (1×50 mL) and aq. saturated NaHCO3 (2×40 mL). The water layers were extracted once more with DCM (40 mL). The combined DCM-layers were dried (NaSO₄), filtered, and evaporated to dryness in vacuo. The crude product was purified by preparative TLC (EtOAc/heptane:1/2) to obtain the desired product as a white solid (68 mg, 81%, purity (LC)>93%).

MS: [M−H]⁻=600.

### Preparation of 5-(4-Amino-3-chloro-phenoxy)-2-(toluene-4-sulfonylamino)-benzoic acid methyl ester

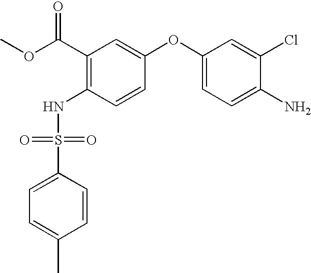

At RT, 10% Pd—C was added to a solution of 5-(3-chloro-4-nitro-phenoxy)-2-(toluene-4-sulfonylamino)-benzoic acid methyl ester (182 mg, 0.382 mmol) in THF/MeOH: 1/1 (10 mL). The reaction was placed under 1 atmosphere of hydrogen and stirred at RT for 3 hours. The reaction was filtered over Kiezelguhr and evaporated to dryness in. The crude product was purified by column chromatography to obtain the desired amine (67 mg, 39%, purity (LC)=93%).

MS: [M+H]⁺=447.

### Preparation of 5-(3-chloro-4-nitro-phenoxy)-2-(toluene-4-sulfonylamino)-benzoic acid methyl ester

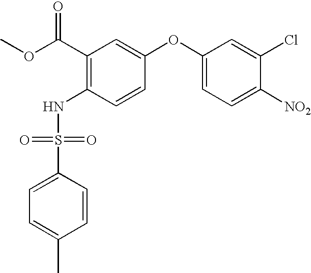

A mixture of methyl 5-hydroxy-2-[(4-methylphenyl)sulfonyl]aminobenzoate (354 mg mg, 1.07 mmol), 2-chloro-4-fluoronitrobenzene (201 mg, 1.14 mmol), and potassium carbonate (370 mg, 2.67 mmol) in DMF (10 mL) was stirred at 80° C. for 1 hour. The reaction was diluted with H₂O (50 mL) and extracted with EtOAc (2×50 mL). The EtOAc-extracts were washed once more with H2O (50 mL). The combined organic extracts were dried (Na₂SO₄), filtered, and evaporated to dryness in vacuo. The crude product was purified by column chromatography (EtOAc/heptane: 1/2) and then recrystallized from EtOAc/heptane to obtain the desired compound (182 mg, 36%, purity (LC) n.d.).

NMR: ¹H (300 Mhz, CDCl₃): δ 10.45 (s, 1H), 7.92 (d, J=9.00 Hz, 1H), 7.77-7.70 (m, 3H), 7.59 (d, J=2.70 Hz, 1H), 7.26-7.15 (m, 4H), 6.93 (d, J=2.70 Hz, 1H), 6.83 (dd, J=2.70 Hz and J=9.00 Hz, 1H), 3.86 (s, 3H), 2.39 (s, 3H).

MS: [M+H]⁺=447

### Preparation of 2-chloro-4-fluoronitrobenzene

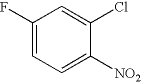

A 500 mL flask was charged with commercially available 1-chloro-3-fluorobenzene (20.0 g, 0.153 mol) and methane sulfonic acid (100 mL). The reaction was vigorously stirred and sodium nitrate (13.0 g, 0.153 mol) was added in small portions to the reaction mixture while the temperature was maintained below 30° C. using a water bath for external cooling. After addition of sodium nitrate the reaction was stirred at RT for 4 hours. The reaction mixture was poured into 500 mL ice-water and the aqueous layer was extracted with dichloromethane (3×250 mL). The organic extracts were washed once with saturated NaHCO₃/H₂O (500 mL). The combined extracts were dried (Na₂SO₄), filtered, and evaporated to dryness in vacuo. The crude product was distilled (bp 71° C., 2.3 torr) to obtain 2-chloro-4-fluoronitrobenzene as a clear oil (14.6 g, 54%, purity (GC)=84%).

### Compound 36

### Preparation of 5-[3-cyano-4-(toluene-4-sulfonylamino)-phenoxy]-2-(toluene-4-sulfonylamino)-benzoic acid

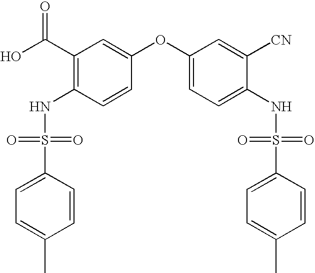

A solution of 5-[3-cyano-4-(toluene-4-sulfonylamino)-phenoxy]-2-(toluene-4-sulfonyl-amino)-benzoic acid methyl ester (37.0 mg, 0.063 mol) in aq. 0.1 M LiOH (2 mL) and THF (3 mL) was stirred at RT for 16 hours. An additional amount of aq. 0.1 M LiOH (2 mL) was added and the reaction was continued at 40° C. for 12 hours. The reaction was cooled to RT and aq. 1M HCl was added (10 mL). The suspension was extracted with DCM (3×10 mL). The combined DCM-layers were dried (Na₂SO₄), filtered, and evaporated to dryness in vacuo. The crude product was purified by preperative TLC (DCM/MeOH: 9/1+1% AcOH) to obtain the desired product (34.0 mg, 93%, purity (LC)>95%).

MS: [M−H]⁻=576.

NMR: ¹H (DMSO-d6): δ 7.61-7.56 (m, 4H), 7.30 (d, J=8.84 Hz, 1H), 7.28-7.22 (m, 3H), 7.17 (d, J=8.08 Hz, 2H), 7.07 (d, J=9.08 Hz, 1H), 6.85 (d, J=3.04 Hz, 1H), 6.81-6.74 (m, 2H), 2.30 (s, 3H), 2.29 (s, 3H).

### Preparation of 5-[3-cyano-4-(toluene-4-sulfonylamino)-phenoxy]-2-(toluene-4-sulfonylamino)-benzoic acid methyl ester

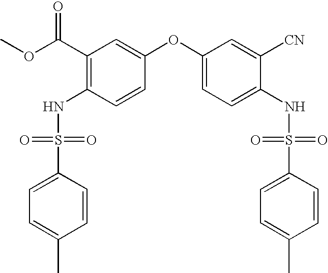

A suspension of 5-[3-carbamoyl-4-(toluene-4-sulfonylamino)-phenoxy]-2-(toluene-4-sulfonylamino)-benzoic acid methyl ester (83.6 mg, 0.141 mmol) and POCl₃ (3 mL) in anhydrous THF (2 mL) was stirred at RT for 72 hours and then at 40° C. for 24 hours. The reaction mixture was poured gradually into ice-cold aq. sat. NaHCO₃ (100 mL). The aqueous-layer was extracted with DCM (2×30 mL). The DCM-layers were washed with aq. sat. NaHCO₃ (1×30 mL). The water-layers were extracted with EtOAc (2×30 mL). The combined DCM and EtOAc-extracts were dried (Na₂SO₄), filtered and evaporated to dryness to obtain a yellow oil containing a white precipitate. The product was purified by flash column chromatography (EtOAc/heptane: 1/1) to obtain a yellow oil containing a precipitate. This product was triturated with ice-cold diisopropyl ether (2 mL) to afford the desired product as a solid (37 mg, 44%, purity (LC)>95%).

MS: [M−H]⁻=590.

### Preparation of 5-[3-carbamoyl-4-(toluene-4-sulfonylamino)-phenoxy]-2-(toluene-4-sulfonylamino)-benzoic acid methyl ester

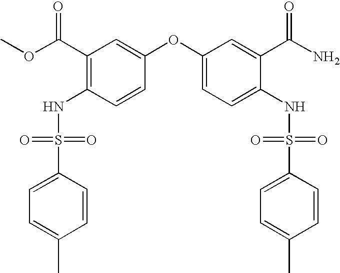

A suspension of methyl 5-[4-amino-3-(aminocarbonyl)phenoxy]-2-[(4-methylphenyl)-sulfonyl]aminobenzoate (106 mg, 0.233 mmol), tosyl chloride (65.4 mg, 0.343 mmol), and pyridine (45.0 mg, 0.569 mmol) in DCM (2 mL) and THF (2 mL) was stirred at RT for 72 hours. The suspension was diluted with DCM (100 mL) and washed with aq. 0.5 N KHSO₄ (1×50 mL) and aq. sat. NaHCO3 (1×50 mL). The water-layers were extracted once more with DCM (50 mL). The cloudy DCM layers were dried (Na₂SO₄), filtered and evaporated to dryness in vacuo. The crude product was purified by flash column chromatography (EtOAc/heptane: 1/4 to 2/1 then DCM/MeOH: 4/1) to obtain the desired product as a yellow solid (83.6 mg, 59%, purity (LC)>95%).

MS: [M−H]⁻=608

### Preparation of methyl 5-[4-amino-3-(aminocarbonyl)phenoxy]-2-[(4-methylphenyl)-sulfonyl]aminobenzoate

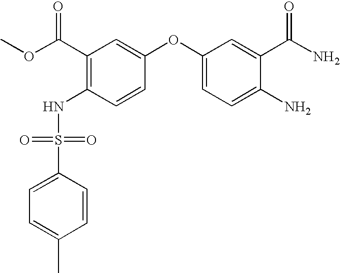

At RT and under N₂, a solution of 5-[3-cyano-4-nitro-phenoxy]-2-(toluene-4-sulfonyl-amino)-benzoic acid methyl ester (292 mg, 0.625 mmol) in THF/MeOH: 1/1 (10 mL) was added gradually to a suspension of iron (141 mg, 2.53 mmol) and ammonium chloride (131 mg, 2.45 mmol) in water (10 mL). The reaction was heated at 70° C. for 2 hours. The reaction was cooled to RT and filtered over kiezelguhr. Filtrate was extracted with EtOAc (2×30 mL). The combined EtOAc extracts were dried (Na₂SO₄), filtered and evaporated to dryness in vacuo. The crude product was purified by column chromatography (EtOAc/heptane: 1/3 to 2/1) to obtain methyl 5-[4-amino-3-(amino-carbonyl)phenoxy]-2-[(4-methylphenyl)sulfonyl]aminobenzoate (107 mg, 38%, purity (LC)>95%).

MS: [M+H]⁺=456

### Preparation of 5-[3-cyano-4-nitro-phenoxy]-2-(toluene-4-sulfonylamino)-benzoic acid methyl ester

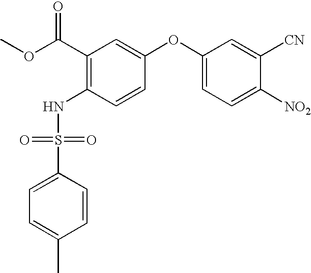

A mixture of methyl 5-hydroxy-2-[(4-methylphenyl)sulfonyl]aminobenzoate (252 mg, 0.785 mmol), 4-fluoro-2-cyanonitrobenzene (137 mg, 0.825 mmol), and potassium carbonate (271 mg, 1.96 mmol) in DMF (5 mL) was stirred at 80° C. for 2.5 hours. The reaction was diluted with EtOAc (40 mL) and washed with aq. 0.5 N KHSO₄ (1×40 mL) and aq. saturated NaHCO₃ (1×40 mL). The water layers were extracted once more with EtOAc (40 mL). The combined EtOAc-layers were dried (Na₂SO₄), filtered, and evaporated to dryness in vacuo. The crude product was purified by flash column chromatography (EtOAc/heptane: 1/3 to 1/1) to obtain the desired product as yellow solid (292 mg, 79%, purity (LC)=85%).

MS: [M−H]⁻=466

### Preparation of 4-fluoro-1-nitro-2-cyanobenzene

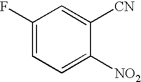

At RT, cyanuric chloride (1.04 g, 5.65 mmol) was added to a solution of 5-fluoro-2-nitrobenzamide (1.04 g, 5.65 mmol) in anhydrous THF (10 mL). The reaction was warmed at reflux for 23 hours. The reaction was diluted with DCM (100 mL) and washed with aq. sat. ammonium chloride (2×100 mL) and aq. sat. sodium bicarbonate (1×100 mL). The water-layers were extracted once more with DCM (100 mL). The combined DCM-layers were dried (Na₂SO₄), filtered, and evaporated to dryness in vacuo. The crude product was purified using column chromatography (EtOAc/heptane: 1/1) to afford 4-fluoro-1-nitro-2-cyanobenzene (315 mg, 34%, purity (LC)>95%)

MS: [M−H]⁻=165

### Preparation of 5-fluoro-2-nitrobenzamide

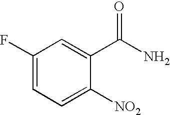

At 0° C. and under N₂, a solution of oxalylchloride (1.83 g, 14.4 mmol) in anhydrous THF (3 mL) was added gradually to a solution of commercially available 5-fluoro-2-nitrobenzoic acid (1.78 g, 9.62 mmol) and DMF (40 mg) in anhydrous THF (10 mL). The reaction was stirred at RT for 1.5 hours. The reaction mixture was evaporated to dryness in vacuo to afford 5-fluoro-2-nitro-1-benzenecarbonyl chloride as a yellow oil (2.13 g). At RT, a solution of 5-fluoro-2-nitro-1-benzenecarbonyl chloride (1.53 g) in anhydrous THF (9 mL) was added gradually to an ice-cold solution of 35% NH₄OH (30 mL). The reaction was stirred for 45 minutes and then 50 mL of dichloromethane was added to the reaction mixture. The organic layer was isolated and the water layer was extracted once more with dichloromethane (50 mL). The DCM-extracts were washed with aq. sat. NH₄Cl (1×50 mL). The combined organic extracts were dried (Na₂SO₄), filtered, and evaporated to dryness to obtain 5-fluoro-2-nitrobenzamide (1.04 g, 75%, purity (LC)>95%).

MS: [M+H]⁺=185

### Compound 37

### Preparation of 5-[3-Methoxy-4-(toluene-4-sulfonylamino)-phenoxy]-2-(toluene-4-sulfonylamino)-benzoic acid

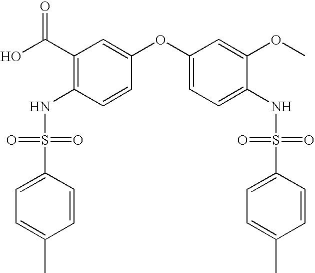

A solution of 5-[3-methoxy-4-(toluene-4-sulfonylamino)-phenoxy]-2-(toluene-4-sulfonylamino)-benzoic acid methyl ester (180 mg, 0.302 mmol) and LiOH (69 mg, 1.69 mmol) in THF/water: 1/1 (10 mL) was heated at 70° C. for 4 hours. The reaction was cooled to RT and aq. 1 N HCl (30 mL) was added. The resulting suspension was extracted with dichloromethane (2×30 mL). The combined DCM-extracts were dried (Na₂SO₄), filtered, and evaporated to dryness in vacuo. The crude product was purified by column chromatography (DCM/MeOH: 9/1) to obtain the desired product as a white solid (147 mg, 84%, purity (LC)>95%).

MS: [M−H]⁻=581.

NMR: ¹H (DMSO-d6): δ 10.87 (s, 1H), 9.37 (s, 1H), 7.66 (d, J=8.08 Hz, 2H), 7.54-7.50 (m, 3H), 7.37-7.30 (m, 5H), 7.25 (dd, J=3.04 Hz and 8.84 Hz, 1H), 7.14 (d, J=8.56 Hz, 1H), 6.59 (d, J=2.52 Hz, 1H), 3.36 (s, 3H), 6.43 (dd, J=2.52 Hz and J=8.56, 1H), 2.34 (s, 6H).

### Preparation of 5-[3-Methoxy-4-(toluene-4-sulfonylamino)-phenoxy]-2-(toluene-4-sulfonylamino)-benzoic acid methyl ester

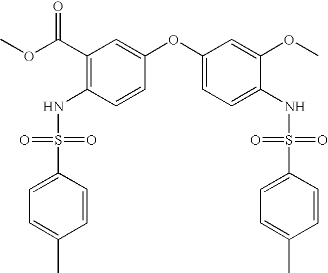

A solution of 5-(4-amino-3-methoxy-phenoxy)-2-(toluene-4-sulfonylamino)-benzoic acid methyl ester (191 mg, 0.432 mmol), tosyl chloride (127 mg, 0.666 mmol), and pyridine (72 mg, 0.910 mmol) in CH₂Cl₂ (4 mL) was heated at reflux for 25 hours. The reaction was diluted with DCM (40 mL) and washed with 0.5 N KHSO₄/water (1×50 mL) and saturated NaHCO₃/water (1×40 mL). The water layers were extracted once more with DCM (40 mL). The combined DCM-layers were dried (Na₂SO₄), filtered, and evaporated to dryness in vacuo. The crude product was purified by column chromatography (EtOAc/heptane: 1/2 to 1/1) to afford the desired product (195 mg, 75%, purity (LC)>95%).

MS: [M−1]⁻=595

### Preparation of 5-(4-Amino-3-methoxy-phenoxy)-2-(toluene-4-sulfonylamino)-benzoic acid methyl ester

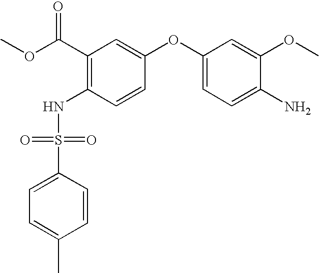

A suspension of 5-(4-nitro-3-methoxy-phenoxy)-2-(toluene-4-sulfonylamino)-benzoic acid methyl ester (188 mg, 0.398 mmol) and 10% Pd—C (20 mg) in MeOH/THF: 1/1 was placed under 1 atmosphere of hydrogen and stirred at RT for 3 hours. The reaction was filtered over Kiezelguhr and the filtrate was evaporated to dryness in vacuo to obtain 5-(4-amino-3-methoxy-phenoxy)-2-(toluene-4-sulfonylamino)-benzoic acid methyl ester as a brown solid (174 mg, 99%, purity (LC)>90%).

MS: [M−1]⁻=441

### Preparation of 5-(4-nitro-3-methoxy-phenoxy)-2-(toluene-4-sulfonylamino)-benzoic acid methyl ester

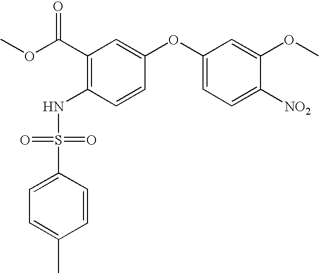

A mixture of methyl 5-hydroxy-2-[(4-methylphenyl)sulfonyl]aminobenzoate (277 mg mg, 0.861 mmol), 4-fluoro-2-methoxynitrobenzene (199 mg, 0.905 mmol), and potassium carbonate (297 mg, 2.15 mmol) in DMF (10 mL) was stirred at 80° C. for 3 hours. The reaction was diluted with EtOAc (40 mL) and washed with aq. 0.5 N KHSO₄ (1×40 mL) and aq. saturated NaHCO3 (2×40 mL). The water layers were extracted with EtOAc (30 mL). The combined EtOAc-layers were dried (Na₂SO₄), filtered, and evaporated to dryness in vacuo. The crude product was purified by column chromatography (EtOAc/heptane: 1/2). The product from the column was triturated in EtOH to obtain 5-(4-nitro-3-methoxy-phenoxy)-2-(toluene-4-sulfonylamino)-benzoic acid methyl ester (185 mg, 45%, purity (LC)=80%).

MS: [M−H]⁻=471

### Preparation of 4-fluoro-2-methoxynitrobenzene

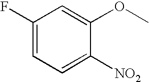

A mixture of commercially available 5-fluoro-2-nitrophenol (1.00 g, 6.37 mmol), MeI (1.36 g, 9.58 mmol), and potassium carbonate (1.32 g, 9.55 mmol) in DMF (10 mL) was heated at 140° C. for 23 hours. The reaction was diluted with aq. 0.5 N NaOH (50 mL) and extracted with EtOAc (2×50 mL). The EtOAc-extracts were washed once more with aq. 0.5 N NaOH (50 mL). The combined organic layers were dried (Na₂SO₄), filtered, and evaporated to dryness in vacuo. The crude product was purified by flash column chromatography (EtOAc/heptane: 1/1) to obtain 4-fluoro-2-methoxy-nitrobenzene (416 mg, 38%, purity (GC)>95%).

MS: [M]⁺=171.

### Compound 38

### Preparation of 5-[3-Ethoxy-4-(toluene-4-sulfonylamino)-phenoxy]-2-(toluene-4-sulfonylamino)-benzoic acid

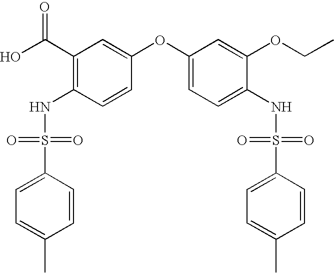

5-[3-Ethoxy-4-(toluene-4-sulfonylamino)-phenoxy]-2-(toluene-4-sulfonylamino)-benzoic acid methyl ester (60 mg, 0.098 mmol) was dissolved in a 1:1 mixture of water/THF (10 ml). Added LiOH monohydrate (21 mg, 0.491 mmol) and stirred at 50° C. for 5 hours. Distilled the THF off. Acidified with 2N HCl. The formed precipitate was filtered off and washed with demi-water. Dried in vacuo at 40° C. overnight. Purification by a preparative plate (2 mm layer thickness), eluens CH₂Cl₂:MeOH 9:1 (plus a few drops of concentrated HOAc). Scraped the most UV intense band of the plate. Removed the product from silica gel by stirring up in CH₂Cl₂ :MeOH 9:1 (+a few drops of concentrated HOAc). Filtered off, washed and concentrated in vacuo. Stripped with toluene and CH₂Cl₂. Gave 48 mg (y: 80%) product.

LC: >95%

MS: [M−H]⁺=595

NMR: ¹H (400 MHz, dmso-d6): δ9.28 (s, 1H), 7.65 (d, J=8.32 Hz, 2H), 7.52-7.48 (m, 3H), 7.35-7.29 (m, 5H), 7.21-7.17 (m, 2H), 6.54 (d, J=2.52 Hz, 1H), 6.41 (dd, J=2.8 Hz and J=8.84 Hz), 3.60 (q, J=7.08, 2H), 2.34 (s, 6H), 1.01 (t, J=6.84 Hz, 3H)

### Preparation of 5-[3-Ethoxy-4-(toluene-4-sulfonylamino)-phenoxy]-2-(toluene-4-sulfonylamino)-benzoic acid methyl ester

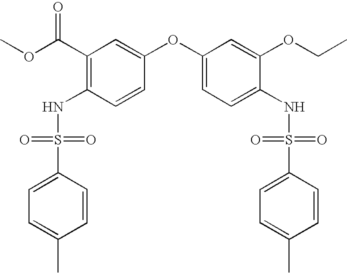

5-(4-Amino-3-ethoxy-phenoxy)-2-(toluene-4-sulfonylamino)-benzoic acid methyl ester (100 mg, 0.219 mmol) was dissolved in CH₂Cl₂ (10 ml) under Nitrogen. Added pyridine (36 μl, 0.438 mmol) and p-toluene-sulfonyl chloride (46 mg, 0.241 mmol).

Stirred at room temperature for 18 hours. Added CH₂Cl₂ and washed with 0.5N HCl (25 ml), 0.5N NaOH (25 ml) and aq. sat. NaCl (25 ml). Dried over Na₂SO₄, filtered off and concentrated in vacuo.

Purification by a preparative plate (2 mm layer thickness), eluens Heptane:EtOAc 1:1. Scraped the most UV intense band of the plate. Removed the product from silica gel by stirring up in CH₂Cl₂:MeOH 9:1. Filtered off, washed and concentrated in vacuo. Stripped with CH₂Cl₂. Gave 60 mg (y: 45%) product.

LC: 90%

MS: [M−H]⁺=609

### Preparation of 5-(4-Amino-3-ethoxy-phenoxy)-2-(toluene-4-sulfonylamino)-benzoic acid methyl ester

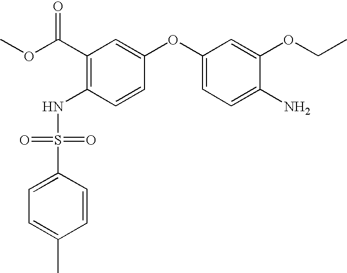

Dissolved NH₄Cl (82 mg, 1.54 mmol) in water (2 ml). Added Fe (86 mg, 1.54 mmol) followed by a suspension of 5-(3-Ethoxy-4-nitro-phenoxy)-2-(toluene-4-sulfonyl-amino)-benzoic acid methyl ester (150 mg, 0.308 mmol) in a mixture of 1:1 MeOH/THF (10 ml). Heated to 70° C. under Nitrogen for 4 hours. Cooled to room temperature. Filtered off over Kieselguhr, washed with EtOAc. The filtrate was washed with aq. sat. NaHCO₃ and aq. sat. NaCl. Dried over Na₂SO₄, filtered off and concentrated in vacuo.

Purification by a preparative plate (2 mm layer thickness), eluens Heptane:EtOAc 1:1. Scraped the most UV intense band of the plate. Removed the product from silica gel by stirring up in CH₂Cl₂ :MeOH 9:1. Filtered off, washed and concentrated in vacuo. Stripped with CH₂Cl₂. Gave 100 mg (y: 71%) product.

Purity determined by TLC.

### Preparation of 5-(3-Ethoxy-4-nitro-phenoxy)-2-(toluene-4-sulfonylamino)-benzoic acid methyl ester

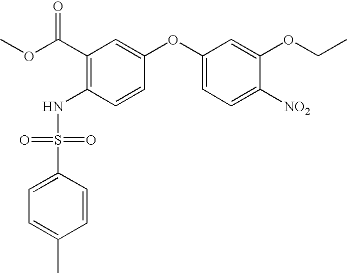

5-Hydroxy-2-(toluene-4-sulfonylamino)-benzoic acid methyl ester (200 mg, 0.622 mmol) was dissolved in dry DMF (5 ml) under Nitrogen. Added K₂CO₃ (215 mg, 1.56 mmol) and 2-Ethoxy-4-fluoro-1-nitro-benzene (121 mg, 0.653 mmol). Heated to 80° C. for 3 hours. Cooled to room temperature. Added EtOAc, washed with 0.5 M KHSO₄ (2×25 ml), aq. sat. NH₄Cl (25 ml), 0.5 M NaOH (2×25 ml) and aq. sat. NaCl (25 ml). Dried over Na₂SO₄, filtered off and concentrated in vacuo. Stripped with CH₂Cl₂. Stirred up in EtOH. Gave crystal formation. Filtered off and washed with EtOH, dried on filter and on vacuo line. Gave an off white solid 150 mg (y:50%).

LC: >95%

MS: [M+H]⁺=487

### Preparation of 2-Ethoxy-4-fluoro-1-nitro-benzene

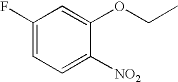

Dissolved the commercially available 5-fluoro-2-nitrophenol (250 mg, 1.59 mmol) in 2-butanone (10 ml) under Nitrogen. Added K₂CO₃ (659 mg, 4.77 mmol) and iodo-ethane (260 mg, 1.67 mmol). Stirred at 80° C. for 18 hours. Cooled, filtered off and washed with 2-butanone. The filtrate was concentrated in vacuo. Redissolved in EtOAc and washed with 0.5N NaOH (2×25 ml) and aq. sat. NaCl (25 ml). Dried over Na₂SO₄, filtered off and concentrated in vacuo. Gave 251 mg (85%) yellow liquid.

GC: >95%

MS: [M]⁺=185

### Compound 39

### Preparation of 5-[3-Propoxy-4-(toluene-4-sulfonylamino)-phenoxy]-2-(toluene-4-sulfonylamino)-benzoic acid

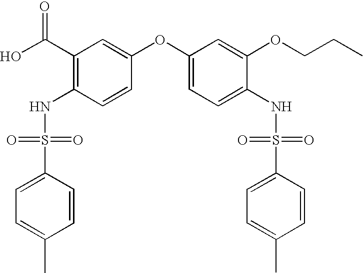

A mixture of 5-[3-Propoxy-4-(toluene-4-sulfonylamino)-phenoxy]-2-(toluene-4-sulfonylamino)-benzoic acid methyl ester (150 mg, 0.24 mmol), LiOH (40 mg, 0.96 mmol), water (2 mL) and THF (2 mL) was stirred for 16 hours at room temperature. The reaction was acidified to pH=1 with aq. 2N HCl and the THF was evaporated under reduced pressure. The suspension was extracted with DCM (2×50 mL) and the extracts were dried over anhydrous sodium sulfate. Concentration of the organic solution afforded the desired compound (141 mg, 96%, purity (LC)>95%).

MS: [M−H]⁻=637

NMR: ¹H (DMSO-d6): δ 10.91 (bs, 1H), 9.27 (s, 1H), 7.66 (d, J=8.08 Hz, 2H), 7.52-7.48 (m, 3H), 7.36 (d, J=8.08 Hz, 2H), 7.33 (d, J=3.04 Hz, 1H), 7.30 (d, J=8.08 Hz, 2H), 7.24 (dd, J=3.04 Hz and 8.84 Hz, 1H), 7.19 (d, J=8.56 Hz, 1H) 6.579 (d, J=2.52 Hz, 1H), 6.43 (dd, J=2.28 Hz and J=8.56 Hz, 1H), 3.51 (t, J=6.56 Hz, 2H), 2.34 (s, 3H), 2.34 (s, 3H), 1.46-1.37 (m, 2H), 0.79 (t, J=7.46 Hz, 3H)

### Preparation of 5-[3-Propoxy-4-(toluene-4-sulfonylamino)-phenoxy]-2-(toluene-4-sulfonylamino)-benzoic acid methyl ester

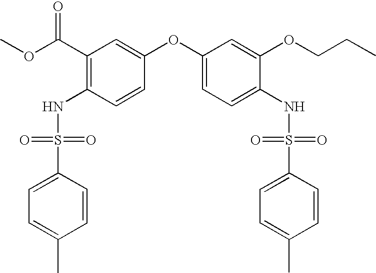

A solution of 5-(4-Amino-3-propoxy-phenoxy)-2-(toluene-4-sulfonylamino)-benzoic acid methyl ester (410 mg, 0.87 mmol), p-toluenesulfonylchloride (180 mg, 0.96 mmol) and pyridine (140 mg, 1.74 mmol) in dichloromethane (5 mL) was stirred for 72 hours at room temperature. The reaction was diluted with dichloromethane (50 mL) and washed with aqueous 0.5N potassium hydrogen sulfate (30 mL), aq. saturated sodium hydrogen carbonate (30 mL) and dried over anhydrous sodium sulfate. After concentration under reduced pressure, the residue was triturated in boiling DIPE containing 5% methanol. After cooling to 0° C., the crystals were filtered and dried in vacuo for 16 hours (295 mg, 54%, purity (LC)>95%).

MS: [M−H]⁻=623

NMR: ¹H (DMSO-d6): δ 10.05 (bs, 1H), 9.31 (bs, 1H), 7.61 (d, J=8.08 Hz, 2H), 7.51 (d, J=8.36 Hz, 2H), 7.41 (d, J=8.61 Hz, 1H), 7.35 (d, J=8.08 Hz, 1H), 7.30 (d, J=8.08 Hz, 1H), 7.26-7.20 (m, 2H), 7.20 (d, J=8.60 Hz, 1H) 6.59 (d, J=2.76 Hz, 1H), 6.44 (dd, J=2.25 Hz and J=8.60 Hz, 1H), 3.72 (s, 3H), 2.41 (s, 3H), 3.53 (t, J=6.56 Hz, 2H), 2.35 (s, 3H), 2.34 (s, 3H), 1.48-1.39 (m, 3H), 0.80 (t, J=7.44 Hz, 3H)

### Preparation of 5-(4-Amino-3-propoxy-phenoxy)-2-(toluene-4-sulfonylamino)-benzoic acid methyl ester

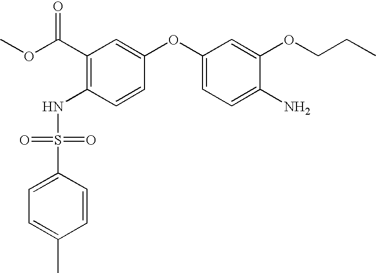

A suspension of 5-(4-nitro-3-propoxy-phenoxy)-2-(toluene-4-sulfonylamino)-benzoic acid methyl ester (455 mg, 0.91 mmol) and 10% palladium on activated carbon (48 mg, 0.045 mmol) in tetrahydrofuran (5 mL) and methanol (5 mL) was stirred at room temperature under 1 atmosphere of hydrogen. After 4 hours the reaction was filtered over Kieselguhr and evaporated under reduced pressure to afford the desired product (410 mg, 96%, purity n.d.).

### Preparation of 5-(4-nitro-3-propoxy-phenoxy)-2-(toluene-4-sulfonylamino)-benzoic acid methyl ester

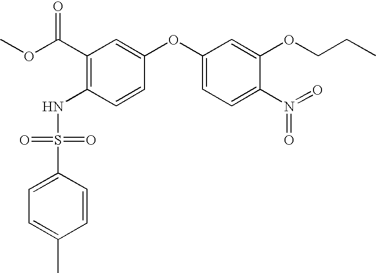

A suspension of 5-hydroxy-2-(toluene-4-sulfonylamino)-benzoic acid methyl ester (400 mg, 1.24 mmol), 4-fluoro-1-nitro-2-propoxy-benzene (227 mg, 1.24 mmol) and K₂CO₃ (430 mg, 3.1 mmol) in DMF (20 mL) was stirred at 80° C. After 16 hour the reaction was diluted with EtOAc (100) and washed with aq. 0.5N KHSO₄ (2×100 mL), aq. sat. NH₄Cl (100 mL), and aq. 0.5N NaOH (2×100 mL).The extracts were dried with brine and anh. Na₂SO₄ and evaporated under reduced pressure.The oily residue was stirred in ethanol (60 mL) for 0.5 hours and precipitation of a solid occurred. The suspension was stirred 0.5 hours at 0° C. and filtered. The solid was dried in vacuo at 40° C. for 16 hours to afford the desired product (430 mg, 75%, purity (LC) 94%).

MS: [M+H]⁺=501

### Preparation of 4-Fluoro-1-nitro-2-propoxy-benzene

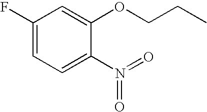

Dissolved commercially available 5-fluoro-2-nitrophenol (250 mg, 1.59 mmol) in 2 butanone (10 ml). Added K₂CO₃ (659 mg, 4.77 mmol) under Nitrogen. Added 1 iodo-propane (279 mg, 1.67 mmol) as a solution in 2-butanone (2 ml). Stirred at 80° C. for 18 hours. Cooled to room temperature, filtered off and washed with 2-butanone. The filtrate was concentrated in vacuo. Redissolved in EtOAc and washed with 0.5N NaOH (2×25 ml) and aq. sat. NaCl (25 m1).Dried over Na₂SO₄, filtered off and concentrated in vacuo. Gave 224 mg (y: 71%) yellow liquid

GC: >95%

MS: [M]⁺=199/157

### Compound 40

### Preparation of 2-(toluene-4-sulfonylamino)-5-[4-(toluene-4-sulfonylamino)-3-vinyl-phenoxy]-benzoic acid

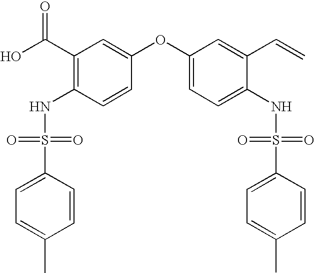

A mixture of 2-(toluene-4-sulfonylamino)-5-[4-(toluene-4-sulfonylamino)-3-vinyl-phenoxy]-benzoic acid methyl ester (150 mg, 0.253 mmol), LiOH (42 mg, 1.012 mmol), water (2 mL) and THF (2 mL) was stirred for 2 hours at 60° C. The reaction was acidified to pH=1 with aq. 1N HCl and the THF was evaporated under reduced pressure. The suspension was extracted with DCM (2×50 mL) and the extracts were dried over anhydrous sodium sulfate. Concentration of the organic solution afforded the desired compound. The solid was dried in vacuo for 16 hours (29 mg, 52%, purity (LC)>95%).

MS: [M−H]⁻=577

NMR: ¹H (DMSO-d6): δ 10.92 (bs, 1H), 9.64 (s, 1H), 7.66 (d, J=8.32 Hz, 2H), 7.53 (t, J=8.84 Hz, 1H), 7.48 (d, J=8.32 Hz, 2H), 7.36-7.32 (m, 5H), 7.27 (dd, J=3.04 Hz and 8.84 Hz, 1H), 7.19 (d, J=2.76 Hz, 1H), 6.86 (d, J=8.56 Hz, 1H), 6.79 (dd, J=2.52 and 8.56 Hz, 2H), 6.75 (dd, J=11.12 Hz and 17.68 Hz, 1H), 5.57 (d, J=17.44 Hz, 1H), 5.11 (d, J=11.36 Hz, 1H), 2.36 (s, 3H), 2.34 (s, 3H), 1.89 (s, 3H)

### Preparation of 2-(toluene-4-sulfonylamino)-5-[4-(toluene-4-sulfonylamino)-3-vinyl-phenoxy]-benzoic acid methyl ester

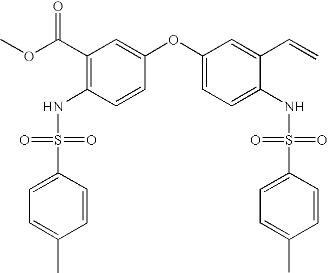

A solution of 5-(4-amino-3-vinyl-phenoxy)-2-(toluene-4-sulfonylamino)-benzoic acid methyl ester (229 mg, 0.52 mmol), p-toluenesulfonylchloride (110 mg, 0.57 mmol) and pyridine (82 mg, 1.04 mmol) in dichloromethane (5 mL) was stirred for 72 hours at room temperature and 24 hours at 40° C. The reaction was diluted with dichloromethane (50 mL) and washed with aqueous 0.5N potassium hydrogen sulfate (30 mL), saturated sodium hydrogen carbonate (30 mL) and dried over anhydrous sodium sulfate. After concentration under reduced pressure the residue was purified by column chromato-graphy (SiO₂) on elution with heptane/EtOAc 4:1 to obtain the desired product (150 mg, 49%, purity (LC)>95%).

MS: [M−H]⁻=491

### Preparation of 5-(4-amino-3-vinyl-phenoxy)-2-(toluene-4-sulfonylamino)-benzoic acid methyl ester

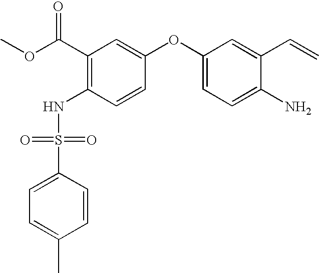

To a solution of ammonium chloride (131 mg, 2.45 mmol) in water (4 mL) was 325 mesh iron added (137 mg, 2.45 mmol) and the resulting suspension was stirred 10 minutes. Subsequently, a solution of 5-(4-nitro-3-vinyl-phenoxy)-2-(toluene-4-sulfonylamino)-benzoic acid methyl ester (230 mg, 0.49 mmol) in methanol (2 mL) and tetrahydrofuran (2 mL) was added and the reaction mixture was heated to 70° C. After 2 hours the reaction mixture was filtered over Kieselguhr and the filtrate was concentrated under reduced pressure to remove the tetrahydrofuran and methanol. The aqueous residue was redissolved in ethyl acetate (80 mL) and the organic solution was washed with aqueous saturated sodium hydrogen carbonate (60 mL) and brine (60 mL). The organic layer was dried over anhydrous sodium sulfate and evaporated under reduced pressure to obtain the product (128 mg, 60%, purity n.d.).

All aqueous layers were extracted back with dichloromethane and the Kieselguhr filter residue was rinsed with dichloromethane. The extracts were combined and dried over anhydrous sodium sulfate. Concentration under reduced pressure afforded a second batch of the desired product (85 mg, 39%, purity n.d.).

### Compound 41

### Preparation of 5-[3-Ethyl-4-(toluene-4-sulfonylamino)-phenoxy]-2-(toluene-4-sulfonylamino)-benzoic acid

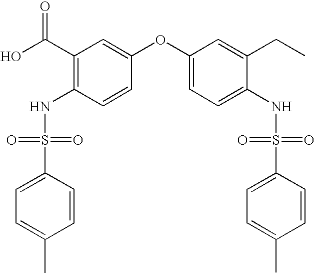

A mixture of 5-[3-Ethyl-4-(toluene-4-sulfonylamino)-phenoxy]-2-(toluene-4-sulfonyl-amino)-benzoic acid methyl ester (229 mg, 0.384 mmol) in aq. 0.5 M LiOH (5 mL) and THF (5 mL) was stirred at 60° C. for 3 hours. The reaction was cooled to RT and THF was evaporated in vacuo. The residue was acidified with aq. 1 M HCl (5 mL) and then diluted with water (25 mL). The aqueous suspension was extracted with DCM (2×20 mL). The combined DCM-extracts were dried (Na₂SO₄), filtered, and evaporated to dryness to obtain the desired product as a light red/white foam (197 mg, 88%, purity (LC)>95%).

NMR: ¹H (DMSO-d6): δ 10.81 (bs, 1H), 9.47 (s, 1H), 7.66 (d, J=8.08 Hz, 2H), 7.55-7.50 (m, 3H), 7.37-7.32 (m, 5H), 7.26 (dd, J=3.00 Hz and J=9.08 Hz, 1H), 6.83-6.79 (m, 2H), 6.67 (dd, J=3.00 Hz and J=8.56 Hz, 1H), 2.41 (q, J=7.56 Hz, 2H), 2.37 (s, 3H), 2.34 (s, 3H), 0.89 (t, J=7.56 Hz, 3H).

MS: [M−H]⁻=579

### Preparation of 5-[3-Ethyl-4-(toluene-4-sulfonylamino)-phenoxy]-2-(toluene-4-sulfonyl-amino)-benzoic acid methyl ester

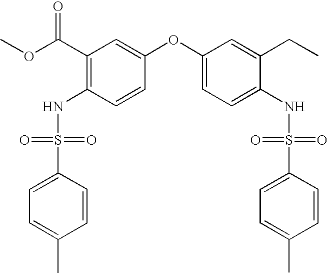

A solution of 5-(4-amino-3-ethyl-phenoxy)-2-(toluene-4-sulfonylamino)-benzoic acid methyl ester (232 mg, 0.527 mmol), tosyl chloride (151 mg, 0.790 mmol), and pyridine (83 mg, 1.05 mmol) in CH₂Cl₂ (5 mL) was stirred at 40° C. for 16 hours. The reaction was diluted with DCM (10 mL) and washed with aq. 0.5 N KHSO₄ (10 mL) and aq. sat. NaHCO₃ (10 mL). The aqueous-layers were extracted with DCM (1×10 mL). The combined DCM-layers were dried (Na₂SO₄), filtered and evaporated to dryness in vacuo. The crude product was purified by flash column chromatography (EtOAc/heptane: 1/4) to afford the desired product as an off-white foam (265 mg, 85%, purity (LC)>95%).

NMR: ¹H (CDCl₃): δ 10.30 (s, 1H), 7.72-7.66 (m, 3H), 7.60 (d, J=8.32 Hz, 2H), 7.51 (d, J=2.80 Hz, 1H), 7.27-7.21 (m, 3H), 7.15 (d, J=8.84 Hz, 1H), 7.10 (dd, J=2.80 Hz and J=9.12 Hz, 1H), 6.71 (d, J=2.76 Hz, 1H), 6.63 (dd, J=2.76 Hz and J=8.84 Hz, 1H), 6.18 (s, 1H), 3.82 (s, 3H), 2.41 (s, 3H), 2.38 (s, 3H), 2.32 (q, J=7.60 Hz, 2H), 0.99 (t, J=7.60 Hz, 3H).

MS: [M−H]⁻=593

### Preparation of 5-(4-Amino-3-ethyl-phenoxy)-2-(toluene-4-sulfonylamino)-benzoic acid methyl ester

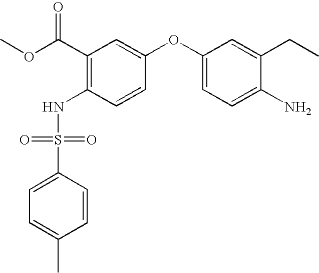

At RT, 10% Pd—C (48 mg) was added to a solution of 5-(4-nitro-3-vinyl-phenoxy)-2-(toluene-4-sulfonylamino)-benzoic acid methyl ester (247 mg, 0.527 mmol) in THF/MeOH: 1/1 (5 mL). The reaction was placed under 1 atmosphere of hydrogen and stirred at RT for 4 hours. The reaction was filtered over Kiezelguhr. The residue was rinsed with EtOH (2×2 mL) and THF (2×2 mL). The filtrate was evaporated to dryness to afford 5-(4-Amino-3-ethyl-phenoxy)-2-(toluene-4-sulfonylamino)-benzoic acid methyl ester as a yellow foam (232 mg, 100%, purity (LC)>95%).

MS: [M−H]⁻=439.

### Preparation of 5-(4-nitro-3-vinyl-phenoxy)-2-(toluene-4-sulfonylamino)-benzoic acid methyl ester

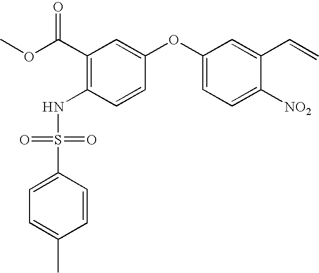

A mixture of methyl 5-hydroxy-2-[(4-methylphenyl)sulfonyl]aminobenzoate (530 mg mg, 1.65 mmol), 4-fluoro-1-nitro-2-vinylbenzene (251 mg, 1.51 mmol), and potassium carbonate (440 mg, 3.18 mmol) in DMF (10 mL) was stirred at 80° C. for 4 hours. The reaction mixture was cooled to RT, diluted with brine (100 mL) and extracted with EtOAc (3×100 mL). The EtOAc-extracts were washed with brine (2×50 mL). The combined EtOAc-layers were dried (Na₂SO₄), filtered, and evaporated to dryness in vacuo. The product was subjected to flash column chromatography (EtOAc/heptane: 1/12 to 1/4) to obtain the desired product as a white solid (188 mg, 24%, purity (LC) n.d.). A second crop of product was obtained by combining impure fractions from the column and purification by crystallization from EtOH to another batch of the desired product as small white crystals (303 mg, 38%, purity (LC)=85%).

NMR: ¹H (CDCl₃): δ 10.47 (s, 1H), 8.00 (d, J=9.08 Hz, 1H), 7.79-7.73 (m, 3H), 7.62 (d, J=3.04 Hz, 1H), 7.28-7.19 (m, 4H), 7.04 (d, J=2.76 Hz, 1H), 6.82 (dd, J=2.76 Hz and J=9.08 Hz, 1H), 5.60 (dd, J=0.76 Hz and J=17.2 Hz, 1H), 5.47 (dd, J=0.76 Hz and J=10.8 Hz, 1H), 3.86 (s, 3H), 2.40 (s, 3H).

MS: [M−H]⁻=467.

### Preparation of 4-fluoro-1-nitro-2-vinylbenzene

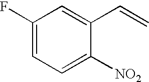

At RT and under Argon, Pd(PPh3)4 (67 mg, 0.058 mmol) was added to a solution of 2-bromo-4-fluoro-1-nitrobenzene (391 mg, 1.77 mmol) and tributyl(vinyl)tin (620 mg, 1.96 mmol) in toluene (degassed by purging, 10 mL). The reaction was stirred at reflux for 7 hours and then at RT for 60 hours. The reaction mixture was evaporated to dryness in vacuo. The residue was dissolved in CH₂Cl₂ (100 mL) and washed with aq. sat. NaHCO₃ (50 mL). The aqueous-layer was extracted once more with CH₂Cl₂ (50 mL). The DCM-extracts were dried (Na₂SO₄), filtered, and evaporated to dryness in vacuo. The crude product was purified by column chromatography (heptane to EtOAc/heptane: 1/4) to obtain 4-fluoro-1-nitro-2-vinylbenzene as a yellow oil (266 mg, 89%, purity (LC) n.d.)

NMR: ¹H (CDCl₃): δ 8.04 (dd, J=5.04 Hz and J=8.04 Hz, 1H), 7.29 (dd, J=2.52 Hz and J=9.08 Hz, 1H), 7.22 (ddd, J=1.00 Hz and J=10.8 Hz and J=17.2 Hz, 1H), 7.10 (ddd, J=2.76 Hz and J=7.08 Hz and J=9.08 Hz, 1H), 5.76 (d, J=17.2 Hz, 1H), 5.55 (d, J=10.8 Hz, 1H).

### Compound 42

### Preparation of 5-[3-Propyl-4-(toluene-4-sulfonylamino)-phenoxy]-2-(toluene-4-sulfonylamino)-benzoic acid

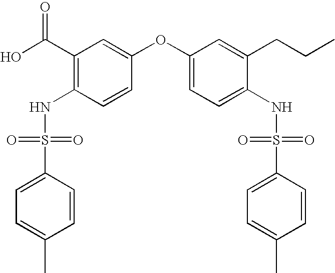

A solution of 5-[3-propyl-4-(toluene-4-sulfonylamino)-phenoxy]-2-(toluene-4-sulfonyl-amino)-benzoic acid methyl ester (332 mg, 0.55 mmol) in aq. 0.5 M LiOH (5 mL) and THF (5 mL) was stirred at 60° C. for 2 hours. The reaction was cooled to RT and evaporated in vacuo to remove THF. The residue was acidified with aq. 2 M HCl (5 mL). The suspension was filtered, washed extensively with water, and dried at 40° C. in vacuo to obtain the desired product (262 mg, 80%, purity (LC)>95%).

MS: [M−H]⁻=593

NMR: ¹H (DMSO-d6): δ 9.45 (s, 1H), 7.65 (d, J=8.32 Hz, 2H), 7.55-7.48 (m, 3H), 7.38-7.31 (m, 5H), 7.21 (dd, J=3.04 Hz and J=9.08 Hz, 1H), 6.82 (d, J=8.84 Hz, 1H), 6.76 (d, J=2.76 Hz, 1H), 6.67 (dd, J=2.76 Hz, 1H), 2.38-2.32 (m, 8H), 1.31-1.20 (m, 2H), 0.73 (t, J=7.08 Hz, 3H).

### Preparation of 5-[3-Propyl-4-(toluene-4-sulfonylamino)-phenoxy]-2-(toluene-4-sulfonylamino)-benzoic acid methyl ester

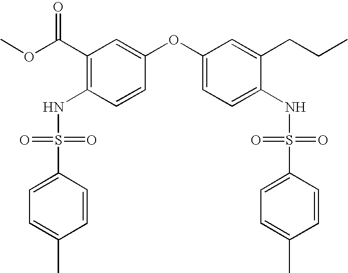

A solution of 5-(4-Amino-3-propyl-phenoxy)-2-(toluene-4-sulfonylamino)-benzoic acid methyl ester (400 mg, 0.88 mmol), tosyl chloride (235 mg, 1.23 mmol), and pyridine (149 mg, 1.88 mmol) in DCM (3 mL) was stirred at 40° C. for 72 hours. The reaction was diluted with CH₂Cl₂ (50 mL) and washed with aq. 0.5 M KHSO₄ (2×50 mL). The aqueous-layers were extracted once more with CH₂CL₂ (50 mL). The combined CH₂CL₂-layers were dried (Na₂SO₄), filtered, and evaporated to dryness in vacuo. The crude product was purified by flash column chromatography (EtOAc/heptane: 1/4 to 1/2) and then triturated with heptane (5 mL) to afford the desired product as a white solid (395 mg, 74%, purity (LC)>95%).

MS: [M−H]⁻=607

NMR: ¹H (CDCl3): δ 10.30 (s, 1H), 7.73-7.66 (m, 3H), 7.60 (d, J=8.32 Hz, 1H), 7.50 (d, J=2.80 Hz, 1H), 7.27-7.21 (m, 4H), 7.19 (d, J=8.56 Hz, 1H), 7.10 (dd, J=3.04 Hz and J=9.08 Hz, 1H), 6.69-6.63 (m, 2H), 3.82 (s, 3H), 2.41 (s, 3H), 2.38 (s, 3H), 2.22 (t, J=7.84 Hz, 2H), 1.40-1.29 (m, 2H), 0.81 (t, J=7.08 Hz, 3H).

### Preparation of 5-(4-Amino-3-propyl-phenoxy)-2-(toluene-4-sulfonylamino)-benzoic acid methyl ester

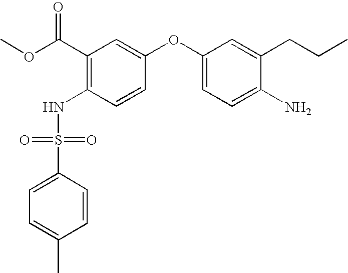

A suspension of 5-[4-nitro-3-((Z)-propenyl)-phenoxy]-2-(toluene-4-sulfonylamino)-benzoic acid methyl ester and 10% Pd—C (10 mg) in THF/MeOH: 1/1 (10 mL) was placed under hydrogen and stirred at RT for 2.5 hours. The reaction was filtered over Kiezelguhr and the residue was rinsed with THF (20 mL). The filtrate was evaporated to dryness in vacuo to the desired amine (382 mg, 100%, purity (LC)>95%).

MS: [M−H]⁻=453.

### Preparation of 5-[4-nitro-3-((Z)-propenyl)-phenoxy]-2-(toluene-4-sulfonylamino)-benzoic acid methyl ester

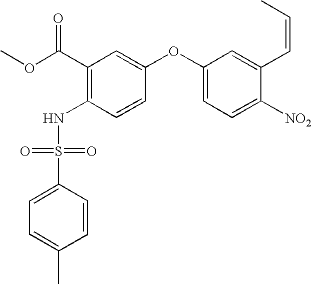

A mixture of methyl 5-hydroxy-2-[(4-methylphenyl)sulfonyl]aminobenzoate (496 mg, 1.54 mmol), 4-fluoro-1-nitro-2-[(Z)-1-propenyl]benzene (279 mg, 1.54 mmol), and potassium carbonate (421 mg, 3.05 mmol) in DMF (10 mL) was stirred at 80° C. for 5 hours. The reaction was diluted with EtOAc (100 mL) and washed with aq. 0.5 M KHSO₄ (100 mL) and brine (2×100 mL). The aqueous-layers were extracted once more with EtOAc (100 mL). The combined EtOAc-extracts were dried (Na₂SO₄), filtered, and evaporated to dryness in vacuo. The crude product was purified by flash column chromatography (EtOAc/heptane: 1/10) to obtain the desired product as a clear oil (427 mg, 57%, purity (LC)>95%).

MS: [M−H]⁻=481.

### Preparation of 4-fluoro-1-nitro-2-[(Z)-1-propenyl]benzene

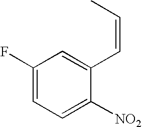

At RT and under N₂, Pd(PPh3)4 (153 mg, 0.13 mmol) was added to a mixture of cis-propenylboronic acid (492 mg, 5.73 mmol), 4-fluoro-2-bromonitrobenzene (843 mg, 3.83 mmol), cesium fluoride (1.20 g, 7.90 mmol) in DME (5 mL, degassed by purging). The reaction mixture was warmed at reflux for 16 hours. The reaction mixture was filtered over Kiezelguhr. The residue was rinsed with EtOAc (50 mL). The filtrate was washed with brine (50 mL). The aqueous-layer was extracted once more with EtOAc (50 mL). The combined EtOAc-extracts were dried (Na₂SO₄), filtered, and evaporated to dryness in vacuo. The crude product was purified by column chromatography to afford 4-fluoro-1-nitro-2-[(Z)-1-propenyl]benzene as a yellow oil (473 mg, 68%, purity (GC)=85%).

NMR: ¹H (CDCl3): δ 8.10 (dd, J=5.04 Hz and J=8.84 Hz, 1H), 7.12-7.04 (m, 2H), 6.72 (dd, J=1.52 Hz and J=11.6 Hz, 1H), 5.99 (dq, J=11.6 Hz, J=7.08 Hz, 1H), 1.76 (dd, J=1.52 Hz and J=7.08 Hz, 3H).

### Compound 43

### Preparation of (5-fluoro-2-nitro-phenyl)-propyl-amine

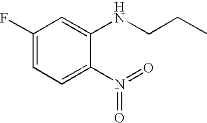

At RT and under nitrogen, Pd(OAc)2 (24 mg, 0.11 mmol) and BINAP (74 mg, 0.12 mmol) were added to a solution of butylamine (173 mg, 2.37 mmol) and 4-fluoro-2-bromonitrobenzene (460 mg, 2.09 mmol) in toluene (3 mL, degassed bu purging). The reaction mixture was heated at 100° C. for 3 minutes and then cooled to 0° C. NaOtBu (271 mg, 2.82 mmol) was added and immediately the reaction mixture turned red. The reaction was stirred at 70° C. for 16 h. The reaction was cooled to RT and filtered over Kiezelguhr. The residue was rinsed with EtOAc (30 mL). The EtOAc-layer was washed with brine (30 mL). The aqueous-layer was extracted once more with EtOAc (30 mL). The combined EtOAc-extracts were dried (Na₂SO₄), filtered, and evaporated to drynessin vacuo. The crude product was purified by flash column chromatography (EtOAc/heptane: 1/100 to 1/30) to afford (5-fluoro-2-nitro-phenyl)-propyl-amine as a yellow oil (333 mg, 75%, purity (GC)=85%).

MS: [M]⁺=212.

### 5-(4-nitro-3-propylamino-phenoxy)-2-(toluene-4-sulfonylamino)-benzoic acid methyl ester

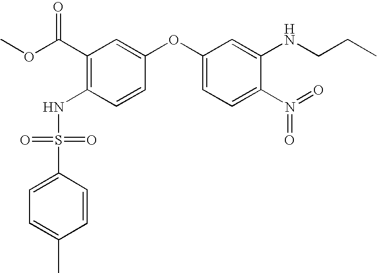

A suspension of methyl 5-hydroxy-2-[(4-methylphenyl)sulfonyl]aminobenzoate (392 mg, 1.22 mmol), (5-fluoro-2-nitro-phenyl)-propyl-amine (242 mg, 1.22 mmol) and anh. potassium carbonate (371 mg, 2.69 mmol) in DMF (15 mL) was stirred at 80° C. After 7 hours, the reaction was diluted with ethyl acetate (150 mL) and washed with aq. 0.5N KHSO₄ (100 mL), aqueous saturated ammonium chloride (100 mL), and brine (100 mL). The organic layer was dried over anhydrous sodium sulfate and evaporated in vacuo. The oily residue was purified by column chromatography (SiO₂) on elution with heptane/EtOAc from 9:1 to 4:1 to obtain the desired product (481 mg, 79%, purity (LC)>90%).

MS: [M−H]⁻=498

### Preparation of 5-(4-amino-3-propylamino-phenoxy)-2-(toluene-4-sulfonylamino)-benzoic acid methyl ester

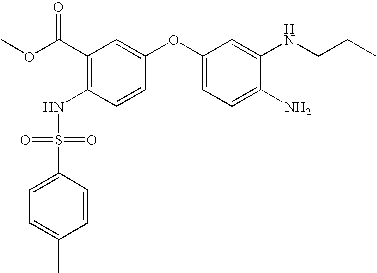

A suspension of 5-(4-nitro-3-propylamino-phenoxy)-2-(toluene-4-sulfonylamino)-benzoic acid methyl ester (481 mg, 0.96 mmol) and 10% palladium on activated carbon (51 mg, 0.048 mmol) in tetrahydrofuran (5mL) and methanol (5 mL) was stirred at room temperature under 1 atmosphere of hydrogen. After 5 hours the reaction was filtered over Kieselguhr and evaporated under reduced pressure to afford the desired product (435 mg, 96%, purity (LC) 84.4%)

MS: [M−H]⁻=468

### Preparation of 5-[3-propylamino-4-(toluene-4-sulfonylamino)-phenoxy]-2-(toluene-4-sulfonylamino)-benzoic acid methyl ester

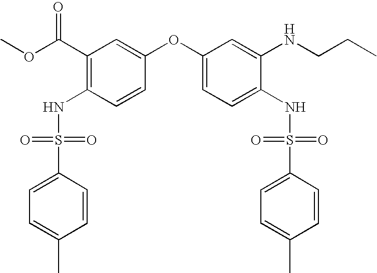

A solution of 5-(4-amino-3-propylamino-phenoxy)-2-(toluene-4-sulfonylamino)-benzoic acid methyl ester (435 mg, 0.93 mmol), p-toluenesulfonylchloride (160 mg, 0.84 mmol) and pyridine (146 mg, 1.85 mmol) in dichloromethane (10 mL) was stirred for 20 hours at 40° C. The reaction was diluted with dichloromethane (50 mL) and washed with aqueous 0.5N potassium hydrogen sulfate (30 mL), saturated sodium hydrogen carbonate (30 mL) and dried over anhydrous sodium sulfate. After concentration under reduced pressure the residue was purified by column chromatography (SiO₂) on gradient elution with heptane/EtOAc from 1:0 to 4:1 to obtain the desired product. The solid was dried in vacuo for 16 hours at 40° C. (310 mg, 53%, purity (LC)>95%).

MS: [M−H]⁻=622

NMR: ¹H (CDCl₃): δ 10.30 (bs, 1H), 7.70 (d, J=8.32 Hz, 2H), 7.66 (d, J=9.32 Hz, 1H), 7.64 (d, J=9.12 Hz, 2H), 7.51 (d, J=2.76 Hz, 1H) 7.27 (d, J=8.84 Hz, 2H), 7.22 (d, J=8.08 Hz, 2H), 7.12 (dd, J=2.76 Hz and 8.84 Hz, 1H), 6.32 (d, J=8.32 Hz, 1H), 6.19 (d, J=2.76, 1H), 5.87 (dd, J=2.56 Hz and 8.60 Hz, 1H), 5.75 (bs, 1H), 4.68 (t, J=5.30 Hz, 1H), 3.82 (s, 3H), 2.95-2.90 (m, 2H), 2.43 (s, 3H), 2.38 (s, 3H), 1.61-1.52 (m, 2H), 0.95 (t, J=7.32 Hz, 3H)

### Preparation of 5-[3-Propylamino-4-(toluene-4-sulfonylamino)-phenoxy]-2-(toluene-4-sulfonylamino)-benzoic acid

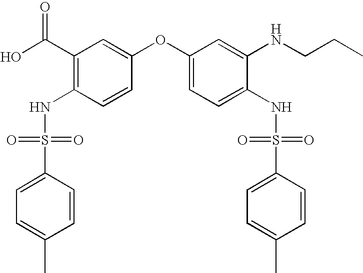

A mixture of 5-[3-propylamino-4-(toluene-4-sulfonylamino)-phenoxy]-2-(toluene-4-sulfonylamino)-benzoic acid methyl ester (240 mg, 0.39 mmol), LiOH (65 mg, 1.55 mmol), water (2 mL) and THF (2 mL) was stirred for 4 hours at 60° C. The reaction was acidified to pH=1 with aq. 1N HCl and the THF was evaporated under reduced pressure. The suspension was extracted with DCM (2×50 mL) and the extracts were dried over anhydrous sodium sulfate. After concentration of the organic solution, the residue was purified by flash-column chromatography (SiO₂) on gradient elution with dichloromethane/methanol from 100:0 to 98:2 to obtain the desired product. The solid was dried in vacuo for 16 hours at 40° C. (193 mg, 81%, purity (LC)>95%).

MS: [M−H]⁻=608

NMR: ¹H (DMSO-d6): δ 10.83 (bs, 1H), 9.18 (bs, 1H), 7.65 (d, J=8.32 Hz, 2H), 7.51 (d, J=8.08 Hz, 3H) 7.35 (d, J=8.84 Hz, 3H), 7.31 (d, J=2.76 Hz, 2H), 7.23 (dd, J=3.04 Hz and 8.84 Hz, 1H), 6.62 (d, J=8.60 Hz, 1H), 6.09 (d, J=2.56 Hz, 1H), 5.96 (dd, J=2.52 Hz and 8.56 Hz, 1H), 4.94 (bs, 1H), 2.73 (t, J=6.70 Hz, 2H), 2.35 (s, 3H), 2.34 (s, 3H), 1.38-1.29 (m, 2H), 1.09 (t, J=6.94 Hz, 3H)

### Compound 44

### Preparation of 5-(3-carboxy-4-[(4-methylphenyl)sulfonyl]aminophenoxy)-2-[(4-methylphenyl)sulfonyl]aminobenzoic acid

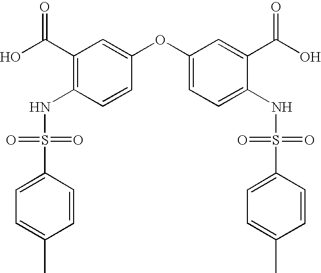

A solution of methyl 5-(3-(methoxycarbonyl)-4-[(4-methylphenyl)sulfonyl]amino-phenoxy)-2-[(4-methylphenyl)sulfonyl]aminobenzoate (164 mg, 0.262 mmol) in aq. 0.25 M LiOH (4 mL) and THF (2 mL) was stirred at RT for 20 hours. An additional amount of aq. 0.25 M LiOH (2 mL) was added and the reaction was continued at 60° C. for 5 hours. The reaction mixture was cooled to RT and aq. 1 N HCL (4 mL) was added. The reaction mixture was evaporated to dryness in vacuo. The residue was dissolved in aq. 1 N HCl (20 mL) and DCM (20 mL). The DCM-layer was isolated and the water layer was extracted once more with DCM (20 mL). The combined DCM-layers were dried (Na₂SO₄), filtered, and evaporated to dryness to afford the desired product (151 mg, 97%, purity (LC)>95%).

MS: [M−H]⁻=595.

NMR: ¹H (DMSO-d6): δ 10.79 (s, 1H), 7.64 (d, J=8.33 Hz, 4H), 7.52 (d, J=9.09 Hz, 2H), 7.38-7.33 (m, 6H), 7.26 (dd, J=3.03 Hz and J=9.09 Hz, 2H), 2.34 (s, 6H).

### Preparation of methyl 5-(3-(methoxycarbonyl)-4-[(4-methylphenyl)sulfonyl]amino-phenoxy)-2-[(4-methylphenyl)sulfonyl]aminobenzoate

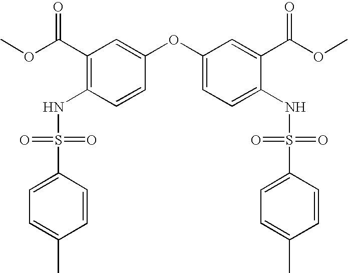

A solution of methyl 5-[4-amino-3-(methoxycarbonyl)phenoxy]-2-[4-methylphenyl)-sulfonyl]aminobenzoate (580 mg, 1.23 mol), tosyl chloride (294 mg, 1.54 mmol), and pyridine (196 mg, 2.48 mmol) in DCM (5 mL) was warmed at reflux for 18 hours. The reaction mixture was diluted with DCM (75 mL) and washed with aq. 0.5 N KHSO₄ (50 mL) and aq sat. NaHCO₃ (50 mL). The aqueous-layers were extracted once more with DCM (50 mL). The combined DCM-layers were dried (Na₂SO₄), filtered, and evaporated to dryness in vacuo. The crude product was by flash column chromatography (EtOAc/heptane: 1/5 to 1/2) to nearly pure product. This product was crystallized from EtOAc/heptane to obtain the desired product as small white crystals (375 mg, 49%, purity (LC)>95%).

MS: [M−H]⁻=623.

NMR: ¹H (CDCl3): δ 10.31 (s, 1H), 7.71 (d, J=8.08 Hz, 4H), 7.67 (d, J=9.08 Hz, 2H), 7.44 (d, J=3.04 Hz, 2H), 7.24 (d, J=8.08 Hz, 4H), 7.06 (dd, J=3.04 Hz and J=9.08 Hz, 2H), 3.82 (s, 6H), 2.39 (s, 6H).

### Preparation of methyl 5-[4-amino-3-(methoxycarbonyl)phenoxy]-2-[(4-methylphenyl)-sulfonyl]aminobenzoate

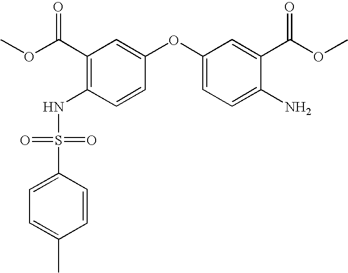

At RT and under nitrogen, a solution of methyl 5-[4-nitro-3-(methoxycarbonyl)-phenoxy]-2-[(4-methylphenyl)sulfonyl]aminobenzoate (995 mg, 1.99 mmol) in THF/MeOH: 1/1 (20 mL) was added gradually to a suspension of iron (444 mg, 7.95 mmol) and ammonium chloride (435 mg, 8.13 mmol) in water (10 mL). The reaction was heated at 70° C. for 7 hours. The reaction was cooled to RT and then the reaction mixture was filtered over Kiezelguhr. The residue was rinsed with EtOAc (50 mL). The filtrate was washed with aq. sat. NaHCO₃ (50 mL) and brine (50 mL). The organic layer was dried (Na₂SO₄), filtered, and evaporated to dryness in vacuo. The crude product was purified by flash column chromatography (EtOAc/heptane: 1/4 to 1/1) to obtain the desired product (580 mg, 62%, purity (LC)>95%).

MS: [M+H]⁺=471

### Preparation of methyl 5-[4-nitro-3-(methoxycarbonyl)phenoxy]-2-[(4-methylphenyl)-sulfonyl]aminobenzoate

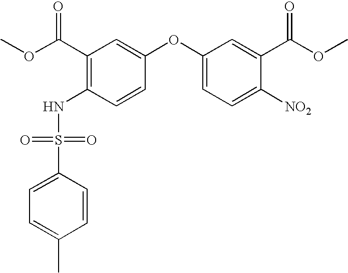

A mixture of methyl 5-hydroxy-2-[(4-methylphenyl)sulfonyl]aminobenzoate (643 mg, 2.00 mmol), methyl 4-fluoro-2-nitrobenzoate (400 mg, 2.01 mmol), and potassium carbonate (561 mg, 4.06 mmol) in DMF (10 mL) was stirred at 80° C. for 3 hours. The reaction mixture was diluted with EtOAc (50 mL) and washed with brine (3×100 mL). The aqueous-layers were extracted with EtOAc (1×50 mL). The EtOAc-extracts were dried (Na₂SO₄), filtered, and evaporated to dryness in vacuo to obtain the desired product as a yellow foam (1.06 g, 106%, purity (LC)>95%).

MS: [M−H]⁻=499.

### Preparation of methyl 4-fluoro-2-nitrobenzoate

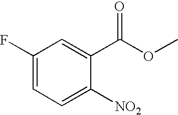

At RT, SOCl₂ (400 mg, 3.36 mmol) was added gradually to an ice-cold solution of solution of commercially available 5-fluoro-2-nitrobenzoic acid (558 mg, 3.01 mmol) in MeOH (10 mL). The reaction was heated at reflux for 24 hours. The reaction was cooled to 0° C., SOCl₂ (440 mg) was added and the reaction was continued at reflux for 24 hours. The reaction was cooled to RT and evaporated to dryness in vacuo. The residue was dissolved in EtOAc (40 mL) and washed with aq. sat. NaHCO₃ (1×40 mL) and brine (1×40 mL). The aqueous layers were extracted with EtOAc (1×40 mL). The combined EtOAc-extracts were dried (Na₂SO₄), filtered, and evaporated to dryness in vacuo to obtain methyl 4-fluoro-2-nitrobenzoate as a yellow oil (433 mg, 72%, purity (LC) n.d.).

NMR: ¹H (CDCl3): δ 8.03 (dd, J=4.56 Hz and J=8.84 Hz, 1H), 7.39 (dd, J=2.76 Hz and J=7.84 Hz, 1H), 7.31 (ddd, J=2.76 Hz and J=7.32 Hz and J=9.12 Hz, 1H), 3.95 (s, 3H).

### Compound 45

### Preparation of 5-(4-{[(Pyridine-4-carbonyl)-amino]-methyl}-phenoxy)-2-(toluene-4-sulfonylamino)-benzoic acid

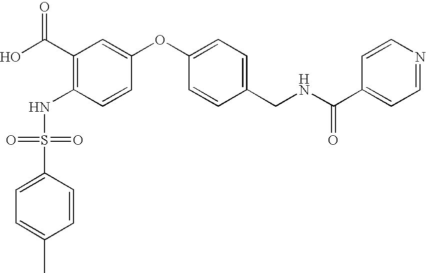

5-(4-{[(Pyridine-4-carbonyl)-amino]-methyl}-phenoxy)-2-(toluene-4-sulfonylamino)-benzoic acid methyl ester (63 mg, 0.119 mmol) was dissolved in a mixture of 1:1 water/THF (10 ml). Added LiOH (20 mg, 0.474 mmol). Stirred at room temperature for 18 hours. There was still some start product present. Stirred at 50° C. for 5 hours. Cooled, acidified with 0.5 M KHSO₄. Added aq. sat. NaCl and extracted with CH₂Cl₂. Washed the CH₂Cl₂ with aq. sat. NaCl. Dried over Na₂SO₄, filtered off and concentrated in vacuo. Gave 63 mg (>100%) product.

LC: >95%

MS: [M−H]⁺=516

NMR: ¹H (300 MHz, dmso-d6)

δ 10.77 (s, 1H), 9.28 (t, J=7.60 Hz, 1H), 8.71 (s, 2H), 7.77 (d, J=6.40 Hz, 2H), 7.62 (d, J=10.8 Hz, 2H), 7.49 (d, J=11.6 Hz, 1H), 7.34-7.30 (m, 5H), 7.23 (dd, J=4 Hz and J=12 Hz, 1H), 6.93 (d, J=8.00 Hz, 2H), 4.46 (d, J=8.04 Hz, 2H), 2.33 (s, 3H)

### Preparation of 5-(4-{[(Pyridine-4-carbonyl)-amino]-methyl}-phenoxy)-2-(toluene-4-sulfonylamino)-benzoic acid methyl ester

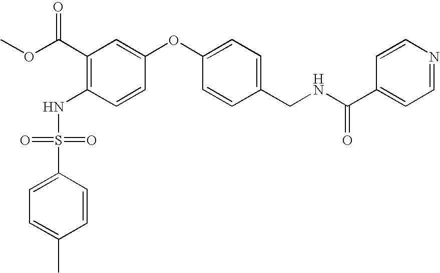

Dissolved 5-(4-Aminomethyl-phenoxy)-2-(toluene-4-sulfonylamino)-benzoic acid methyl ester (80 mg, 0.188 mmol) and commercially available isonicotinic acid (28 mg, 0.226) in CH₂Cl₂ (5 ml). Added EDCI (54 mg, 0.282 mmol) and HOAt (26 mg, 0.188 mmol). Stirred under Nitrogen at room temperature for 18 hours. Added CH₂Cl₂ washed with 0.5 N NaOH (2×25 ml). Subsequently washed with 5% aq. citric acid (2×25 ml) and aq. sat. NaCl (25 ml). Dried over Na₂SO₄, filtered off and concentrated in vacuo. Gave 63 mg (63%) product.

LC: >95%

MS: [M+H]=532

### Preparation of 5-(4-Aminomethyl-phenoxy)-2-(toluene-4-sulfonylamino)-benzoic acid methyl ester

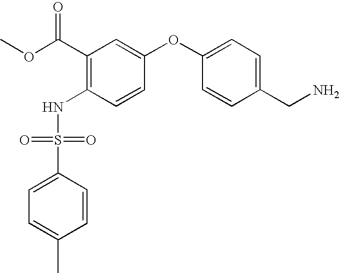

Dissolved 5-(4-Cyanophenoxy)-2-(toluene-4-sulfonylamino)-benzoic acid methyl ester (3.1 g, 7.34 mmol) in MeOH (100 ml) and placed in an autoclave. Added Et₃N (2.1 ml, 14.6 mmol) and Raney Nickel (catalytic amount). Placed under Hydrogen (7 bar). Stirred at room temperature for 18 h. Flushed the solution with Nitrogen. Filtered off over kieselguhr and washed with MeOH. The filtrate was concentrated in vacuo. Purified by a silica gel column, eluens CH₂Cl₂:MeOH 95:5 (+0.2% Et₃N). Most of the impurities were eluted off Changed to eluens CH₂Cl₂:MeOH 9:1 (+0.2% Et3N). Concentrated the product in vacuo and stripped with CH₂Cl₂. Gave 1.56 g (50%) off white/yellow foam.

LC: >95%

MS: [M+1]⁺=427

NMR: ¹H (300 MHz, dmso-d6)

δ7.57 (d, J=11.2 Hz), 7.35-7.26 (m, 5H), 7.14-7.07 (m, 2H), 6.91-6.88 (m, 2H), 3.77 (s, 2H), 3.69 (s, 3H), 2.32 (s, 3H)

### Preparation of 5-(4-Cyano-phenoxy)-2-(toluene-4-sulfonylamino)-benzoic acid methyl ester

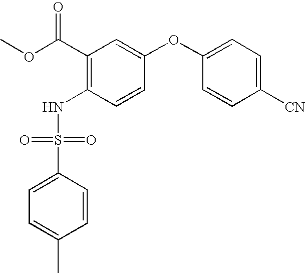

5-Hydroxy-2-(toluene-4-sulfonylamino)-benzoic acid methyl ester (7.73 g, 24.0 mmol) was dissolved in dry dmso (100 ml). Added K₂CO₃ (8.29 g, 60 0 mmol) and commercially available 4-fluorobenzonitril (3.05 g, 25.2 mmol). Heated to 80° C. for 18 hours. Distilled the dmso off. Added water (200 ml), extracted with EtOAc (200 and 2×100 ml). Washed EtOAc with water (2×100 ml) and aq. sat. NaCl (100 ml). Dried over Na₂SO₄, filtered off and concentrated in vacuo. Purified by a silica gel column, coated the crude product on isolute, eluens Heptane:EtOAc 7:3. Stripped the isolated product with CH₂Cl₂. Gave 3.1 g (31%) off white foam.

LC: >95%

MS: [M+H]⁺=423

NMR: ¹H (300 MHz, dmso-d6)

δ 10.14 (s, 1H), 7.84-7.79 (m, 2H), 7.64-7.60 (m, 2H), 7.48-7.45 (m, 2H), 7.39-7.33 (m, 3H), 7.08-7.03 (m, 2H), 3.74 (s, 3H), 2.34 (s, 3H)

### Compound 46

### Preparation of 2-(Toluene-4-sulfonylamino)-5-{4-[(toluene-4-sulfonylamino)-methyl]-phenoxy}-benzoic acid

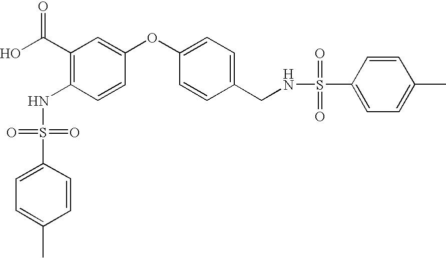

2-(Toluene-4-sulfonylamino)-5-{4-[(toluene-4-sulfonylamino)-methyl]-phenoxy}-benzoic acid methyl ester (42 mg, 0.072 mmol) was dissolved in water/THF (1:1 v/v, 10 ml). Added LiOH (15 mg, 0.361 mmol). Stirred at 40° C. for 18 hours. Distilled most of the THF off. Acidified with 2N HCl, filtered off and washed with water (25 ml). Dried in vacuo at 40° C. for 6 hours. Gave 28 mg (68%) product.

LC: >95%

MS: [M−H]⁻=565

NMR: 1H (DMSO-d6)

δ 8.07 (t, J=6.56 Hz, 1H), 7.66 (m, 4H), 7.51 (d, J=9.12 Hz, 1H), 7.37-7.33 (m, 5H), 7.23-7.19 (m,1H), 6.88 (d, J=8.60 Hz, 2H), 3.92 (d, J=6.08 Hz, 2H), 2.35 (d, J=6.04 Hz, 6H)

### Preparation of 2-(Toluene-4-sulfonylamino)-5-{4-[(toluene-4-sulfonylamino)-methyl]-phenoxy}-benzoic acid methyl ester

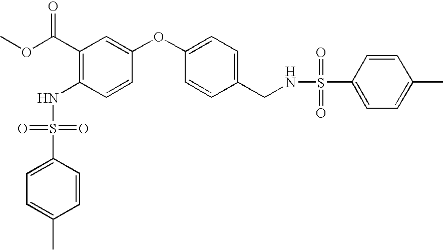

5-(4-Aminomethyl-phenoxy)-2-(toluene-4-sulfonylamino)-benzoic acid methyl ester (80 mg, 0.188 mmol) was dissolved in CH₂Cl₂ (5 ml). Added pyridine (31 μl, 0.376 mmol) and sulfonyl chloride (37 mg, 0.196 mmol). Stirred at room temperature for 48 hours. Added CH₂Cl₂, washed with 0.5 N HCl (2×25 ml) added some NaCl and 0.5 N NaOH (1×25 ml) and brine (1×25 ml). Dried over Na₂SO₄ and concentrated under reduced pressure. Preparative plate (1 mm layer thickness), eluens Heptane:EtOAc 1:1. Removed product from the silica gel using CH₂Cl₂:MeOH 9:1, filtered off and concentrated in vacuo. Gave 42 mg (39%) product.

LC: >95%

MS: [M−H]⁻=579

### Compound 47

### Preparation of 5-{4-[(2-Phenoxy-acetylamino)-methyl]-phenoxy}-2-(toluene-4-sulfonylamino)-benzoic acid

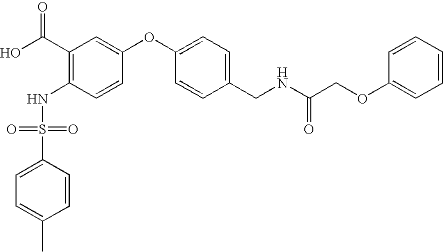

5-{4-[(2-Phenoxy-acetylamino)-methyl]-phenoxy}-2-(toluene-4-sulfonylamino)-benzoic acid methyl ester (74 mg, 0.132 mmol) was dissolved in water/THF (1:1 v/v, 10 ml). Added LiOH (22 mg, 0.529 mmol). Stirred at 40° C. for 18 hours. Distilled most of the THF off. Cooled, acidified with 2N HCl. Added CH₂Cl₂ and washed with brine (25 ml). Dried over Na₂SO₄ and concentrated under vacuo. Gave 74 mg (>100%) product.

LC: >95%

MS: [M−H]⁻=545

NMR: 1H (DMSO-d6)

δ 8.66 (t, J=6.08 Hz, 1H), 7.66 (d, J=8.56 Hz, 2H), 7.52 (d, J=8.60 Hz, 1H), 7.37-7.25 (m, 9H), 6.97-6.92 (m,5H), 4.53 (s, 2H), 4.32 (d, J=6.04 Hz, 2H), 2.34 (s, 3H)

### Preparation of 5-{4-[(2-Phenoxy-acetylamino)-methyl]-phenoxy}-2-(toluene-4-sulfonylamino)-benzoic acid methyl ester

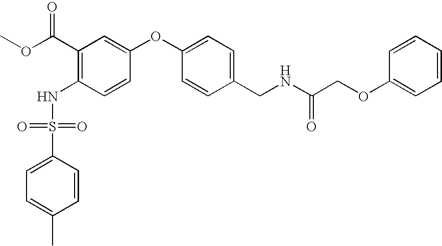

5-(4-Aminomethyl-phenoxy)-2-(toluene-4-sulfonylamino)-benzoic acid methyl ester (80 mg, 0.188 mmol) was dissolved in CH₂Cl₂ (5 ml). Added Pyridine (31 μl 0.376 mmol) and sulfonyl chloride (33 mg, 0.196 mmol). Stirred at room temperature for 18 hours. Added CH₂Cl₂, washed with 0.5 N HCl (25 ml), 0.5 N NaOH (25 ml) and brine (25 ml). Dried over Na₂SO₄ and concentrated under vacuo. Preparative plate (2 mm layer thickness), eluens Heptane:EtOAc 1:1. Removed product from the silica gel using CH₂Cl₂:MeOH 9:1, filtered off and concentrated in vacuo. Gave 74 mg (70%).

LC: >95%

MS: [M−H]⁻=559

### Compound 48

### Preparation of 5-{4-[(4-Acetyl-benzenesulfonylamino)-methyl]-phenoxy}-2-(toluene-4-sulfonylamino)-benzoic acid

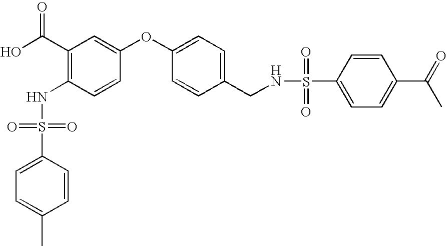

5-{4-[(4-Acetyl-benzenesulfonylamino)-methyl]-phenoxy}-2-(toluene-4-sulfonyl-amino)-benzoic acid methyl ester (49 mg, 0.0806 mmol) was dissolved in water/THF (1:1 v/v, 10 ml). Added LiOH (22 mg, 0.322 mmol). Stirred at 40° C. for 18 hours. Distilled most of the THF off. Cooled, acidified with 2N HCl. Added CH₂Cl₂ and washed with brine (25 ml). Dried over Na₂SO₄ and concentrated under vacuo. Gave 43 mg (90%).

LC: >95%

MS: [M−H]⁻=593

NMR: 1H (DMSO-d6)

δ 8.37 (t, J=6.56 Hz, 1H), 8.10 (d, J=8.60 Hz, 2H), 7.89 (d, J=8.60 Hz, 2H), 7.66 (d, 8.08 Hz, 2H), 7.52 (d, J=9.12 Hz, 1H), 7.37-7.33 (m, 3H), 7.23-7.19 (m,3H), 6.88 (d, J=8.56 Hz, 2H), 4.00 (d, J=6.56 Hz, 2H), 2.62 (s, 3H), 2.35 (s, 3H)

### Preparation of 5-{4-[(4-Acetyl-benzenesulfonylamino)-methyl]-phenoxy}-2-(toluene-4-sulfonylamino)-benzoic acid methyl ester

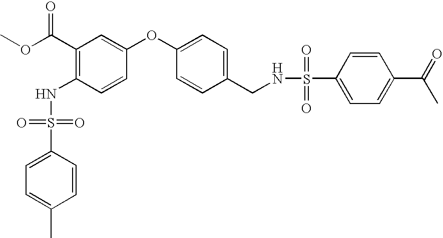

5-(4-Aminomethyl-phenoxy)-2-(toluene-4-sulfonylamino)-benzoic acid methyl ester (80 mg, 0.188 mmol) was dissolved in CH₂Cl₂ (5 ml). Added Pyridine (31 μl, 0.376 mmol) and sulfonyl chloride (43 mg, 0.196 mmol). Stirred at room temperature for 18 hours. Added CH₂Cl₂, washed with 0.5 N HCl (2×25 ml) added some NaCl and 0.5 N NaOH (1×25 ml) and brine (1×25 ml). Dried over Na₂SO₄ and concentrated under vacuo. Preparative plate (1 mm layer thickness), eluens Heptane:EtOAc 1:1. Removed product from the silica gel using CH₂Cl₂:MeOH 9:1, filtered off and concentrated in vacuo. Gave 28 mg (24%).

LC: >95%

MS: [M−H]⁻=607

### Compound 49

### Preparation of 5-{4-[(4-Nitro-benzenesulfonylamino)-methyl]-phenoxy}-2-(toluene-4-sulfonylamino)-benzoic acid

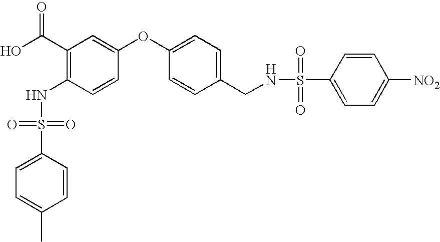

5-{4-[(4-Nitro-benzenesulfonylamino)-methyl]-phenoxy}-2-(toluene-4-sulfonyl-amino)benzoic acid methyl ester (31 mg, 0.051 mmol) was dissolved in water/THF (1:1 v/v, 10 ml). Added LiOH (9 mg, 0.202 mmol). Stirred at 40° C. for 18 hours. Distilled most of the THF off Cooled, acidified with 2N HCl. Added CH₂Cl₂ and washed with brine (25 ml). Dried over Na₂SO₄ and concentrated under vacuo. Gave 29 mg (95%).

LC: >95%

MS: [M−H]⁻=596

NMR: 1H (DMSO-d6)

δ 8.56 (t, J=5.84 Hz, 1H), 8.37 (d, J=8.84 Hz, 2H), 7.99 (d, J=8.84 Hz, 2H), 7.65 (d, 8.08 Hz, 2H), 7.48 (d, J=8.84 Hz, 1H), 7.34(d, J=7.80 Hz, 3H), 7.19 (d, J=8.60 Hz, 2H), 7.12 (d, J=9.08 Hz, 1H), 6.85 (d, J=8.56 Hz, 2H), 4.03 (d, J=6.32 Hz, 2H), 2.34 (s, 3H)

### Preparation of 5-{4-[(4-Nitro-benzenesulfonylamino)-methyl]-phenoxy}-2-(toluene-4-sulfonylamino)-benzoic acid methyl ester

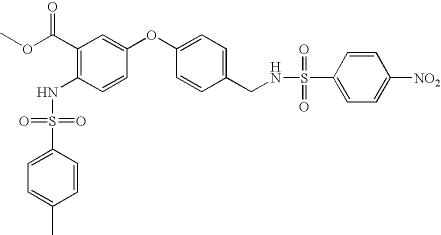

5-(4-Aminomethyl-phenoxy)-2-(toluene-4-sulfonylamino)-benzoic acid methyl ester (80 mg, 0.188 mmol) was dissolved in CH₂Cl₂ (5 ml). Added Pyridine (31 μl, 0.376 mmol) and sulfonyl chloride (43 mg, 0.196 mmol). Stirred at room temperature for 18 hours. Added CH₂Cl₂, washed with 0.5 N HCl (2×25 ml) added some NaCl and 0.5 N NaOH (1×25 ml) and brine (1×25 ml). Dried over Na₂SO₄ and concentrated under vacuo. Preparative plate (1 mm layer thickness), eluens Heptane:EtOAc 1:1. Removed product from the silica gel using CH₂Cl₂:MeOH 9:1, filtered off and concentrated in vacuo. Gave 49 mg (43%).

LC: >95%

MS: [M−H]⁻=610

1. A homogeneous time resolved fluorescence-based test system comprising a first helical labeled polypeptide consisting essentially of a sequence derived from the heptad-repeat 1 (HR1) region of Human Immunodeficiency Virus (HIV); a second helical labeled polypeptide consisting essentially of a sequence derived from the heptad-repeat 2 (HR2) region of HIV.

2. A homogeneous time resolved fluorescence-based test system according to claim 1 wherein the first helical polypeptide is labeled with a light emitting fluorophore and the second helical polypeptide is labeled with an ultra-violet excitable fluorophore.

3. A homogeneous time resolved fluorescence-based test system according to claim 1 wherein the first helical polypeptide is labeled with an ultra-violet excitable fluorophore and the second helical polypeptide is labeled with a light emitting fluorophore.

4. A homogeneous time resolved fluorescence-based test system according to any of the claims 1-3 wherein the first helical polypeptide consists essentially of the sequence of IQN36 (SEQ ID NO: 1); the second helical polypeptide consists essentially of the sequence of C34 (SEQ ID NO: 2).

5. A homogeneous time resolved fluorescence-based test system according to claim 4 wherein said IQN36 is labeled with a light emitting fluorophore and said C34 is labeled with an ultra-violet excitable fluorophore.

6. A homogeneous time resolved fluorescence-based test system according to claim 4 wherein said C34 is labeled with a light emitting fluorophore and said IQN36 is labeled with an ultra-violet excitable fluorophore.

7. A homogeneous time resolved fluorescence-based test system according to claim 5 wherein said IQN36 sequence comprises a linker between the label and the IQ-moiety of the IQN36 sequence.

8. A homogeneous time resolved fluorescence-based test system according to claim 7 wherein the linker is attached to the N-terminal IQ-end of the IQN36 sequence.

9. A homogeneous time resolved fluorescence-based test system according to claim 7 or 8 wherein said linker is selected from the group of an antibody-antibody complex, antibody-antigen complex or streptavidin-biotin system.

10. A homogeneous time resolved fluorescence-based test system according to any of the claims 2-9 wherein the ultra-violet excitable fluorophore is selected from the group of lanthanides and wherein the light emitting fluorophore matches the excitation wavelength of the selected lanthanide.

11. A homogeneous time resolved fluorescence-based test system according to claim 10 wherein the light emitting fluorophore is allophycocyanin, preferably streptavidin-allophycocyanin, and the ultra-violet excitable fluorophore is europium.

12. A homogeneous time resolved fluorescence-based test system according to claim 10 wherein the light emitting fluorophore is selected from the group of alexa fluor 546, rhodamine or Cy3 and wherein the ultra-violet excitable fluorophore is terbium.

13. Method for identifying a compound that interferes with the formation of the HIV six-helix bundle of gp41 comprising:
providing a first helical polypeptide consisting essentially of the sequence of IQN36 (SEQ ID NO:1);
providing a second helical polypeptide consisting essentially of the sequence of C34 (SEQ ID NO: 2) wherein said IQN36 is labeled with a light emitting fluorophore and said C34 is labeled with an ultra-violet excitable fluorophore;
providing a test composition comprising the compound;
measuring the degree of complex formation between the first helical polypeptide and the second helical polypeptide in the presence of the test composition comprising the compound using fluorescence resonance energy transfer; and
comparing the measured degree of complex formation to the degree of complex formation between the first helical polypeptide and the second helical polypeptide in the absence of the test composition comprising the compound to identify the compound that interferes with the formation of the HIV six-helix bundle of gp41.

14. Method for identifying a compound that interferes with the formation of the HIV six-helix bundle of gp41 according to claim 13 wherein said IQN36 sequence comprises a linker between the label and the IQ-moiety of the IQN36 sequence.

15. Method for identifying a compound that interferes with the formation of the HIV six-helix bundle of gp41 according to claim 14 wherein the linker is attached to the N-terminal IQ-end of the IQN36 sequence.

16. Method for identifying a compound that interferes with the formation of the HIV six-helix bundle of gp41 according to claim 14 or 15 wherein said linker is selected from the group of an antibody-antibody complex, antibody-antigen complex or streptavidin-biotin system.

17. Method for identifying a compound that interferes with the formation of the HIV six-helix bundle of gp41 according to any of the claims 13-16 wherein the ultra-violet excitable fluorophore is selected from the group of lanthanides and wherein the light emitting fluorophore matches the excitation wavelength of the selected lanthanide.

18. Method for identifying a compound that interferes with the formation of the HIV six-helix bundle of gp41 according to claim 17 wherein the light emitting fluorophore is allophycocyanin, preferably streptavidin-allophycocyanin, and the ultra-violet excitable fluorophore is europium.

19. Method for identifying a compound that interferes with the formation of the HIV six-helix bundle of gp41 according to claim 17 wherein the light emitting fluorophore is selected from the group of alexa fluor 546, rhodamine or Cy3 and wherein the ultra-violet excitable fluorophore is terbium.

20. Method for identifying the mechanism of inhibition of the formation of the HIV six-helix bundle of gp41 comprising:
providing a first helical polypeptide consisting essentially of the sequence of IQN36 (SEQ ID NO:1);
providing a second helical polypeptide consisting essentially of the sequence of C34 (SEQ ID NO: 2) wherein said IQN36 is labeled with a light emitting fluorophore and said C34 is labeled with an ultra-violet excitable fluorophore;
providing a test composition comprising a compound that interferes with the formation of the six-helix bundle of gp41;
measuring the degree of complex formation between the first helical polypeptide and the second helical polypeptide in the presence of the test composition comprising a compound that interferes with the formation of the six-helix bundle of gp41 using fluorescence resonance energy transfer; and
comparing the measured degree of complex formation to the degree of complex formation between the first helical polypeptide and the second helical polypeptide in the absence of the test composition comprising the compound that interferes with the formation of the six-helix bundle of gp41 to identify the mechanism of inhibition of the formation of the HIV six-helix bundle of gp41.

21. The compounds identified by the method of any of the claims 13-19.

22. Use of the compounds obtained by the method of any of the claims 13-19 for inhibiting the formation of the HIV six-helix bundle of gp41.


SEQUENCE LISTING (5 sequences)

SEQ ID NO: 1 - protein, 66 aa, Human immunodeficiency virus
RMKQIEDKIEEIESKQKKIENEIARIKKLISGIVQQQNNLLRAIEAQQHLLQLTVWGIKQLQARIL

SEQ ID NO: 2 - protein, 34 aa, Human immunodeficiency virus
WMEWDREINNYTSLIHSLIEESQNQQEKNEQELL

SEQ ID NO: 3 - protein, 45 aa, Human immunodeficiency virus
RMKQIEDKIEEIESKQKKIENEIARIKKLLQLTVWGIKQLQARIL

SEQ ID NO: 4 - protein, 45 aa, respiratory syncytial virus
NFYDPLVFPSDEFDASISQVNEKINQSLAFIRKSDELLHNVNAGK

SEQ ID NO: 5 - protein, 36 aa, Human immunodeficiency virus
YTSLIHSLIEESQNQQEKNEQELLELDKWASLWNWF